(19) United States
(12) Patent Application Publication
(10) Pub. No.: US 2026/0092048 A1
(43) Pub. Date: Apr. 2, 2026

(54) SOLID FORMS AND SALTS OF A QUINOLINE CGAS ANTAGONIST COMPOUND

(71) Applicants: ImmuneSensor Therapeutics, Inc., Dallas, TX (US); THE BOARD OF REGENTS OF THE UNIVERSITY OF TEXAS SYSTEM, Austin, TX (US)

(72) Inventors: Jian Qiu, Dallas, TX (US); Qi Wei, Dallas, TX (US); Heping Shi, Dallas, TX (US); Lijun Sun, Dallas, TX (US); Zhijian Chen, Dallas, TX (US)

(21) Appl. No.: 19/316,546

(22) Filed: Sep. 2, 2025

Related U.S. Application Data
(60) Provisional application No. 63/689,562, filed on Aug. 30, 2024.

(51) Int. Cl. C07D 401/14

(52) U.S. Cl. CPC C07D 401/14

(57) ABSTRACT

The present disclosure provides compounds that are cGAS antagonists, solid forms of the compounds, methods of preparation of the compounds and their solid forms, pharmaceutical compositions comprising the compounds and their solid forms, and their use in medical therapy.

### 1. BACKGROUND

Cyclic GMP-AMP synthase (cGAS) (UniProtKB-Q8N884) is an enzyme that acts as a DNA sensor to elicit an immune response to pathogens via activation of the stimulator of interferon genes (STING) receptor. Aberrant activation of cGAS by self-DNA is shown to underlie debilitating and sometimes fatal autoimmune diseases. Knockout studies in animal models have indicated that inhibiting cGAS is a promising approach for therapeutic intervention. Additionally, recent studies have shown that the cGAS-STING pathway plays a key role in the innate immune response to tumors, and stimulation of the pathway is a promising strategy being tested clinically for cancer immunotherapy.

Recently, International Publication No. WO 2023/168367 disclosed (S)-3-((1-(7-bromo-6-chloro-4-(1H-1,2,4-triazol-1-yl)quinolin-2-yl)pyrrolidin-2-yl)methoxy)propanoic acid (“Compound A”):

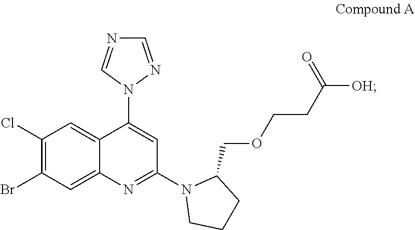 [SMILES: O=C(O)CCOC[C@@H]1CCCN1c1cc(-n2cncn2)c2cc(Cl)c(Br)cc2n1]

which exhibited superior cGAS antagonist activity. Compound A, amongst other cGAS inhibitors, is being developed for the treatment of human autoimmune and auto-inflammatory diseases, such as systemic lupus erythematosus (SLE), scleroderma, psoriasis, Aicardi Goutieres syndrome (AGS), Sjogren's syndrome, rheumatoid arthritis, inflammatory bowel diseases, multiple sclerosis, diabetes, cardiovascular, cancer, and neurodegenerative diseases. However, a solid form of Compound A had not been identified during initial development, and a solid form would be important to fully investigate the compound's biological properties and potentially develop the compound as a therapeutic agent.

During drug development of an active pharmaceutical ingredient (API), the physical form of the API or a salt thereof can impact the physical properties of the drug candidate in the formulation. Many active pharmaceutical ingredients can exist in more than one solid form, including crystalline polymorphic forms. Moreover, a preferred form may also exist as a free base, free acid, or pharmaceutically acceptable salt. Therefore, the identification of pharmaceutically acceptable salt forms, as well as free base or free acid forms, for manufacturing represents an important step in the development of a drug candidate.

With regard to salts, certain compounds with ionizable functional groups readily form well-behaved solid form salts. However, other compounds, particularly those with both Brønsted-Lowry acidic and basic functional groups, often form salts with difficulty or in unpredictable ways, with no clear pattern for stability or the formation of solid forms. Compound A has both Brønsted-Lowry acidic and basic functional groups.

In identifying a candidate solid form for drug development, the candidate solid form is often one that possesses an unpredictable physical property. A particular solid form (e.g., crystalline or semi-solid or amorphous) may be preferrable because of ease of preparation, stability, etc. On the other hand, a different crystalline solid may be preferred for greater solubility and/or superior pharmacokinetics. Thus, while pharmaceutically acceptable salts of the active ingredient may provide increased solubility, the development of a particular pharmaceutical form, such as a crystalline solid of the salt, may be necessary to achieve a clinical pharmaceutical formulation.

### 2. SUMMARY

The present disclosure relates to solid forms of Compound A, pharmaceutically acceptable salts of Compound A, solid forms of pharmaceutically acceptable salts of Compound A, pharmaceutical compositions of the salts and the solid forms, methods of preparing the salts and the solid forms, and methods of using the salts and the solid forms.

In one aspect, embodiments herein relate to solid forms of Compound A:

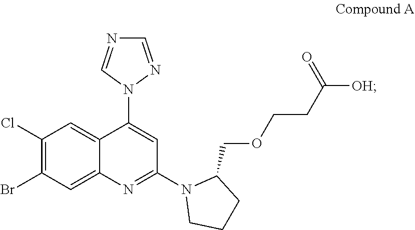 [SMILES: O=C(O)CCOC[C@@H]1CCCN1c1cc(-n2cncn2)c2cc(Cl)c(Br)cc2n1]

In another aspect, embodiments herein relate to a pharmaceutically acceptable salt of Compound A:

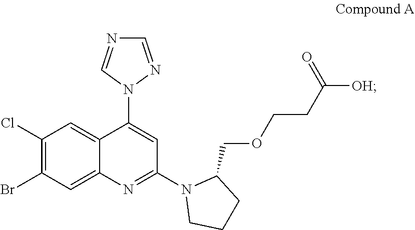 [SMILES: O=C(O)CCOC[C@@H]1CCCN1c1cc(-n2cncn2)c2cc(Cl)c(Br)cc2n1]

wherein the pharmaceutically acceptable salt is selected from hydrochloride salt, sulfate, napadisylate, esylate, mesylate, napsylate, besylate, sodium salt, potassium salt, L-arginine salt, choline salt, L-lysine salt, t-butylamine salt, meglumine salt, and tris(hydroxymethyl)aminomethane salt.

In certain aspects, embodiments herein relate to a pharmaceutical composition comprising an effective amount of a compound as contemplated herein and a pharmaceutically acceptable excipient.

In further aspects, embodiments herein relate to a method of antagonizing cyclic GMP-AMP synthase (cGAS) in a patient in need thereof, comprising administering an effective amount of a compound as contemplated herein or a pharmaceutical composition thereof.

In still further aspects, embodiments herein relate to a method of treating an inflammatory, allergic, autoimmune, or neurodegenerative disease in a patient in need thereof, comprising administering an effective amount of a compound as contemplated herein or a pharmaceutical composition thereof.

In another aspect, embodiments herein relate to use of a compound as contemplated herein or a pharmaceutical composition thereof in the manufacture of a medicament for antagonizing cyclic GMP-AMP synthase (cGAS) in a patient in need thereof.

In still another aspect, embodiments herein relate to use of a compound as contemplated herein or a pharmaceutical composition thereof in the manufacture of a medicament for treating an inflammatory, allergic, autoimmune, or neurodegenerative disease in a patient in need thereof.

In an aspect, embodiments herein relate to a compound as contemplated herein or a pharmaceutical composition thereof for use in therapy, such as for treating an inflammatory, allergic, autoimmune, cardiovascular, or neurodegenerative disease in a patient, and/or for antagonizing cyclic GMP-AMP synthase (cGAS) in a patient.

In particular, the present disclosure provides quinoline cGAS antagonist compounds, which find utility as inhibitors of cGAS. An advantage of the compounds provided herein is that a broad range of pharmacological activities is possible, consistent with the inhibition of cGAS. In addition, the disclosure provides methods of using the solid forms and compositions thereof described herein for the treatment of inflammatory, allergic, autoimmune, and infectious diseases. The solid forms and compositions thereof can also be used for the treatment of senescence- or age-related diseases, such as neurodegenerative diseases, cardiovascular diseases, liver and renal diseases, cancer, and premature aging.

### 3. BRIEF DESCRIPTION OF THE DRAWINGS

These and other features, aspects, and advantages of the present disclosure will become better understood with regard to the following description, and accompanying drawings, where:

FIG. 1 illustrates the XRPD pattern of the starting material, Compound A.

FIG. 2 illustrates the TGA/mDSC curves of the starting material, Compound A.

FIG. 3 illustrates the ¹H NMR of the starting material, Compound A.

FIG. 4 illustrates the PLM image of the starting material, Compound A.

FIG. 5 illustrates the XRPD pattern overlay of the isolation of Compound A HCl salt Type B.

FIG. 6 illustrates the XRPD pattern of the stress test results of Compound A esylate.

FIG. 7 illustrates the XRPD of Compound A freeform Type B.

FIG. 8 illustrates the TGA/DSC curves of Compound A freeform Type B.

FIG. 9 illustrates the ¹H NMR of Compound A freeform Type B.

FIG. 10 illustrates the PLM image of Compound A freeform Type B.

FIG. 11 illustrates the XRPD pattern of Compound A esylate.

FIG. 12 illustrates the TGA/DSC curves of Compound A esylate.

FIG. 13 illustrates the ¹H NMR of Compound A esylate.

FIG. 14 illustrates the PLM image of Compound A esylate.

FIG. 15 illustrates the XRPD pattern of Compound A t-butylamine.

FIG. 16 illustrates the TGA/DSC curves of Compound A t-butylamine.

FIG. 17 illustrates the ¹H NMR of Compound A t-butylamine.

FIG. 18 illustrates the PLM image of Compound A t-butylamine.

FIG. 19 illustrates the XRPD of Compound A tris salt.

FIG. 20 illustrates the TGA/DSC curves of Compound A tris salt.

FIG. 21 illustrates the ¹H NMR of Compound A tris salt.

FIG. 22 illustrates the PLM image of Compound A tris salt.

FIG. 23 illustrates the DVS plot of Compound A freeform Type B.

FIG. 24 illustrates the XRPD overlay of Compound A freeform Type B before and after DVS.

FIG. 25 illustrates the DVS plot of Compound A esylate.

FIG. 26 illustrates the XRPD overlay of Compound A esylate before and after DVS.

FIG. 27 illustrates the DVS plot of Compound A t-butylamine salt.

FIG. 28 illustrates the XRPD overlay of Compound A t-butylamine salt before and after DVS.

FIG. 29 illustrates the DVS plot of Compound A tris salt.

FIG. 30 illustrates the XRPD overlay of Compound A tris salt before and after DVS.

FIGS. 31A-31D illustrate the kinetic solubility curves of Compound A freeform B in various media. FIG. 31A represents the kinetic solubility curve in water at 37° C. FIG. 31B represents the kinetic solubility in fasted state simulated intestinal fluid (FaSSIF) at 37° C. FIG. 31C represents the kinetic solubility in fed state simulated intestinal fluid (FeSSIF) at 37° C. FIG. 31D represents the kinetic solubility in simulated gastric fluid (SGF) at 37° C.

FIGS. 32A-32D illustrate the kinetic solubility curves of Compound A esylate in various media. FIG. 32A represents the kinetic solubility curve in water at 37° C. FIG. 32B represents the kinetic solubility in FaSSIF at 37° C. FIG. 32C represents the kinetic solubility in FeSSIF at 37° C. FIG. 32D represents the kinetic solubility in SGF at 37° C.

FIGS. 33A-33D illustrate the kinetic solubility curves of Compound A tris salt in various media. FIG. 33A represents the kinetic solubility curve in water at 37° C. FIG. 33B represents the kinetic solubility in FaSSIF at 37° C. FIG. 33B represents the kinetic solubility in FaSSIF at 37° C. FIG. 33C represents the kinetic solubility in FeSSIF at 37° C. FIG. 33D represents the kinetic solubility in SGF at 37° C.

FIGS. 34A-34D illustrate the kinetic solubility curves of Compound A t-butylamine salt in various media. FIG. 34A represents the kinetic solubility curve in water at 37° C. FIG. 34B represents the kinetic solubility in FaSSIF at 37° C. FIG. 34B represents the kinetic solubility in FaSSIF at 37° C. FIG. 34C represents the kinetic solubility in FeSSIF at 37° C. FIG. 34D represents the kinetic solubility in SGF at 37° C.

FIG. 35 illustrates the XRPD overlay of Compound A freeform Type B in water and bio-relevant media at 24 h.

FIG. 36 illustrates the XRPD overlay of Compound A t-butylamine salt in SGF and water at 24 h. The star indicates an additional peak.

FIG. 37 illustrates the XRPD overlay of Compound A tris salt in FeSSIF.

FIG. 38 illustrates the XRPD overlay of Compound A tris salt in SGF.

FIG. 39 illustrates the XRPD overlay of Compound A freeform Type B after stability evaluation.

FIG. 40 illustrates the XRPD overlay of Compound A esylate after stability evaluation.

FIG. 41 illustrates the XRPD overlay of Compound A t-butylamine salt after stability evaluation.

FIG. 42 illustrates the XRPD overlay of Compound A tris salt after stability evaluation.

FIG. 43 illustrates the XRPD pattern of Compound A HCl salt type A.

FIG. 44 illustrates the XRPD pattern of Compound A HCl salt type B.

FIG. 45 illustrates the XRPD pattern of Compound A HCl salt type C.

FIG. 46 illustrates the XRPD pattern of Compound A HCl salt type D.

FIG. 47 illustrates the XRPD pattern of Compound A freeform Type A.

FIG. 48 illustrates the XRPD pattern of Compound A napadisylate.

FIG. 49 illustrates the XRPD pattern of Compound A mesylate.

FIG. 50 illustrates the XRPD pattern of Compound A napsylate.

FIG. 51 illustrates the XRPD pattern of Compound A besylate salt.

FIG. 52 illustrates the XRPD pattern of Compound A sodium salt.

FIG. 53 illustrates the XRPD pattern of Compound A potassium salt.

FIG. 54 illustrates the XRPD pattern of Compound A L-arginine salt.

FIG. 55 illustrates the XRPD pattern of Compound A choline salt.

FIG. 56 illustrates the XRPD pattern of Compound A L-lysine salt.

FIG. 57 illustrates the XRPD pattern of Compound A meglumine salt.

FIG. 58 illustrates the XRPD pattern of Compound A sulfate.

FIG. 59 illustrates the XRPD pattern of Compound A tris salt.

### 4. DETAILED DESCRIPTION

In the following disclosure, certain specific details are set forth in order to provide a thorough understanding of various embodiments. However, one skilled in the art will understand that the methods and uses described herein may be practiced without these details. In other instances, well-known structures have not been shown or described in detail to avoid unnecessarily obscuring descriptions of the embodiments. Unless the context requires otherwise, throughout the specification and claims which follow, the word “comprise” and variations thereof, such as, “comprises” and “comprising” are to be construed in an open, inclusive sense, that is, as “including, but not limited to.” Further, headings provided herein are for convenience only and do not interpret the scope or meaning of the claimed invention.

Reference throughout this specification to “one embodiment” or “an embodiment” means that a particular feature, structure, or characteristic described in connection with the embodiment is included in at least one embodiment. Thus, the appearances of the phrases “in one embodiment” or “in an embodiment” in various places throughout this specification are not necessarily all referring to the same embodiment. Furthermore, the particular features, structures, or characteristics may be combined in any suitable manner in one or more embodiments. Also, as used in this specification and the appended claims, the singular forms “a,” “an,” and “the” include plural referents unless the content clearly dictates otherwise. It should also be noted that the term “or” is generally employed in its sense including “and/or” unless the content clearly dictates otherwise.

### 4.1. Definitions

Compounds of the present disclosure include those described generally herein, and are further illustrated by the classes, subclasses, and species disclosed herein. As used herein, the following definitions shall apply unless otherwise indicated. For purposes of this disclosure, the chemical elements are identified in accordance with the Periodic Table of the Elements, CAS version, Handbook of Chemistry and Physics, 75^{th} Ed.

Compounds of the present disclosure, freeform and salts thereof, may exist in multiple tautomeric forms, in which hydrogen atoms are transposed to other parts of the molecules and the chemical bonds between the atoms of the molecules are consequently rearranged. It should be understood that all tautomeric forms, insofar as they may exist, are included within the disclosed embodiments.

Values may be expressed as approximations by the use of the antecedent “about” or as a range with the use of “±”. When values are expressed as approximations, by use of the antecedent “about,” it will be understood that the particular value forms another embodiment. As used herein, “about X” (where X is a numerical value) preferably refers to ±10% of the recited value, inclusive. For example, the phrase “about 8” refers to a value of 7.2 to 8.8, inclusive; as another example, the phrase “about 8%” refers to a value of 7.2% to 8.8%, inclusive. When values are expressed as “X±0.2 degrees 2θ” or “X±2° C.”, it is understood that the particular value is a range as recited or claimed. For example, the value “8.0±0.2 degrees 2θ” refers to a value of 7.8 degrees 2θ to 8.2 degrees 2θ, inclusive. Where present, all ranges are inclusive and combinable.

All ranges disclosed herein are inclusive of the recited endpoint and independently combinable (for example, the range of “from 100 mg to 200 mg” is inclusive of the endpoints, 100 mg and 200 mg, and all the intermediate values). The endpoints of the ranges and any values disclosed herein are not limited to the precise range or value; they are sufficiently imprecise to include values approximating these ranges and/or values.

Unless otherwise indicated, all chiral (enantiomeric and diastereomeric) and racemic forms are within the scope of the disclosed embodiments. Many geometric isomers of C═C double bonds, C═N double bonds, ring systems, and the like can also be present in the compounds, and all such stable isomers are contemplated in the present disclosure. Cis- and trans-(or E- and Z-) geometric isomers of the compounds of the present disclosure are described and may be isolated as a mixture of isomers or as separated isomeric forms.

If there is a discrepancy between a depicted structure and a name given to that structure, then the depicted structure controls. Additionally, if the stereochemistry of a structure or a portion of a structure is not indicated with, for example, bold or dashed lines, the structure or portion of the structure is to be interpreted as encompassing all stereoisomers of it. In some cases, however, where more than one chiral center exists, the structures and names may be represented as single enantiomers to help describe the relative stereochemistry. It is understood to those skilled in the art of organic synthesis if and when the compounds are prepared as single enantiomers from the methods used to prepare them.

The term “API” refers to the active pharmaceutical ingredient. As used herein, API refers to “Compound A” or (S)-3-((1-(7-bromo-6-chloro-4-(1H-1,2,4-triazol-1-yl)quinolin-2-yl)pyrrolidin-2-yl)methoxy)propanoic acid:

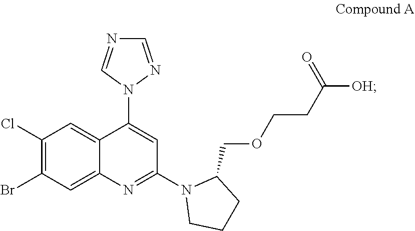 [SMILES: O=C(O)CCOC[C@@H]1CCCN1c1cc(-n2cncn2)c2cc(Cl)c(Br)cc2n1]

A solid form of Compound A, such as a crystalline form, may be referred to herein as being characterized by graphical data “as depicted in” or “as substantially depicted in” a figure. Such data include, for example, X-ray powder diffractograms, DSC thermograms, or NMR spectrums. As is well-known in the art, such graphical data potentially provides additional technical information to further define the respective solid-state form (a so-called “fingerprint”) which cannot necessarily be described by reference to numerical values or peak positions alone. The skilled person will understand that such graphical representations of data may be subject to small variations, e.g., in peak relative intensities and peak positions due to certain factors such as, but not limited to, variations in instrument response and variations in sample concentration and purity, which are well known to the skilled person. Further, the skilled person would readily be capable of comparing the graphical data in the Figures herein with graphical data generated for an unknown crystalline form.

The term “chemically stable,” as used herein, means that the chemical structure of a particular compound, does not change into another compound (e.g., decompose) when subjected to specified conditions, e.g., room temperature ambient humidity or 40° C./75% relative humidity, for a specified period of time, e.g., 1 day, 2 days, 3 days, 1 week, 2 weeks, 1 month, 2 months, 3 months, 6 months, 12 months, 18 months, 24 months, or longer. In some embodiments, less than 25% of the form of a particular compound changes into one or more other compounds when subjected to specified conditions. In some embodiments, less than about 20%, less than about 15%, less than about 10%, less than about 5%, less than about 3%, less than about 1%, less than about 0.5% of the form of a particular compound changes into one or more other compounds when subjected to specified conditions. In some embodiments, no detectable amount of the form of a particular compound changes into one or more different physical forms of that particular compound.

As used herein, “differential scanning calorimetry” or “DSC” refers to an analytical method that measures the amount of heat required to increase the temperature of a sample and is measured as a function of temperature. Such measurements provide quantitative and qualitative information about physical and chemical changes that involve endothermic or exothermic processes, or changes in heat capacity. A DSC thermogram can be depicted as a plot of heat flow versus temperature and can show both positive and negative heat flow on the same plot. A DSC plot can show one or more endotherms, exotherms, or both. The values of DSC exotherms and endotherms are expressed in degrees Celsius (° C.), which represent the temperature of the onset of the exotherm or endotherm and allows for and includes an appropriate margin of error, which can be represented by the symbol “+.” For example, a DSC endotherm with a value of “228.03±2° C.” can refer to a range from 228.03+2° C. to 228.03-2° C., that is, 230.03° C. to 226.03° C. Depending on the sample preparation technique, the scan rate, the calibration technique applied to the instrument, human operation variation, the nature of the particular instrument being used, and other factors known to the art, the appropriate margin of error for a DSC endotherm or exotherm onset and peak temperature can be about ±5° C., about ±4° C., about ±3° C., about ±2° C., about ±2° C., about ±0.5° C., about ±0.2° C., about ±0.1° C. or less. In some cases, a DSC endotherm can represent a melting temperature, and a DSC exotherm can represent a freezing temperature, although this is not always the case. In view of these variabilities, DSC results are sometimes reported as a range. In embodiments, modulated DSC (mDSC) is employed. Modulated DSC refers to a method that uses sinusoidal temperature oscillation, which separates the total heat flow into reversing and non-reversing components.

The term “effective amount” refers to an amount of a compound or other active ingredient sufficient to provide a therapeutic or prophylactic benefit in the treatment or prevention of a disease or to delay or minimize symptoms associated with a disease. Further, a therapeutically effective amount with respect to a compound means that amount of therapeutic agent alone, or in combination with other therapies, that provides a therapeutic benefit in the treatment or prevention of a disease. Used in connection with a compound, the term can encompass an amount that improves overall therapy, reduces or avoids symptoms or causes of disease, or enhances the therapeutic efficacy or synergies with another therapeutic agent.

As used herein, “equilibrating,” when used in reference to conditions for forming a polymorph of a compound, refers to a physical process that results in thermodynamic equilibrium.

As used herein, “high-pressure liquid chromatography” or “HPLC” refers to an analytical method used to separate and quantify analytes. The technique relies on high pressure pumps that deliver a mixture of solvents, known as the mobile phase, which carries the sample mixture and flows through the system over the stationary phase, which is made of adsorbent material. The technique is a separation technique based on adsorption and partitioning.

As used herein, “hydrate” refers to a solvate wherein the one or more solvents comprises water.

As used herein, “non-solid” refers to a substance that does not possess a form. In embodiments, a non-solid includes, but is not limited to liquids, gels, creams, ointments, suspensions, emulsions, and aerosols. Non-solid pharmaceutical formulations are typically used if they possess properties such as ease of administration, rapid absorption, and ability to deliver active ingredients that are more bioavailable compared to the solid form such as tablets or capsules.

As used herein, “nuclear magnetic resonance spectroscopy” also referred to as “NMR” is an analytical technique used in the field of organic chemistry for structure elucidation. The technique applies a magnetic field to an atomic nucleus and radio frequency pulses to characterize the resonant frequence of that atomic nucleus according to its chemical or environmental surroundings.

A “patient” or “subject” includes an animal, such as a human, cow, horse, sheep, lamb, pig, chicken, turkey, quail, cat, dog, mouse, rat, rabbit, or guinea pig. The animal can be a mammal such as a non-primate and a primate (e.g., monkey and human). In one embodiment, a patient is a human, such as a human infant, child, adolescent, or adult.

As used herein, the term “pharmaceutically acceptable carrier” means a pharmaceutically acceptable material, composition or carrier, such as a liquid or solid, stabilizer, dispersing agent, suspending agent, diluent, excipient, thickening agent, solvent or encapsulating material, involved in carrying or transporting a salt of the compound useful within the disclosed embodiments or to the subject such that it may perform its intended function. Each carrier must be “acceptable” in the sense of being compatible with the other ingredients of the formulation, including the salt useful within the disclosed embodiments, and not injurious to the subject. Some examples of materials that may serve as pharmaceutically acceptable carriers include: sugars, such as lactose, glucose and sucrose; starches, such as corn starch and potato starch; cellulose, and its derivatives, such as sodium carboxymethyl cellulose, ethyl cellulose and cellulose acetate; powdered tragacanth; malt; gelatin; talc; excipients, such as cocoa butter and suppository waxes; oils, such as peanut oil, cottonseed oil, safflower oil, sesame oil, olive oil, corn oil and soybean oil; glycols, such as propylene glycol; polyols, such as glycerin, sorbitol, mannitol and polyethylene glycol; esters, such as ethyl oleate and ethyl laurate; agar; buffering agents, such as magnesium hydroxide and aluminum hydroxide; surface active agents; alginic acid; pyrogen-free water; isotonic saline; Ringer's solution; ethyl alcohol; phosphate buffer solutions; and other non-toxic compatible substances employed in pharmaceutical formulations.

Proper formulation is dependent upon the route of administration chosen. Additional details about suitable excipients for pharmaceutical compositions described herein may be found, for example, in Remington: The Science and Practice of Pharmacy, Volume I and Volume II, Twenty-Second Edition, Loyd V. Allen, Jr., editor (Philadelphia, PA: Pharmaceutical Press, 2012); Excipient Development for Pharmaceutical, Biotechnology, and Dmg Delivery Systems, Ashok Katdare and Mahesh V. Chaubal, editors (Boca Raton, FL: CRC Press, 2006); and Pharmaceutical Dosage Forms and Drug Delivery Systems, Seventh Ed. (Lippincott Williams & Wilkins 1999), herein incorporated by reference for such disclosure.

As used herein, “pharmaceutically acceptable salts” refer to derivatives of the disclosed compounds wherein the parent compound is modified by making acid or base salts thereof. Examples of pharmaceutically acceptable salts include, but are not limited to, mineral or organic acid salts of basic groups such as amines; and alkali or organic salts of acidic groups such as carboxylic acids. The pharmaceutically acceptable salts include the conventional non-toxic salts or the quaternary ammonium salts of the parent compound formed, for example, from non-toxic inorganic or organic acids. For example, such conventional non-toxic salts include those derived from inorganic acids such as hydrochloric, hydrobromic, sulfuric, sulfamic, phosphoric, and nitric; and the salts prepared from organic acids such as acetic, propionic, succinic, glycolic, stearic, lactic, malic, tartaric, citric, ascorbic, pamoic, maleic, hydroxymaleic, phenylacetic, glutamic, benzoic, salicylic, sulfanilic, 2-acetoxybenzoic, fumaric, toluenesulfonic, methanesulfonic, ethane disulfonic, oxalic, and isethionic.

As used herein “pharmaceutical composition,” refers to a mixture of Compound A or a pharmaceutically acceptable salt thereof, including solid forms of these, as described herein, with other chemical components, such as carriers, stabilizers, diluents, dispersing agents, suspending agents, thickening agents, and/or excipients. The pharmaceutical composition facilitates administration of the compound to an organism.

The term “physically stable,” as used herein, means that a particular free base or salt form does not change into one or more different physical forms (e.g., different solid forms as measured by XRPD, DSC, etc.) when subjected to specified conditions, e.g., room temperature ambient humidity or 40° C./75% relative humidity, for a specified period of time, e.g., 1 day, 2 days, 3 days, 1 week, 2 weeks, 1 month, 2 months, 3 months, 6 months, 12 months, 18 months, 24 months, or longer. In some embodiments, less than 25% of the form of a compound changes into one or more different physical forms when subjected to specified conditions. In some embodiments, less than about 20%, less than about 15%, less than about 10%, less than about 5%, less than about 3%, less than about 1%, less than about 0.5% of the form of a particular compound changes into one or more different physical forms of that particular compound when subjected to specified conditions. In some embodiments, no detectable amount of the particular form of a compound changes into one or more different physical forms of the compound.

As used herein, “polarized light microscopy” or “PLM” is a technique for determining if a sample is crystalline or not. The technique uses polarized light and is used to assess amorphous/crystalline content to identify those samples which should be further characterized by XRPD. PLM can also distinguish different crystalline polymorphs based on shape, morphology, size and other optical characteristics.

As used herein, the term “polymorph” refers to a crystal form of a compound. The term “polymorph,” “crystal form,” “Form,” or “Type,” followed by an alphabet identifier are used interchangeably. Such crystal forms may be identified by, inter alia, X-ray diffraction patterns.

The symbols “R” and “S” represent the configuration of substituents around a chiral carbon atom(s). The isomeric descriptors “R” and “S” are used as described herein for indicating atom configuration(s) relative to a core molecule and are intended to be used as defined in the literature (IUPAC Recommendations 1996, Pure and Applied Chemistry, 68:2193-2222 (1996)).

As used herein, “seeding,” refers to the process of using a small quantity of an existing crystal form to aid in crystallization of more of that form from a solution or suspension of the compound of interest.

As used herein “slurrying,” when used in reference to conditions for forming a polymorph of a compound, refers to physical process that leads to a thermodynamic equilibrium in a suspension, which is typically solid particles/material dispersed in a liquid, by stirring the suspension with a suitable stirring apparatus.

As used herein, “solid forms” refer to polymorphic forms of a chemical compound. A solid form can exist as a crystalline solid or an amorphous solid. A chemical compound may exist as multiple solid forms and will be identified by “Form” or “Type” followed by an alphabet identifier. In embodiments, only one solid form may be identified or isolated.

As used herein, “solvate” refers to a crystalline form of a compound or mixture of compounds that comprises one or more solvents in its crystal lattice.

The term “stereoisomer” refers to isomers of identical constitution that differ in the arrangement of their atoms in space. Enantiomers and diastereomers are examples of stereoisomers. The term “enantiomer” refers to one of a pair of molecular species that are mirror images of each other and are not superimposable. The term “diastereomer” refers to stereoisomers that are not mirror images.

As used herein, “substantially free” refers to a substance that contains less than 10% of impurities, thereby achieving a purity level of at least 90% or greater. In embodiments, substantially free refers to a substance that contains less than 9% of impurities, less than 8% of impurities, less than 7% of impurities, less than 6% of impurities, less than 5% of impurities, less than 4% of impurities, less than 3% of impurities, less than 2% of impurities, less than 1% of impurities, or less than 0.5% of impurities. As contemplated herein, the impurities may refer to other solid forms. In embodiments, the impurities may refer to other compounds such as a derivative.

As used herein, “suspension equilibrating,” when used in reference to conditions for forming a polymorph of a compound, refers to a physical process that leads to a thermodynamic equilibrium in a suspension, which is typically solid particles/material dispersed in a liquid.

In this description, the term “tautomer” or “tautomeric form” refers to structural isomers of different energies which are interconvertible via a low energy barrier. For example, proton tautomers (also known as prototropic tautomers) include interconversions via migration of a proton, such as keto-enol and imine-enamine isomerizations. Valence tautomers include interconversions by reorganization of some of the bonding electrons.

As used herein, “thermogravimetric analysis” or “TGA” refers to an analytical method for the characterization of solids that measures the change in the mass of a sample over time as the temperature changes (i.e., heated or cooled). The method quantifies, for example, the loss of water, loss of solvent, decarboxylation, pyrolysis, oxidation, and decomposition. An TGA plot can be a graph of weight percent and/or heat flow versus temperature. The weight gain or loss can be reversible or irreversible. The weight change is often reported as a weight percent (wt %), the value of which allows for and includes an appropriate margin of error that can be represented by the symbol “±.” For example, a weight change of about 2.0±0.5% can refer to a range from about 2+0.5%, that is, 2.5%, to about 2-0.05%, that is, about 1.5%. Depending on the sample preparation technique, the scan parameters, extent of sample drying pre or during analysis, the scan rate, the calibration technique applied to the instrument, human operation variation, the nature of the particular instrument being used, and other factors known to the art, the appropriate margin of error for a weight change by TGA can be about ±2%, about ±1%, about ±0.75%, about ±0.5%, about ±0.25%, or less. Among other things, TGA can be useful in determining whether a particular crystalline form is a solvate or hydrate, and if so, the degree of solvation or hydration. TGA is useful for this because, upon heating, the solvent or water in solvates or hydrates can evaporate causing a loss of the mass associated with the evaporated water. Thus, an TGA plot showing a small weight loss upon heating can be consistent with a salt that is neither a solvate nor a hydrate, whereas an TGA plot showing a significant weight loss can be consistent with a solvate or hydrate. TGA can also show decomposition related loss of mass form the sample.

The terms “treat,” “treating,” and “treatment” refer to the amelioration or eradication of a disease or symptoms associated with a disease. In embodiments, such terms refer to minimizing the spread or worsening of the disease resulting from the administration of one or more prophylactic or therapeutic agents to a patient with such a disease. In the context of the present disclosure the terms “treat,” “treating,” and “treatment” also refer to:
a. preventing the disease or condition from occurring in a mammal, in particular, when such mammal is predisposed to the condition but has not yet been diagnosed as having it;
b. inhibiting the disease or condition, i.e., arresting its development;
c. relieving the disease or condition, i.e., causing regression of the disease or condition; or
d. relieving the symptoms resulting from the disease or condition, i.e., relieving pain without addressing the underlying disease or condition. As used herein, the terms “disease” and “condition” may be used interchangeably or may be different in that the particular malady or condition may not have a known causative agent (so that etiology has not yet been worked out) and it is therefore not yet recognized as a disease but only as an undesirable condition or syndrome, wherein a more or less specific set of symptoms have been identified by clinicians.

As used herein, “X-ray powder diffraction” or “XRPD” refers to a technique used to characterize polycrystalline materials. The method measures X-rays scattered by the crystals as a function of the scattering angle. The intensities of X-Ray diffraction peaks provide information about the anisotropy of crystallite orientation and the microstructure relative to the surface. The method is unable to characterize amorphous materials such as glass due to the absence of crystalline peaks.

### 4.2. Pharmaceutically Acceptable Salt

Provided herein are pharmaceutically acceptable salts of a cGAS antagonist. Pharmaceutically acceptable salts refer to derivatives of the disclosed compounds wherein the parent compound is modified by making acid or base salts thereof. Examples of pharmaceutically acceptable salts include, but are not limited to, mineral or organic acid salts of basic groups such as amines; and alkali or organic salts of acidic groups such as carboxylic acids.

Salts contemplated herein, but not limited to, include those derived from inorganic acids and organic acids such as hydrochloric acid, sulfuric acid, maleic acid, phosphoric acid, fumaric acid, citric acid, naphthalene-1,5-disulfonic acid, ethane-1,2-disulfonic acid, ethanesulfonic acid, p-toluenesulfonic acid, methanesulfonic acid, naphthalene-2-sulfonic acid, benzenesulfonic acid, and oxalic acid. Salts contemplated herein, but are not limited to, include those derived from inorganic bases and organic bases such as sodium hydroxide, potassium hydroxide, magnesium hydroxide, L-arginine, calcium hydroxide, choline, L-lysine, t-butylamine, ammonia, N-methyl glucamine (meglumine), deanol, tromethamine (tris), and L-proline.

As used herein, “hydrochloric acid” or “HC” refers to a strong acid used to form the hydrochloride salt. In embodiments, Compound A HCl salt (in a 1:1 molar charge to freeform ratio) is represented as follows:

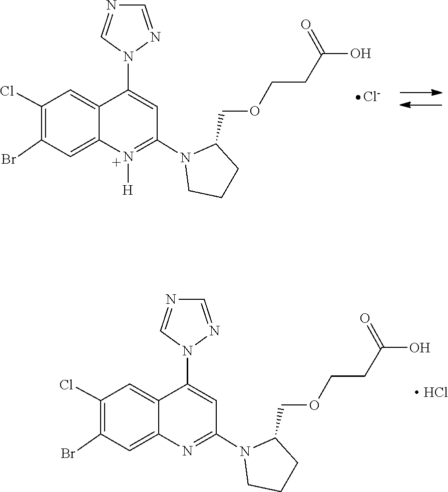

In embodiments, Compound A HCl salt (in a 2:1 molar charge to freeform ratio) is represented as follows:

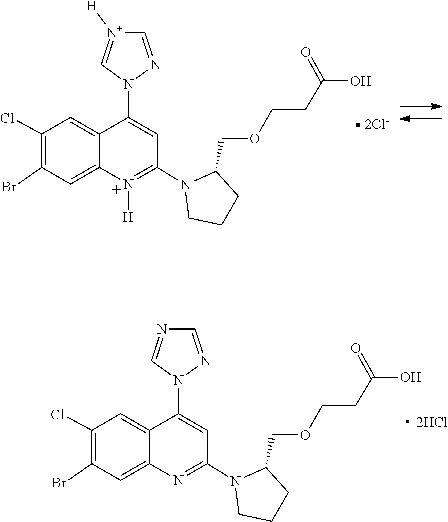

In embodiments, the solid form of Compound A HCl salt may be referred to as “Compound A HCl salt Type A,” “Compound A HCl salt Type B,” “Compound A HCl salt Type C,” or “Compound A HCl salt Type D.”

In embodiments, Compound A sulfate is represented as follows:

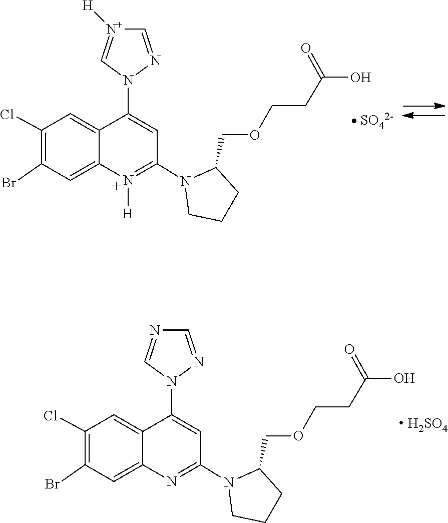

In embodiments, the solid form of Compound A sulfate may be referred to as “Compound A sulfate Type A.”

In embodiments, Compound A napadisylate is represented as follows:

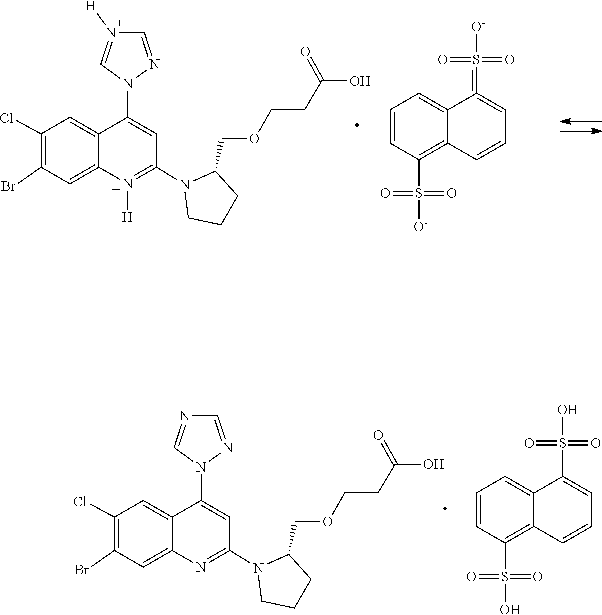

In embodiments, the solid form of Compound A napadisylate may be referred to as “Compound A napadisylate Type A.”

In embodiments, Compound A esylate is represented as follows:

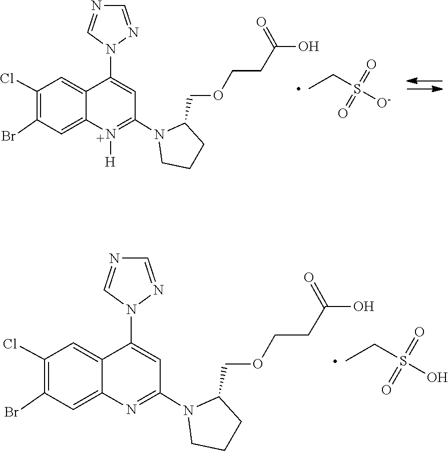

In embodiments, the solid form of Compound A esylate may be referred to as “Compound A esylate Type A.”

In embodiments, Compound A mesylate is represented as follows:

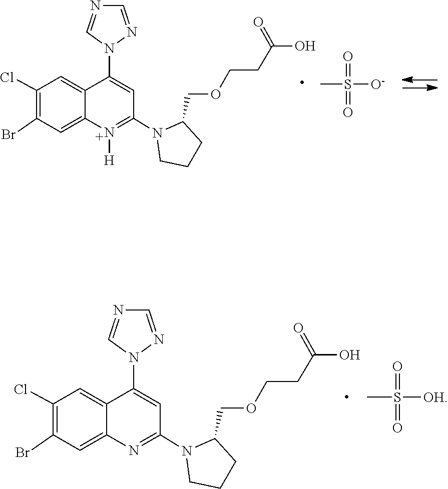

In embodiments, the solid form of Compound A mesylate may be referred to as “Compound A mesylate Type A.”

In embodiments, Compound A napsylate is represented as follows:

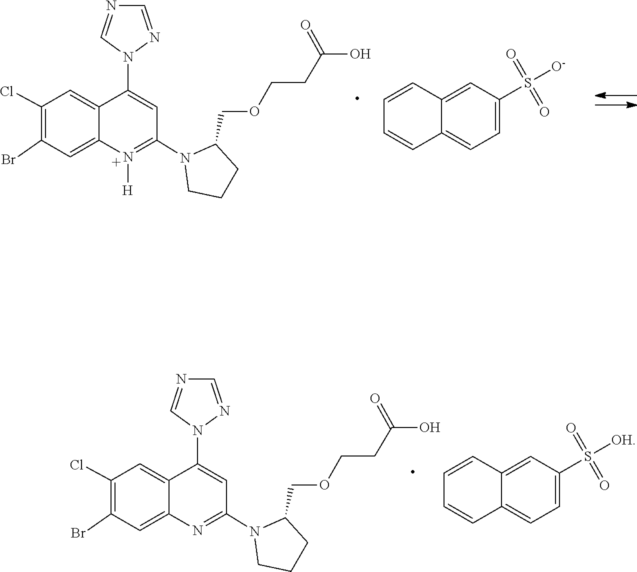

In embodiments, the solid form of Compound A napsylate may be referred to as “Compound A mesylate Type A.”

In embodiments, Compound A besylate is represented as follows:

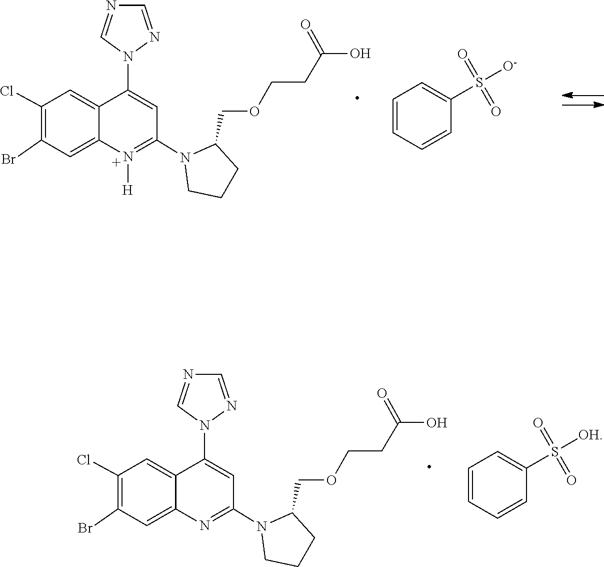

In embodiments, the solid form of Compound A besylate may be referred to as “Compound A besylate Type A.”

In embodiments, Compound A sodium salt is represented as follows:

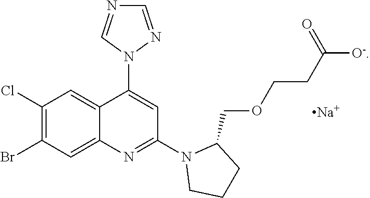

In embodiments, the solid form of Compound A sodium salt may be referred to as “Compound A sodium salt Type A.”

In embodiments, Compound A potassium salt is represented as follows:

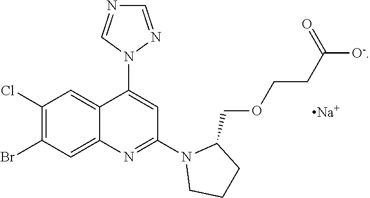

In embodiments, the solid form of Compound A potassium salt may be referred to as “Compound A potassium salt Type A.”

In embodiments, Compound A L-arginine salt is represented as follows:

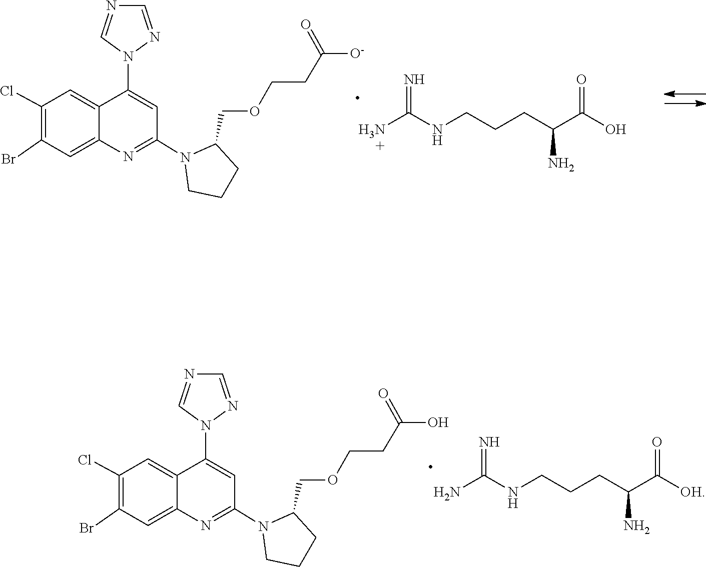 [SMILES: O=C([O-])CCOC[C@@H]1CCCN1c1cc(-n2cncn2)c2cc(Cl)c(Br)cc2n1 ; N=C(N)NCCC[C@H](N)C(=O)O ; O=C(O)CCOC[C@@H]1CCCN1c1cc(-n2cncn2)c2cc(Cl)c(Br)cc2n1 ; N=C(N)NCCC[C@H](N)C(=O)O]

In embodiments, the solid form of Compound A L-arginine salt may be referred to as “Compound A L-arginine salt Type A.”

In embodiments, Compound A choline is represented as follows:

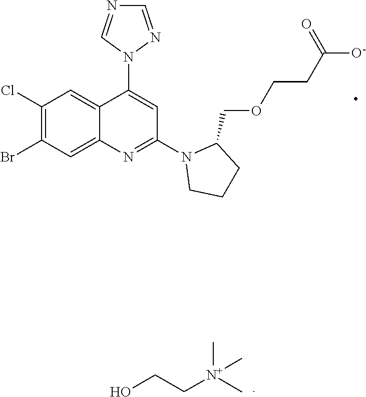

In embodiments, the solid form of Compound A choline may be referred to as “Compound A choline Type A.”

In embodiments, Compound A L-lysine salt is represented as follows:

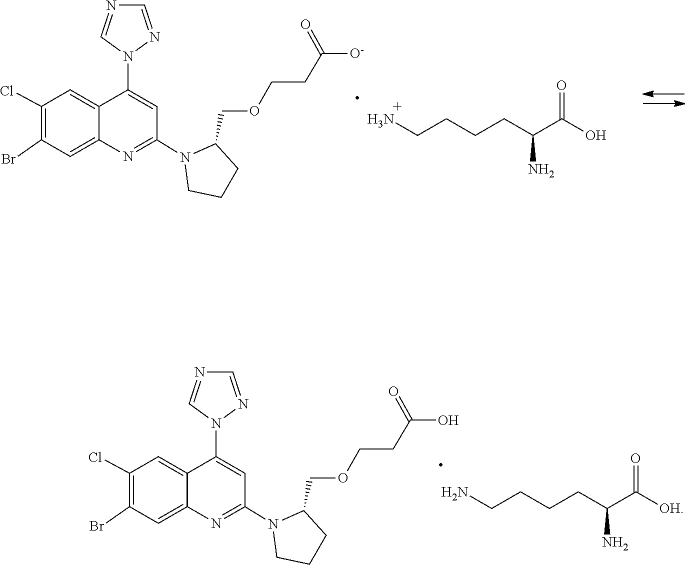 [SMILES: O=C([O-])CCOC[C@@H]1CCCN1c1cc(-n2cncn2)c2cc(Cl)c(Br)cc2n1 ; NCCCC[C@H](N)C(=O)O ; O=C(O)CCOC[C@@H]1CCCN1c1cc(-n2cncn2)c2cc(Cl)c(Br)cc2n1 ; NCCCC[C@H](N)C(=O)O]

In embodiments, the solid form of Compound A L-lysine salt may be referred to as “Compound A L-lysine salt Type A.”

In embodiments, Compound A t-butylamine salt is represented as follows:

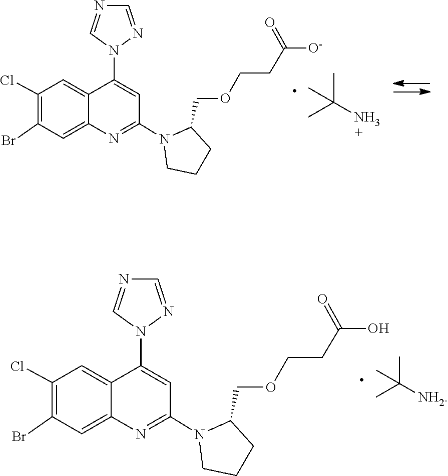

In embodiments, the solid form of Compound A t-butylamine salt may be referred to as “Compound A t-butylamine salt Type A.”

In embodiments, Compound A meglumine salt is represented as follows:

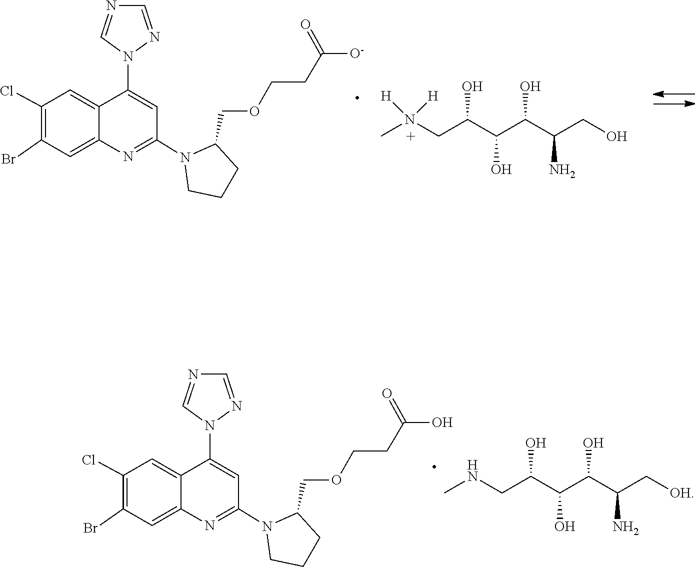

In embodiments, the solid form of Compound A meglumine salt may be referred to as “Compound A meglumine salt Type A.”

In embodiments, Compound A tris(hydroxymethyl)aminomethane salt also referred to as Compound A tris salt is represented as follows:

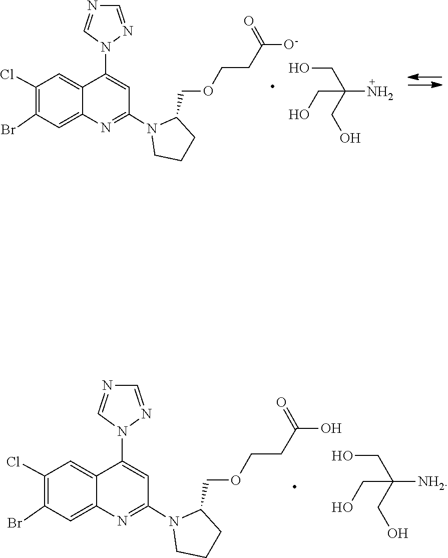

In embodiments, the solid form of Compound A tris salt is referred to as “Compound A tris salt Type A.”

### 4.3. Pharmaceutical Compositions

While it is possible that, for use in therapy, a provided compound may be administered as the raw chemical, it is possible to present the provided compound as the active ingredient in a pharmaceutical composition. Such compositions can be prepared in a manner well known in the pharmaceutical art and comprise at least one active compound. Accordingly in one embodiment, the disclosure further provides pharmaceutical compositions comprising a provided compound or a pharmaceutically acceptable salt thereof and one or more pharmaceutically acceptable excipients. The excipient(s) must be acceptable in the sense of being compatible with the other ingredients of the composition and not deleterious to the recipient thereof. In accordance with another aspect of the disclosure there is also provided a process for the preparation of a pharmaceutical composition including a provided compound or pharmaceutically acceptable salt thereof, with one or more pharmaceutically acceptable excipients. The pharmaceutical composition can be for use in the treatment and/or prophylaxis of any of the conditions described herein.

Generally, a provided compound is administered in a pharmaceutically effective amount. The amount of the compound actually administered will typically be determined by a physician, in the light of the relevant circumstances, including the condition to be treated, the chosen route of administration, the actual compound administered, the age, weight, and response of the individual patient, the severity of the patient's symptoms, and the like. Pharmaceutical compositions may be presented in unit dose forms containing a predetermined amount of active ingredient per unit dose. The term “unit dosage forms” refers to physically discrete units suitable as unitary dosages for human subjects and other mammals, each unit containing a predetermined quantity of active material calculated to produce the desired therapeutic effect, in association with a suitable pharmaceutical excipient, vehicle or carrier. Typical unit dosage forms include prefilled, premeasured ampules or syringes of the liquid compositions or pills, tablets, capsules or the like in the case of solid compositions.

Preferred unit dosage compositions are those containing a daily dose or sub-dose, or an appropriate fraction thereof, of an active ingredient. Such unit doses may therefore be administered once or more than once a day. Such pharmaceutical compositions may be prepared by any of the methods well known in the pharmacy art.

Pharmaceutical compositions may be adapted for administration by any appropriate route, for example by the oral (including buccal or sublingual), rectal, inhaled, intranasal, topical (including buccal, sublingual or transdermal), vaginal or parenteral (including subcutaneous, intramuscular, intravenous or intradermal) route. Such compositions may be prepared by any method known in the art of pharmacy, for example by bringing into association the active ingredient with the carrier(s) or excipient(s).

Pharmaceutical compositions adapted for oral administration may be presented as discrete units such as capsules or tablets; powders or granules; solutions or suspensions in aqueous or non-aqueous liquids; edible foams or whips; or oil-in-water liquid emulsions or water-in-oil liquid emulsions.

For instance, for oral administration in the form of a tablet or capsule, the active drug component can be combined with an oral, non-toxic pharmaceutically acceptable inert excipient such as ethanol, glycerol, water and the like. Powders are prepared by reducing the compound to a suitable fine size and mixing with a similarly prepared pharmaceutical excipient such as an edible carbohydrate, as, for example, starch or mannitol. Flavoring, preservative, dispersing and coloring agent can also be present.

Capsules are made by preparing a powder mixture, as described above, and filling formed gelatin sheaths. Excipients including glidants and lubricants such as colloidal silica, talc, magnesium stearate, calcium stearate or solid polyethylene glycol can be added to the powder mixture before the filling operation. A disintegrating or solubilizing agent such as agar-agar, calcium carbonate or sodium carbonate can also be added to improve the availability of the medicament when the capsule is ingested.

Moreover, when desired or necessary, excipients including suitable binders, glidants, lubricants, sweetening agents, flavors, disintegrating agents and coloring agents can also be incorporated into the mixture. Suitable binders include starch, gelatin, natural sugars such as glucose or beta-lactose, corn sweeteners, natural and synthetic gums such as acacia, tragacanth or sodium alginate, carboxymethylcellulose, polyethylene glycol, waxes and the like. Lubricants used in these dosage forms include sodium oleate, sodium stearate, magnesium stearate, sodium benzoate, sodium acetate, sodium chloride and the like. Disintegrators include, without limitation, starch, methyl cellulose, agar, bentonite, xanthan gum and the like. Tablets are formulated, for example, by preparing a powder mixture, granulating or slugging, adding a lubricant and disintegrant and pressing into tablets. A powder mixture is prepared by mixing a provided compound, suitably comminuted, with a diluent or base as described above, and optionally, with a binder such as carboxymethylcellulose, an alginate, gelatin, or polyvinyl pyrrolidone, a solution retardant such as paraffin, a resorption accelerator such as a quaternary salt and/or an absorption agent such as bentonite, kaolin or dicalcium phosphate. The powder mixture can be granulated by wetting with a binder such as syrup, starch paste, acacia mucilage or solutions of cellulosic or polymeric materials and forcing through a screen. As an alternative to granulating, the powder mixture can be run through the tablet machine and the result is imperfectly formed slugs broken into granules. The granules can be lubricated to prevent sticking to the tablet forming dies by means of the addition of stearic acid, a stearate salt, talc or mineral oil. The lubricated mixture is then compressed into tablets. A provided compound can also be combined with a free-flowing inert carrier and compressed into tablets directly without going through the granulating or slugging steps. A clear or opaque protective coating consisting of a sealing coat of shellac, a coating of sugar or polymeric material and a polish coating of wax can be provided. Dyestuffs can be added to these coatings to distinguish different unit dosages.

Oral fluids such as solution, suspensions, syrups and elixirs can be prepared in dosage unit form so that a given quantity contains a predetermined amount of the compound. Syrups can be prepared by dissolving the compound in a suitably flavored aqueous solution, while elixirs are prepared through the use of a non-toxic alcoholic vehicle. Suspensions can be formulated by dispersing the compound in a non-toxic vehicle. Solubilizers and emulsifiers such as ethoxylated isostearyl alcohols and polyoxy ethylene sorbitol ethers, preservatives, flavor additive such as peppermint oil or natural sweeteners or saccharin or other artificial sweeteners, and the like can also be added.

Where appropriate, dosage unit compositions for oral administration can be microencapsulated. The composition can also be prepared to prolong or sustain the release as for example by coating or embedding particulate material in polymers, wax or the like.

The compounds of the disclosure may also be administered in the form of liposome delivery systems, such as small unilamellar vesicles, large unilamellar vesicles and multilamellar vesicles. Liposomes can be formed from a variety of phospholipids, such as cholesterol, stearylamine or phosphatidylcholines. Pharmaceutical compositions adapted for transdermal administration may be presented as discrete patches intended to remain in intimate contact with the epidermis of the recipient for a prolonged period of time.

Pharmaceutical compositions adapted for topical administration may be formulated as ointments, creams, suspensions, lotions, powders, solutions, pastes, gels, sprays, aerosols or oils.

For treatments of the eye or other external tissues, for example mouth and skin, the compositions are preferably applied as a topical ointment or cream. When formulated in an ointment, the active ingredient may be employed with either a paraffinic or a water-miscible ointment base. Alternatively, the active ingredient may be formulated in a cream with an oil-in-water cream base or a water-in-oil base.

Pharmaceutical compositions adapted for topical administrations to the eye include eye drops wherein the active ingredient is dissolved or suspended in a suitable carrier, especially an aqueous solvent.

Pharmaceutical compositions adapted for topical administration in the mouth include lozenges, pastilles and mouth washes.

Pharmaceutical compositions adapted for rectal administration may be presented as suppositories or as enemas.

Dosage forms for nasal or inhaled administration may conveniently be formulated as aerosols, solutions, suspension drops, gels or dry powders.

Compositions for intranasal administration include aqueous compositions administered to the nose by drops or by pressurized pump. Suitable compositions contain water as the diluent or carrier for this purpose. Compositions for administration to the lung or nose may contain one or more excipients, for example one or more suspending agents, one or more preservatives, one or more surfactants, one or more tonicity adjusting agents, one or more co-solvents, and may include components to control the pH of the composition, for example a buffer system. Further, the compositions may contain other excipients such as antioxidants, for example sodium metabisulphite, and taste-masking agents. Compositions may also be administered to the nose or other regions of the respiratory tract by nebulization. Intranasal compositions may permit a provided compound or pharmaceutically acceptable salt thereof to be delivered to all areas of the nasal cavities (the target tissue) and further, may permit the provided compound or pharmaceutically acceptable salt thereof to remain in contact with the target tissue for longer periods of time. A suitable dosing regimen for intranasal compositions would be for the patient to inhale slowly through the nose subsequent to the nasal cavity being cleared. During inhalation, the composition would be administered to one nostril while the other is manually compressed. This procedure would then be repeated for the other nostril. Typically, one or two sprays per nostril would be administered by the above procedure one, two, or three times each day, ideally once daily. Of particular interest are intranasal compositions suitable for once-daily administration.

The suspending agent(s), if included, will typically be present in an amount of from 0.1 to 5% (w/w), such as from 1.5% to 2.4% (w/w), based on the total weight of the composition. Examples of pharmaceutically acceptable suspending agents include, but are not limited to, Avicef (microcrystalline cellulose and carboxymethylcellulose sodium), carboxymethylcellulose sodium, veegum, tragacanth, bentonite, methylcellulose, xanthan gum, carbopol and polyethylene glycols.

Compositions for administration to the lung or nose may contain one or more excipients may be protected from microbial or fungal contamination and growth by inclusion of one or more preservatives. Examples of pharmaceutically acceptable antimicrobial agents or preservatives include, but are not limited to, quaternary ammonium compounds (for example benzalkonium chloride, benzethonium chloride, cetrimide, cetylpyridinium chloride, lauralkonium chloride and myristyl picolinium chloride), mercurial agents (for example phenylmercuric nitrate, phenylmercuric acetate and thimerosal), alcoholic agents (for example chlorobutanol, phenylethyl alcohol and benzyl alcohol), antibacterial esters (for example esters of p-hydroxybenzoic acid), chelating agents such as disodium edetate (EDTA) and other antimicrobial agents such as chlorhexidine, chlorocresol, sorbic acid and its salts (such as potassium sorbate) and polymyxin. Examples of pharmaceutically acceptable antifungal agents or preservatives include, but are not limited to, sodium benzoate, sorbic acid, sodium propionate, methylparaben, ethylparaben, propylparaben, and butylparaben. The preservative(s), if included, may be present in an amount of from 0.001 to 1% (w/w), such as from 0.015% to 0.5% (w/w) based on the total weight of the composition. Compositions (for example wherein at least one compound is in suspension) may include one or more surfactants which functions to facilitate dissolution of the medicament particles in the aqueous phase of the composition. For example, the amount of surfactant used is an amount which will not cause foaming during mixing. Examples of pharmaceutically acceptable surfactants include fatty alcohols, esters, and ethers, such as polyoxyethylene (20) sorbitan monooleate (Polysorbate 80), macrogol ethers, and poloxamers. The surfactant may be present in an amount of between about 0.01 to 10% (w/w), such as from 0.01 to 0.75% (w/w), for example about 0.5% (w/w), based on the total weight of the composition.

One or more tonicity-adjusting agent(s) may be included to achieve tonicity with body fluids (e.g., fluids of the nasal cavity) resulting in reduced levels of irritancy. Examples of pharmaceutically acceptable tonicity-adjusting agents include, but are not limited to, sodium chloride, dextrose, xylitol, calcium chloride, glucose, glycerine, and sorbitol. A tonicity-adjusting agent, if present, may be included in an amount of from 0.1 to 10% (w/w), such as from 4.5 to 5.5% (w/w), for example about 5.0% (w/w), based on the total weight of the composition.

The compositions of the disclosure may be buffered by the addition of suitable buffering agents such as sodium citrate, citric acid, trometamol, phosphates such as disodium phosphate (e.g., dodecahydrate, heptahydrate, dihydrate and anhydrous forms), or sodium phosphate and mixtures thereof.

A buffering agent, if present, may be included in an amount of from 0.1 to 5% (w/w), for example 1 to 3% (w/w) based on the total weight of the composition.

Examples of taste-masking agents include sucralose, sucrose, saccharin or a salt thereof, fructose, dextrose, glycerol, corn syrup, aspartame, acesulfame-K, xylitol, sorbitol, erythritol, ammonium glycyrrhizinate, thaumatin, neotame, mannitol, menthol, eucalyptus oil, camphor, a natural flavoring agent, an artificial flavoring agent, and combinations thereof.

One or more co-solvent may be included to aid solubility of the medicament compound(s) and/or other excipients. Examples of pharmaceutically acceptable co-solvents include, but are not limited to, propylene glycol, dipropylene glycol, ethylene glycol, glycerol, ethanol, polyethylene glycols (for example PEG300 or PEG400), and methanol. In one embodiment, the co-solvent is propylene glycol.

Co-solvent(s), if present, may be included in an amount of from 0.05 to 30% (w/w), such as from 1 to 25% (w/w), for example from 1 to 10% (w/w) based on the total weight of the composition.

Compositions for inhaled administration include aqueous, organic or aqueous/organic mixtures, dry powder or crystalline compositions administered to the respiratory tract by pressurized pump or inhaler, for example, reservoir dry powder inhalers, unit-dose dry powder inhalers, pre-metered multi-dose dry powder inhalers, nasal inhalers or pressurized aerosol inhalers, nebulizers or insufflators. Suitable compositions contain water as the diluent or carrier for this purpose and may be provided with conventional excipients such as buffering agents, tonicity modifying agents and the like. Aqueous compositions may also be administered to the nose and other regions of the respiratory tract by nebulization. Such compositions may be aqueous solutions or suspensions or aerosols delivered from pressurized packs, such as a metered dose inhaler, with the use of a suitable liquefied propellant.

Compositions for administration topically to the nose (for example, for the treatment of rhinitis) or to the lung, include pressurized aerosol compositions and aqueous compositions delivered to the nasal cavities by pressurized pump. Compositions which are non-pressurized and are suitable for administration topically to the nasal cavity are of particular interest. Suitable compositions contain water as the diluent or carrier for this purpose. Aqueous compositions for administration to the lung or nose may be provided with conventional excipients such as buffering agents, tonicity-modifying agents and the like. Aqueous compositions may also be administered to the nose by nebulization.

A fluid dispenser may typically be used to deliver a fluid composition to the nasal cavities. The fluid composition may be aqueous or non-aqueous, but typically aqueous. Such a fluid dispenser may have a dispensing nozzle or dispensing orifice through which a metered dose of the fluid composition is dispensed upon the application of a user-applied force to a pump mechanism of the fluid dispenser. Such fluid dispensers are generally provided with a reservoir of multiple metered doses of the fluid composition, the doses being dispensable upon sequential pump actuations. The dispensing nozzle or orifice may be configured for insertion into the nostrils of the user for spray dispensing of the fluid composition into the nasal cavity.

Dry powder compositions for topical delivery to the lung by inhalation may, for example, be presented in capsules and cartridges of for example gelatin, or blisters of for example laminated aluminum foil, for use in an inhaler or insufflator. Powder blend compositions generally contain a powder mix for inhalation of a provided compound or pharmaceutically acceptable salt thereof and a suitable powder base (carrier/diluent/excipient substance) such as mono-, di-, or polysaccharides (e.g., lactose or starch). Dry powder compositions may also include, in addition to the drug and carrier, a further excipient (e.g., a ternary agent such as a sugar ester for example cellobiose octaacetate, calcium stearate, or magnesium stearate.

Pharmaceutical compositions adapted for parental administration include aqueous and nonaqueous sterile injection solutions which may contain antioxidants, buffers, bacteriostats and solutes which render the composition isotonic with the blood of the intended recipient; and aqueous and non-aqueous sterile suspensions which may include suspending agents and thickening agents. The compositions may be presented in unit-dose or multi-dose containers, for example sealed ampoules and vials, and may be stored in a freeze-dried (e.g., lyophilized) condition requiring only the addition of the sterile liquid carrier, for example water for injections, immediately prior to use. Extemporaneous injection solutions and suspensions may be prepared from sterile powders, granules, and tablets.

It should be understood that in addition to the ingredients particularly mentioned above, the compositions may include other agents conventional in the art having regard to the type of formulation in question, for example, those suitable for oral administration may include flavoring agents.

### 4.4. Uses of the Pharmaceutical Compositions and Compounds in Therapy

A therapeutically effective amount of the agent will depend upon a number of factors including, for example, the age and weight of the subject, the precise condition requiring treatment and its severity, the nature of the formulation, and the route of administration, and will ultimately be at the discretion of the attendant physician or veterinarian. In particular, the subject to be treated is a mammal, particularly a human.

The agent may be administered in a daily dose. This amount may be given in a single dose per day or more usually in a number (e.g., two, three, four, five, or six) of sub-doses per day such that the total daily dose is the same.

Suitably, the amount of the compound of the present disclosure administered may be an amount selected from 0.01 mg to 10 g per day (calculated as the free or unsalted compound).

In some embodiments, a provided compound or a pharmaceutically acceptable salt thereof may be employed alone or in combination with other therapeutic agents. A provided compound or a pharmaceutically acceptable salt thereof and the other pharmaceutically active agent(s) may be administered together or separately and, when administered separately, administration may occur simultaneously or sequentially, in any order, by any convenient route in separate or combined pharmaceutical compositions. The amounts of a provided compound or a pharmaceutically acceptable salt thereof and the other pharmaceutically active agent(s) and the relative timings of administration will be selected in order to achieve the desired combined therapeutic effect. A provided compound or a pharmaceutically acceptable salt thereof and further therapeutic agent(s) may be employed in combination by administration simultaneously in a unitary pharmaceutical composition including both compounds. Alternatively, the combination may be administered separately in separate pharmaceutical compositions, each including one of the compounds in a sequential manner wherein, for example, the compound of the present disclosure is administered first and the other second and vice versa. Such sequential administration may be close in time (e.g. simultaneously) or remote in time. Furthermore, it does not matter if the compounds are administered in the same dosage form, e.g., one compound may be administered topically and the other compound may be administered orally. Suitably, both compounds are administered orally.

The combinations may be presented as a combination kit. By the term “combination kit” “or kit of parts” as used herein is meant the pharmaceutical composition or compositions that are used to administer the combination according to the present disclosure. When both compounds are administered simultaneously, the combination kit can contain both compounds in a single pharmaceutical composition, such as a tablet, or in separate pharmaceutical compositions. When the compounds are not administered simultaneously, the combination kit will contain each compound in separate pharmaceutical compositions either in a single package or in separate pharmaceutical compositions in separate packages. The combination kit can also be provided by instruction, such as dosage and administration instructions. Such dosage and administration instructions can be of the kind that are provided to a doctor, for example by a drug product label, or they can be of the kind that are provided by a doctor, such as instructions to a patient.

When the combination is administered separately in a sequential manner wherein one is administered first and the other second or vice versa, such sequential administration may be close in time or remote in time. For example, administration of the other agent several minutes to several dozen minutes after the administration of the first agent, and administration of the other agent several hours to several days after the administration of the first agent are included, wherein the lapse of time is not limited. For example, one agent may be administered once a day, and the other agent may be administered 2 or 3 times a day, or one agent may be administered once a week, and the other agent may be administered once a day and the like. It will be clear to a person skilled in the art that, where appropriate, the other therapeutic ingredients(s) may be used in the form of salts, for example as alkali metal or amine salts or as acid addition salts, or prodrugs, or as esters, for example lower alkyl esters, or as solvates, for example hydrates, to optimize the activity and/or stability and/or physical characteristics, such as solubility, of the therapeutic ingredient. It will be clear also that, where appropriate, the therapeutic ingredients may be used in optically pure form.

When combined in the same composition it will be appreciated that the two compounds must be stable and compatible with each other and the other components of the composition and may be formulated for administration. When formulated separately they may be provided in any convenient composition, conveniently, in such a manner as known for such compounds in the art.

When a provided compound or a pharmaceutically acceptable salt thereof is used in combination with a second therapeutic agent active against the same disease, condition, or disorder, the dose of each compound may differ from that when the compound is used alone. Appropriate doses will be readily appreciated by those skilled in the art.

In one embodiment, the mammal in the methods and uses of the present disclosure is a human. The provided compounds or pharmaceutically acceptable salts thereof are useful in the treatment of diseases and conditions in which modulation of cGAS is beneficial. As modulators of the immune response, a provided compound or a pharmaceutically acceptable salts thereof may also be useful, as stand-alone, in combination or as adjuvants, in the treatment of diseases and conditions in which modulation of cGAS is beneficial.

In one embodiment, the disease or condition is an inflammatory, allergic, or autoimmune diseases such as systemic lupus erythematosus, psoriasis, insulin-dependent diabetes mellitus (IDDM), scleroderma, Aicardi Goutieres syndrome, dermatomyositis, inflammatory bowel diseases, multiple sclerosis, rheumatoid arthritis, and Sjogren's syndrome (SS).

In another embodiment, the disease or condition is an infectious disease such as bacterial, viral or parasitic disease in which modulation of cGAS activity is beneficial.

In another embodiment, the disease or condition is a senescence- or age-related disease, including a neurodegenerative disease such as Alzheimer's or Parkinson disease, cardiovascular diseases such as atherosclerosis or myocardial infarction, liver or renal diseases, cancer, or premature aging.

Inflammation represents a group of vascular, cellular, and neurological responses to trauma. Inflammation can be characterized as the movement of inflammatory cells such as monocytes, neutrophils, and granulocytes into the tissues. This is usually associated with reduced endothelial barrier function and edema into the tissues. Inflammation can be classified as either acute or chronic. Acute inflammation is the initial response of the body to harmful stimuli and is achieved by the increased movement of plasma and leukocytes from the blood into the injured tissues. A cascade of biochemical event propagates and matures the inflammatory response, involving the local vascular system, the immune system, and various cells within the injured tissue. Prolonged inflammation, known as chronic inflammation, leads to a progressive shift in the type of cells which are present at the site of inflammation and is characterized by simultaneous destruction and healing of the tissue from the inflammatory process.

When occurring as part of an immune response to infection or as an acute response to trauma, inflammation can be beneficial and is normally self-limiting. However, inflammation can be detrimental under various conditions. This includes the production of excessive inflammation in response to infectious agents, which can lead to significant organ damage and death (e.g., in the setting of sepsis). Moreover, chronic inflammation is generally deleterious and is at the root of numerous chronic diseases, causing severe and irreversible damage to tissues. In such settings, the immune response is often directed against self-tissues (autoimmunity), although chronic responses to foreign entities can also lead to bystander damage to self-tissues. The aim of anti-inflammatory therapy is therefore to reduce this inflammation, to inhibit autoimmunity when present and to allow for the physiological process or healing and tissue repair to progress.

In some embodiments, a provided compound or a pharmaceutically acceptable salt thereof may be used to treat inflammation of any tissue and organs of the body, including musculoskeletal inflammation, vascular inflammation, neural inflammation, digestive system inflammation, ocular inflammation, inflammation of the reproductive system, and other inflammation, as exemplified below.

Musculoskeletal inflammation refers to any inflammatory condition of the musculoskeletal system, particularly those conditions affecting skeletal joints, including joints of the hand, wrist, elbow, shoulder, jaw, spine, neck, hip, knew, ankle, and foot, and conditions affecting tissues connecting muscles to bones such as tendons. Examples of musculoskeletal inflammation which may be treated with compounds of the present disclosure include arthritis (including, for example, osteoarthritis, rheumatoid arthritis, psoriatic arthritis, ankylosing spondylitis, acute and chronic infectious arthritis, arthritis associated with gout and pseudogout, and juvenile idiopathic arthritis), tendonitis, synovitis, tenosynovitis, bursitis, fibrositis (fibromyalgia), epicondylitis, myositis, and osteitis (including, for example, Paget's disease, osteitis pubis, and osteitis fibrosa cystic). Ocular inflammation refers to inflammation of any structure of the eye, including the eye lids. Examples of ocular inflammation which may be treated with the compounds of the present disclosure include blepharitis, blepharochalasis, conjunctivitis, dacryoadenitis, keratitis, keratoconjunctivitis sicca (dry eye), scleritis, trichiasis, and uveitis. Examples of inflammation of the nervous system which may be treated with the compounds of the present disclosure include encephalitis, Guillain-Barre syndrome, meningitis, neuromyotonia, narcolepsy, multiple sclerosis, myelitis, and schizophrenia.

Examples of inflammation of the vasculature or lymphatic system which may be treated with a provided compound or a pharmaceutically acceptable salt thereof include arthrosclerosis, arthritis, phlebitis, vasculitis, and lymphangitis.

Examples of inflammatory conditions of the digestive system which may be treated with a provided compound or a pharmaceutically acceptable salt thereof include cholangitis, cholecystitis, enteritis, enterocolitis, gastritis, gastroenteritis, inflammatory bowel disease (such as Crohn's disease and ulcerative colitis), ileitis, and proctitis.

Examples of inflammatory conditions of the reproductive system which may be treated with a provided compound or a pharmaceutically acceptable salt thereof include cervicitis, chorioamnionitis, endometritis, epididymitis, omphalitis, oophoritis, orchitis, salpingitis, tubo-ovarian abscess, urethritis, vaginitis, vulvitis, and vulvodynia.

The agents may be used to treat autoimmune conditions having an inflammatory component. Such conditions include systemic lupus erythematosus, acute disseminated alopecia universalis, Behcet's disease, Chagas' disease, chronic fatigue syndrome, dysautonomia, encephalomyelitis, ankylosing spondylitis, aplastic anemia, hidradenitis suppurativa, autoimmune hepatitis, autoimmune oophoritis, celiac disease, Crohn's disease, diabetes mellitus type 1, giant cell arteritis, goodpasture's syndrome. Grave's disease, Guillain-Barre syndrome, Hashimoto's disease, Henoch-Schonlein purpura, Kawasaki's disease, microscopic colitis, microscopic polyarteritis, mixed connective tissue disease, multiple sclerosis, myasthenia gravis, opsoclonus myoclonus syndrome, optic neuritis, ord's thyroiditis, pemphigus, polyarteritis nodosa, polymyalgia, rheumatoid arthritis, Reiter's syndrome, Sjogren's syndrome, Aicardi Goutieres syndrome, temporal arteritis, Wegener's granulomatosis, warm autoimmune haemolytic anemia, interstitial cystitis, Lyme disease, morphea, psoriasis, sarcoidosis, scleroderma, ulcerative colitis, and vitiligo.

In some embodiments, a provided compound or a pharmaceutically acceptable salt thereof may be used to treat T-cell mediated hypersensitivity diseases having an inflammatory component. Such conditions include contact hypersensitivity, contact dermatitis (including that due to poison ivy), uticaria, skin allergies, respiratory allergies (hayfever, allergic rhinitis) and gluten-sensitive enteropathy (Celiac disease).

Other inflammatory conditions which may be treated with a provided compound or a pharmaceutically acceptable salt thereof include, for example, appendicitis, dermatitis, dermatomyositis, endocarditis, fibrositis, gingivitis, glossitis, hepatitis, hidradenitis suppurativa, iritis, laryngitis, mastitis, myocarditis, nephritis, otitis, pancreatitis, parotitis, pericarditis, peritonitis, pharyngitis, pleuritis, pneumonitis, prostatitis, pyelonephritis, and stomatitis, transplant rejection (involving organs such as kidney, liver, heart, lung, pancreas (e.g., islet cells), bone marrow, cornea, small bowel, skin allografts, skin homografts, and heart valve xenografts, serum sickness, and graft vs host disease), acute pancreatitis, chronic pancreatitis, acute respiratory distress syndrome, Sezary's syndrome, congenital adrenal hyperplasia, nonsuppurative thyroiditis, hypercalcemia associated with cancer, pemphigus, bullous dermatitis herpetiformis, severe erythema multiforme, exfoliative dermatitis, seborrheic dermatitis, seasonal or perennial allergic rhinitis, bronchial asthma, contact dermatitis, atopic dermatitis, drug hypersensitivity reactions, allergic conjunctivitis, keratitis, herpes zoster ophthalmicus, iritis and iridocyclitis, chorioretinitis, optic neuritis, symptomatic sarcoidosis, fulminating or disseminated pulmonary tuberculosis chemotherapy, idiopathic thrombocytopenic purpura in adults, secondary thrombocytopenia in adults, acquired (autoimmune) haemolytic anemia, leukemia and lymphomas in adults, acute leukemia of childhood, regional enteritis, autoimmune vasculitis, multiple sclerosis, chronic obstructive pulmonary disease, solid organ transplant rejection, sepsis. Preferred treatments include treatment of transplant rejection, rheumatoid arthritis, psoriatic arthritis, multiple sclerosis. Type 1 diabetes, asthma, inflammatory bowel disease, systemic lupus erythematosus, psoriasis, chronic pulmonary disease, and inflammation accompanying infectious conditions (e.g., sepsis).

In some embodiments, the disclosure provides a provided compound or a pharmaceutically acceptable salt thereof for use in the treatment of an inflammatory, allergic, or autoimmune disease.

In some embodiments, the disclosure provides a method of treating an inflammatory, allergic, or autoimmune disease comprising: administering to a patient in need thereof a therapeutically effective amount of a provided compound or a pharmaceutically acceptable salt thereof.

In some embodiments, the disclosure provides the use of a provided compound or a pharmaceutically acceptable salt thereof in the manufacture of a medicament for the treatment of an inflammatory, allergic, or autoimmune disease.

In some embodiments, a provided compound or a pharmaceutically acceptable salts thereof may be used in combination with one or more other agents in the prevention or treatment of an allergic inflammatory autoimmune disease, wherein such other agents can include: antigen immunotherapy agents; antihistamines; steroids, NSAIDs; bronchodilators (e.g. beta 2 agonists, adrenergic agonists, anticholinergic agents, theophylline); methotrexate; leukotriene modulators; monoclonal antibody agents such as anti-lgE, anti-TNF, anti-IL-5, anti-IL-6, anti-IL-12, anti-IL-1 and similar agents; receptor therapies agents such as etanercept; and antigen non-specific immunotherapeutic agents such interferon or other cytokines/chemokines, cytokine/chemokine receptor modulators, cytokine agonists or antagonists, and TLR antagonist.

In some embodiments, the present disclosure provides a pharmaceutical composition comprising a provided compound or a pharmaceutically acceptable salt thereof, and at least one further therapeutic agent useful in the treatment of an allergic, inflammatory, or autoimmune disease.

In some embodiments, the present disclosure provides a pharmaceutical composition comprising a provided compound or a pharmaceutically acceptable salt thereof, and at least one further therapeutic agent useful in the treatment of an allergic, inflammatory, or autoimmune disease for use in therapy.

In some embodiments, the present disclosure provides a pharmaceutical composition comprising a provided compound or pharmaceutically acceptable salt thereof, and at least one further therapeutic agent useful in the treatment of an allergic inflammatory or autoimmune disease, for use in the treatment of allergic, inflammatory, or autoimmune disease.

In some embodiments, the present disclosure provides the use of a pharmaceutical composition comprising a provided compound or a pharmaceutically acceptable salt thereof, and at least one further therapeutic agent useful in the treatment of an allergic, inflammatory, or autoimmune disease in the manufacture of a medicament for the treatment of an allergic, inflammatory or autoimmune disease.

In some embodiments, the present disclosure provides a method of treating an allergic, inflammatory or autoimmune disease comprising: administering to a patient in need thereof a therapeutically effective amount of a pharmaceutical composition comprising a provided compound or a pharmaceutically acceptable salt thereof, and at least one further therapeutic agent useful in the treatment of an allergic, inflammatory, or autoimmune disease.

In some embodiments, the present disclosure provides a pharmaceutical composition comprising a provided compound or a pharmaceutically acceptable salt thereof, at least one further therapeutic agent useful in the treatment of an allergic, inflammatory, or autoimmune disease, and one or more of pharmaceutically acceptable excipients.

In some embodiments, the present disclosure provides a provided compound or a pharmaceutically acceptable salt thereof, for use in the treatment of an infectious disease.

In some embodiments, the present disclosure provides a method of treating an infectious disease comprising administering to a patient in need thereof a therapeutically effective amount of a provided compound or a pharmaceutically acceptable salt thereof.

In some embodiments, the present disclosure provides the use of a provided compound or a pharmaceutically acceptable salt thereof, in the manufacture of a medicament for the treatment of an infectious disease. In one embodiment, a compound of the present disclosure may be employed with other therapeutic methods of treating infectious disease. In particular, bacterial and parasite infections, such as Mycobacterium tuberculosis and malaria, respectively, which exploit the type-I interferon pathway for their advantage, may be treated with a cGAS inhibitor.

In some embodiments, a provided compound or a pharmaceutically acceptable salts thereof may be used in combination with one or more agents useful in the prevention or treatment of bacterial and viral infections. Examples of such agents include: polymerase inhibitors; replication inhibitors such as acyclovir, famciclovir, ganciclovir, cidofovir and lamivudine; protease inhibitors such as the HIV protease inhibitors saquinavir, ritonavir, indinavir, nelfinavir, amprenavir, fosamprenavir, brecanavir, atazanavir, tipranavir, palinavir, lasinavir, and the HCV protease inhibitors BILN2061, VX-950, SCH503034; nucleoside and nucleotide reverse transcriptase inhibitors such as zidovudine, didanosine, lamivudine, zalcitabine, abacavir, stavudine, adefovir, adefovir dipivoxil, fozivudine, todoxil, emtricitabine, alovudine, amdoxovir, and elvucitabine; non-nucleoside reverse transcriptase inhibitors (including an agent having antioxidation activity such as immunocal or oltipraz) such as nevirapine, delavirdine, efavirenz, loviride, immunocal, oltipraz, capravirine, TMC-278, TMC-125, and etravirine; entry inhibitors such as enfuvirtide (T-20), T-1249, PRO-542, PRO-140, TNX-355, BMS-806, 5-Helix and similar agents; integrase inhibitors such as L-870 and 180; budding inhibitors such as PA-344 and PA-457; chemokine receptor inhibitors such as vicriviroc (Sch-C), Sch-D, TAK779, maraviroc (UK-427,857), and TAK449; neuraminidase inhibitors such as CS-8958, zanamivir, oseltamivir, and peramivir; ion channel blockers such as amantadine or rimantadine; interfering RNA and antisense oligonucleotides and such as ISIS-14803; and antiviral agents of undetermined mechanism of action, such as ribavirin.

In some embodiments, a provided compound or a pharmaceutically acceptable salts thereof may also be used in combination with one or more other agents which may be useful in the prevention or treatment of viral infections such as immune therapies (e.g., interferon or other cytokines/chemokines, cytokine/chemokine receptor modulators, cytokine agonists or antagonists and similar agents); therapeutic vaccines; antifibrotic agents; and anti-inflammatory agents such as corticosteroids or NSAIDs (non-steroidal anti-inflammatory agents).

In some embodiments, the present disclosure provides a pharmaceutical composition comprising a provided compound or a pharmaceutically acceptable salt thereof, and at least one further therapeutic agent useful in the treatment of an infectious disease.

In some embodiments, the present disclosure provides a pharmaceutical composition comprising a provided compound or a pharmaceutically acceptable salt thereof, and at least one further therapeutic agent useful in the treatment of an infectious disease for use in therapy.

In some embodiments, the present disclosure provides a pharmaceutical composition comprising a provided compound or pharmaceutically acceptable salt thereof, and at least one further therapeutic agent useful in the treatment of an infectious disease, for use in the treatment of an infectious disease.

In some embodiments, the present disclosure provides the use of a pharmaceutical composition comprising a provided compound or a pharmaceutically acceptable salt thereof, and at least one further therapeutic agent useful in the treatment of an infectious disease in the manufacture of a medicament for the treatment of an infectious disease.

In some embodiments, the present disclosure provides a method of treating an infectious disease comprising administering to a patient in need thereof, a therapeutically effective amount of a pharmaceutical composition comprising a provided compound or a pharmaceutically acceptable salt thereof, and at least one further therapeutic agent useful in the treatment of an infectious disease.

In some embodiments, the present disclosure provides a pharmaceutical composition comprising a provided compound or a pharmaceutically acceptable salt thereof, at least one further therapeutic agent useful in the treatment of infectious disease, and one or more of pharmaceutically acceptable excipients.

In some embodiments, the disclosure provides a contemplated compound herein or a pharmaceutically acceptable salt thereof for use in the treatment of a senescence- or age-related disease.

In some embodiments, the disclosure provides a method of treating a senescence- or age-related disease comprising: administering to a patient in need thereof a therapeutically effective amount of a provided compound or a pharmaceutically acceptable salt thereof.

In some embodiments, the disclosure provides the use of a contemplated compound herein or a pharmaceutically acceptable salt thereof in the manufacture of a medicament for the treatment of a senescence- or age-related disease.

In some embodiments, the present disclosure provides a pharmaceutical composition comprising a contemplated compound herein or a pharmaceutically acceptable salt thereof, and at least one further therapeutic agent useful in the treatment of a senescence- or age-related disease.

In some embodiments, the present disclosure provides a pharmaceutical composition comprising a contemplated compound herein or a pharmaceutically acceptable salt thereof, and at least one further therapeutic agent useful in the treatment of a senescence- or age-related disease for use in therapy.

In some embodiments, the present disclosure provides a pharmaceutical composition comprising a contemplated compound herein or pharmaceutically acceptable salt thereof, and at least one further therapeutic agent useful in the treatment of a senescence- or age-related disease, for use in the treatment of a senescence- or age-related disease.

In some embodiments, the present disclosure provides the use of a pharmaceutical composition comprising a contemplated compound herein or a pharmaceutically acceptable salt thereof, and at least one further therapeutic agent useful in the treatment of a senescence- or age-related disease in the manufacture of a medicament for the treatment of a senescence- or age-related disease.

In some embodiments, the present disclosure provides a method of treating a senescence- or age-related disease comprising administering to a patient in need thereof, a therapeutically effective amount of a pharmaceutical composition comprising a contemplated compound herein or a pharmaceutically acceptable salt thereof, and at least one further therapeutic agent useful in the treatment of a senescence- or age-related disease.

In some embodiments, the present disclosure provides a pharmaceutical composition comprising a contemplated compound herein or a pharmaceutically acceptable salt thereof, at least one further therapeutic agent useful in the treatment of a senescence- or age-related disease, and one or more of pharmaceutically acceptable excipients.

The contemplated compounds herein may be prepared by methods known in the art of organic synthesis as set forth in the schemes below and/or the specific Examples described below. In all of the methods, it is well understood that protecting groups for sensitive or reactive groups may be employed where necessary in accordance with general principles of chemistry. Protecting groups are manipulated according to standard methods of organic synthesis (T. W. Green and P. G. M. Wuts (1999) Protective Groups in Organic Synthesis, 3rd edition, John Wiley & Sons). These groups are removed at a convenient stage of the compound synthesis using methods that are readily apparent to those skilled in the art. The selection of processes as well as the reaction conditions and order of their execution shall be consistent with the preparation of the provided compounds.

The following list provides definitions of certain abbreviations as used herein. It will be appreciated that the list is not exhaustive, but the meaning of those abbreviations not herein below defined will be readily apparent to those skilled in the art: AIBN is 2,2′-azobisisobutyronitrile; ATP is adenosine 5′-triphosphate; BPO is benzoyl peroxide; n-BuLi is n-butyllithium; BzCI is benzoyl chloride; CDI is 1,1′-carbonyldiimidazole; cGAS is cyclic GMP-AMP synthase; CO is carbon monoxide; Cu(OAc)₂ is copper(II) acetate; CuCN is copper(I) cyanide; CuI is copper(I) iodide; DAST is (diethylamino)sulfur trifluoride; DBU is 1,8-diazabicyclo[5.4.0]undec-7-ene; DCE is dichloroethane; DCM is dichloromethane; DDQ is 2,3-dichloro-5,6-dicyano-p-benzoquinone; DHP is 3,4-dihydro-2H-pyran; DIAD is diisopropyl azodicarboxylate; DIBAL-H is diisobutylaluminum hydride; DIPA is diisopropylamine; DIPEA is N,N-diisopropylethylamine; DMAP is 4-(dimethylamino)pyridine; DMB is 2,4-dimethoxybenzyl; DMF is N,N-dimethylformamide; DMP is Dess-Martin periodinane; DMSO is dimethyl sulfoxide; EA is ethyl acetate; EtMgBr is ethylmagnesium bromide; Et₂O is diethyl ether; EtOH is ethanol; GTP is guanosine triphosphate; HCl is hydrochloric acid; HMTA is hexamethylenetetramine; HOAc is acetic acid; HPLC is high performance liquid chromatography; LAH is lithium aluminum hydride; mCPBA is 3-chloroperbenzoic acid; MeCN is acetonitrile; Mel is iodomethane; MEK is methyl ethyl ketone or butanone; MeOH is methanol; MeMgBr is methylmagnesium bromide; MOMCl is chloromethyl methyl ether; MOM is methoxymethoxy; MS is mass spectrometer or mass spectrum; MsCl is methanesulfonyl chloride; MTBE is methyl tert-butyl ether; NaH is sodium hydride; NaOH is sodium hydroxide; NBS is N-bromosuccinimide; NMM is N-methylmorpholine; NMR is nuclear magnetic resonance; Pd(dba)₂ is bis(dibenzylideneacetone)palladium(O); Pd(dppf)Cl₂ is [1,1′-bis(diphenylphosphino)ferrocene]dichloropalladium(II); Pd(OAc)₂ is palladium(II) acetate; Pd(PPh₃)₂Cl₂ is bis(triphenylphosphine)palladium(II) dichloride; Pd(PPh₃)₄ is tetrakis(triphenylphosphine) palladium(O); Pd/C is palladium on carbon; PDC is pyridinium dichromate; PE is petroleum ether; PMB is 4-methoxybenzyl; PPh₃ is triphenylphosphine; prep-HPLC is preparative high performance liquid chromatography; prep-TLC is preparative thin-layer chromatography; Py is pyridine; TBAF is tetra-n-butylammonium fluoride; TBSCl is tert-butyldimethylsilyl chloride; TEA is triethylamine; TFA is trifluoroacetic acid; THF is tetrahydrofuran; THP is tetrahydropyranyl; TLC is thin-layer chromatography; TSA is p-toluenesulfonic acid monohydrate, and TsC1 is p-toluenesulfonyl chloride.

### 4.5. Embodiments

In one aspect, embodiments herein relate to solid forms of Compound A:

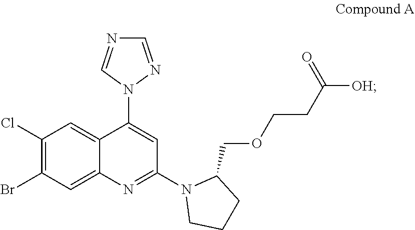 [SMILES: O=C(O)CCOC[C@@H]1CCCN1c1cc(-n2cncn2)c2cc(Cl)c(Br)cc2n1]

In some embodiments, the solid form of Compound A is freeform Type B.

In some embodiments, the solid form of Compound A exhibits a differential scanning calorimetry thermogram having an endotherm onset value at 144.3±2° C.

In some embodiments, Compound A freeform Type B has an X-ray diffraction pattern with four characteristic peaks expressed in values of degrees 2θ at 6.3±0.2, 9.9±0.2, 16.0±0.2, and 22.7±0.2. In some embodiments, Compound A freeform Type B has an X-ray diffraction pattern with five characteristic peaks expressed in values of degrees 2θ at 6.3±0.2, 9.9±0.2, 16.0±0.2, 18.3±0.2, and 22.7±0.2. In some embodiments, Compound A freeform Type B has an X-ray diffraction pattern with six characteristic peaks expressed in values of degrees 2θ at 6.3±0.2, 9.9±0.2, 16.0±0.2, 18.3±0.2, 22.7±0.2, and 25.6±0.2. In some embodiments, Compound A freeform Type B has an X-ray diffraction pattern with seven characteristic peaks expressed in values of degrees 2θ such as seven characteristic peaks at 6.3±0.2, 9.9±0.2, 12.7±0.2, 16.1±0.2, 18.3±0.2, 22.7±0.2, and 25.6±0.2. In some embodiments, Compound A freeform Type B has an X-ray diffraction pattern with eight characteristic peaks expressed in values of degrees 2θ at 6.3±0.2, 9.9±0.2, 12.7±0.2, 16.1±0.2, 17.7±0.2, 18.3±0.2, 22.7±0.2, and 25.6±0.2. In some embodiments, Compound A freeform Type B has an X-ray diffraction pattern with nine characteristic peaks expressed in values of degrees 2θ at 6.3±0.2, 9.9±0.2, 12.7±0.2, 16.1±0.2, 17.7±0.2, 18.3±0.2, 22.7±0.2, 25.6±0.2, and 26.2±0.2. In some embodiments, Compound A freeform Type B has an X-ray diffraction pattern with ten characteristic peaks expressed in values of degrees 2θ at 6.3±0.2, 9.9±0.2, 12.7±0.2, 16.1±0.2, 17.7±0.2, 18.3±0.2, 20.4±0.2, 22.7±0.2, 25.6±0.2, and 26.2±0.2. In some embodiments, Compound A freeform Type B has an X-ray diffraction pattern with eleven characteristic peaks expressed in values of degrees 2θ at 6.3±0.2, 9.9±0.2, 12.7±0.2, 16.1±0.2, 17.7±0.2, 18.3±0.2, 20.4±0.2, 22.7±0.2, 24.0±0.2, 25.6±0.2, and 26.2±0.2. In some embodiments, Compound A freeform Type B has an X-ray diffraction pattern with twelve characteristic peaks expressed in values of degrees 2θ at 6.3±0.2, 9.9±0.2, 12.7±0.2, 16.1±0.2, 17.7±0.2, 18.3±0.2, 19.9±0.2, 20.4±0.2, 22.7±0.2, 24.0±0.2, 25.6±0.2, and 26.2±0.2. In some embodiments, Compound A freeform Type B has an X-ray diffraction pattern with thirteen characteristic peaks expressed in values of degrees 2θ at 6.3±0.2, 9.9±0.2, 12.7±0.2, 16.1±0.2, 17.7±0.2, 18.3±0.2, 19.1±0.2, 19.9±0.2, 20.4±0.2, 22.7±0.2, 24.0±0.2, 25.6±0.2, and 26.2±0.2.

In some embodiments, the solid form of Compound A has an X-ray diffraction pattern with at least three (such as at least four, five, six, seven, eight, nine, ten, eleven, twelve, or thirteen, fourteen, fifteen, sixteen, seventeen, eighteen, or nineteen) characteristic peaks expressed in values of degrees 2θ selected from those at 6.3±0.2, 8.8±0.2, 9.9±0.2, 12.7±0.2, 16.1±0.2, 17.7±0.2, 18.3±0.2, 19.1±0.2, 19.9±0.2, 20.4±0.2, 22.7±0.2, 23.1±0.2, 24.0±0.2, 25.6±0.2, 26.2±0.2, 26.6±0.2, 28.3±0.2, 31.6±0.2, and 32.6±0.2.

In some embodiments, the solid form of Compound A is freeform Type A.

In some embodiments, Compound A freeform Type A has an X-ray diffraction pattern with characteristic peaks expressed in values of degrees 2θ with at least three (such as at least four, five, six, seven, eight, or nine) characteristic peaks expressed in values of degrees 2θ selected from those at 7.4±0.2, 8.8±0.2, 10.6±0.2, 16.2±0.2, 18.5±0.2, 22.0±0.2, 22.9±0.2, 25.0±0.2, and 26.4±0.2.

In another aspect, embodiments herein relate to a pharmaceutically acceptable salt of Compound A:

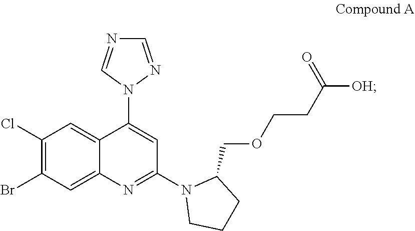 [SMILES: O=C(O)CCOC[C@@H]1CCCN1c1cc(-n2cncn2)c2cc(Cl)c(Br)cc2n1]

wherein the pharmaceutically acceptable salt is selected from hydrochloride salt, sulfate, napadisylate, esylate, mesylate, napsylate, besylate, sodium salt, potassium salt, L-arginine salt, choline salt, L-lysine salt, t-butylamine salt, meglumine salt, and tris(hydroxymethyl)aminomethane salt.

In some embodiments, the pharmaceutically acceptable salt of Compound A is a solid form.

In some embodiments, the pharmaceutically acceptable salt of Compound A is amorphous.

In some embodiments, the pharmaceutically acceptable salt of Compound A is crystalline.

In some embodiments, the pharmaceutically acceptable salt of Compound A is a non-solid, In further embodiments, the pharmaceutically acceptable salt of Compound A is a gel. In still further embodiments, the pharmaceutically acceptable salt of Compound A is an oil.

In some embodiments, the pharmaceutically acceptable salt of Compound A is selected from esylate, t-butylamine salt, and tris(hydroxymethyl)aminomethane salt.

In some embodiments, the pharmaceutically acceptable salt of Compound A is esylate. In a further embodiment, the Compound A esylate is in a solid form.

In some embodiments, the solid form of Compound A esylate has an X-ray diffraction pattern with four characteristic peaks expressed in values of degrees 2θ at 6.0±0.2, 18.6±0.2, 19.5±0.2, and 20.9±0.2. In some embodiments, the solid form of Compound A esylate has an X-ray diffraction pattern with five characteristic peaks expressed in values of degrees 2θ at 6.0±0.2, 18.6±0.2, 19.5±0.2, 20.9±0.2, and 25.4±0.2. In some embodiments, the solid form of Compound A esylate has an X-ray diffraction pattern with six characteristic peaks expressed in values of degrees 2θ at 6.0±0.2, 18.6±0.2, 19.5±0.2, 20.9±0.2, 22.1±0.2, and 25.4±0.2. In some embodiments, the solid form of Compound A esylate has an X-ray diffraction pattern with seven characteristic peaks expressed in values of degrees 2θ at 6.0±0.2, 11.5±0.2, 18.6±0.2, 19.5±0.2, 20.9±0.2, 22.1±0.2, and 25.4±0.2. In some embodiments, the solid form of Compound A esylate has an X-ray diffraction pattern with eight characteristic peaks expressed in values of degrees 2θ at 6.0±0.2, 11.5±0.2, 18.6±0.2, 19.5±0.2, 20.9±0.2, 22.1±0.2, 22.8±0.2, and 25.4±0.2. In some embodiments, the solid form of Compound A esylate has an X-ray diffraction pattern with nine characteristic peaks expressed in values of degrees 2θ at 6.0±0.2, 11.5±0.2, 13.3±0.2, 18.6±0.2, 19.5±0.2, 20.9±0.2, 22.1±0.2, 22.8±0.2, and 25.4±0.2. In some embodiments, the solid form of Compound A esylate has an X-ray diffraction pattern with ten characteristic peaks expressed in values of degrees 2θ at 6.0±0.2, 11.5±0.2, 13.3±0.2, 18.6±0.2, 19.5±0.2, 20.9±0.2, 22.1±0.2, 22.8±0.2, 24.6±0.2, and 25.4±0.2. In some embodiments, the solid form of Compound A esylate has an X-ray diffraction pattern with eleven characteristic peaks expressed in values of degrees at 6.0±0.2, 11.5±0.2, 13.3±0.2, 18.6±0.2, 19.5±0.2, 20.9±0.2, 22.1±0.2, 22.8±0.2, 23.9±0.2, 24.6±0.2, and 25.4±0.2. In some embodiments, the solid form of Compound A esylate has an X-ray diffraction pattern with twelve characteristic peaks expressed in values of degrees 2θ at 6.0±0.2, 11.5±0.2, 13.3±0.2, 18.6±0.2, 19.5±0.2, 20.9±0.2, 22.1±0.2, 22.8±0.2, 23.9±0.2, 24.6±0.2, 25.4±0.2, and 29.1±0.2.

In some embodiments, the solid form of Compound A esylate has an X-ray diffraction pattern with at least three (such as at least four, five, six, seven, eight, nine, ten, eleven, or twelve) characteristic peaks expressed in values of degrees 2θ selected from those at 6.0±0.2, 11.5±0.2, 13.3±0.2, 18.6±0.2, 19.5±0.2, 20.9±0.2, 22.1±0.2, 22.8±0.2, 23.9±0.2, 24.6±0.2, 25.4±0.2, and 29.1±0.2.

In some embodiments, the solid form of Compound A esylate exhibits a differential scanning calorimetry (DSC) thermogram having an endotherm onset value at 211.8±2° C.

In some embodiments, the solid form of Compound A esylate exhibits a differential scanning calorimetry (DSC) thermogram having an endotherm onset value at 208.0±2° C.

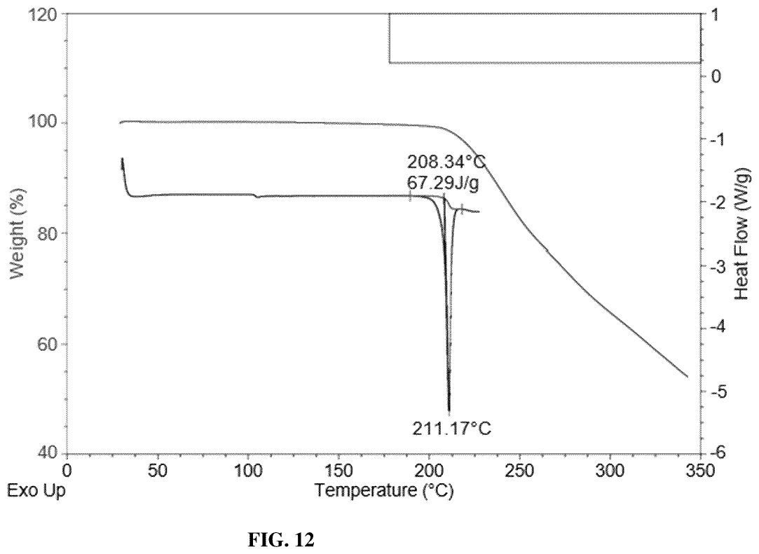

In some embodiments, the solid form of Compound A esylate exhibits a thermogravimetric analysis thermogram/differential scanning calorimetry thermogram pattern substantially similar to that of FIG. 12.

In some embodiments, the pharmaceutically acceptable salt of Compound A is t-butylamine salt. In a further embodiment, the pharmaceutically acceptable salt of Compound A is t-butylamine salt is in a solid form.

In some embodiments, the solid form of Compound A t-butylamine salt has an X-ray diffraction pattern with four characteristic peaks expressed in values of degrees 2θ at 6.4±0.2, 11.4±0.2, 13.4±0.2, and 21.0±0.2. In some embodiments, the solid form of Compound A t-butylamine salt has an X-ray diffraction pattern with five characteristic peaks expressed in values of degrees 2θ at 6.4±0.2, 6.6±0.2, 11.4±0.2, 13.4±0.2, and 21.0±0.2. In some embodiments, the solid form of Compound A t-butylamine salt has an X-ray diffraction pattern with six characteristic peaks expressed in values of degrees 2θ at 6.4±0.2, 6.6±0.2, 11.4±0.2, 13.4±0.2, 21.0±0.2, and 21.4±0.2. In some embodiments, the solid form of Compound A t-butylamine salt has an X-ray diffraction pattern with seven characteristic peaks expressed in values of degrees 2θ at 6.4±0.2, 6.6±0.2, 11.4±0.2, 13.4±0.2, 21.0±0.2, 21.4±0.2, and 26.1±0.2. In some embodiments, the solid form of Compound A t-butylamine salt has an X-ray diffraction pattern with eight characteristic peaks expressed in values of degrees 2θ at 6.4±0.2, 6.6±0.2, 11.4±0.2, 13.4±0.2, 17.9±0.2, 21.0±0.2, 21.4±0.2, and 26.1±0.2. In some embodiments, the solid form of Compound A t-butylamine salt has an X-ray diffraction pattern with nine characteristic peaks expressed in values of degrees 2θ at 6.4±0.2, 6.6±0.2, 11.4±0.2, 13.4±0.2, 15.0±0.2, 17.9±0.2, 21.0±0.2, 21.4±0.2, and 26.1±0.2. In some embodiments, the solid form of Compound A t-butylamine salt has an X-ray diffraction pattern with ten characteristic peaks expressed in values of degrees 2θ at 6.4±0.2, 6.6±0.2, 11.4±0.2, 13.4±0.2, 15.0±0.2, 17.9±0.2, 21.0±0.2, 21.4±0.2, 22.1±0.2, and 26.1±0.2. In some embodiments, the solid form of Compound A t-butylamine salt has an X-ray diffraction pattern with eleven characteristic peaks expressed in values of degrees 2θ at 6.4±0.2, 6.6±0.2, 11.4±0.2, 13.4±0.2, 15.0±0.2, 17.7±0.2, 17.9±0.2, 21.0±0.2, 21.4±0.2, 22.1±0.2, and 26.1±0.2.

In some embodiments, the solid form of Compound A t-butylamine salt has an X-ray diffraction pattern with at least three (such as four, five, six, seven, eight, nine, ten, or eleven) characteristic peaks expressed in values of degrees 2θ selected from those at 6.4±0.2, 6.6±0.2, 11.4±0.2, 13.4±0.2, 15.0±0.2, 17.7±0.2, 17.9±0.2, 21.0±0.2, 21.4±0.2, 22.1±0.2, and 26.1±0.2.

In some embodiments, the solid form of Compound A t-butylamine salt exhibits a differential scanning calorimetry thermogram having an endotherm onset value at 168.6±2° C.

In some embodiments, the solid form of Compound A t-butylamine salt exhibits a differential scanning calorimetry thermogram having an endotherm onset value at 166.7±2° C.

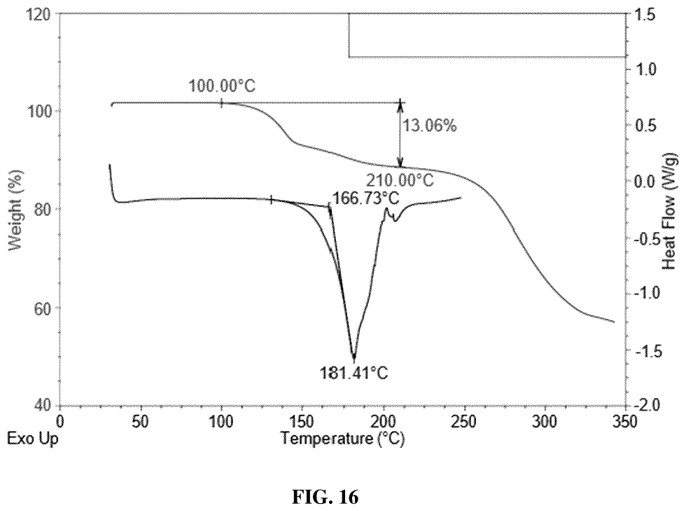

In some embodiments, the solid form of Compound A t-butylamine salt exhibits a thermogravimetric analysis thermogram/differential scanning calorimetry thermogram pattern substantially similar to that of FIG. 16.

In some embodiments, the pharmaceutically acceptable salt of Compound A is tris(hydroxymethyl)aminomethane salt. In further embodiments, the Compound A tris(hydroxymethyl)aminomethane salt is in a solid form.

In embodiments, the solid form of Compound A tris(hydroxymethyl)aminomethane salt has an X-ray diffraction pattern with five characteristic peaks expressed in values of degrees 2θ at 7.5±0.2, 18.8±0.2, 19.0±0.2, 22.4±0.2, and 22.6±0.2. In embodiments, the solid form of Compound A tris(hydroxymethyl)aminomethane salt has an X-ray diffraction pattern with six characteristic peaks expressed in values of degrees 2θ at 7.5±0.2, 18.8±0.2, 19.0±0.2, 21.5±0.2, 22.4±0.2, and 22.6±0.2. In embodiments, the solid form of Compound A tris(hydroxymethyl)aminomethane salt has an X-ray diffraction pattern with seven characteristic peaks expressed in values of degrees 2θ at 7.5±0.2, 18.8±0.2, 19.0±0.2, 21.5±0.2, 22.4±0.2, 22.6±0.2, and 27.8±0.2. In embodiments, the solid form of Compound A tris(hydroxymethyl)aminomethane salt has an X-ray diffraction pattern with eight characteristic peaks expressed in values of degrees 2θ at 7.5±0.2, 18.8±0.2, 19.0±0.2, 20.6±0.2, 21.5±0.2, 22.4±0.2, 22.6±0.2, and 27.8±0.2. In embodiments, the solid form of Compound A tris(hydroxymethyl)aminomethane salt has an X-ray diffraction pattern with nine characteristic peaks expressed in values of degrees 2θ at 7.5±0.2, 18.8±0.2, 19.0±0.2, 19.7±0.2, 20.6±0.2, 21.5±0.2, 22.4±0.2, 22.6±0.2, and 27.8±0.2. In embodiments, the solid form of Compound A tris(hydroxymethyl)aminomethane salt has an X-ray diffraction pattern with ten characteristic peaks expressed in values of degrees 2θ at 3.7±0.2, 7.5±0.2, 18.8±0.2, 19.0±0.2, 19.7±0.2, 20.6±0.2, 21.5±0.2, 22.4±0.2, 22.6±0.2, and 25.0±0.2. In embodiments, the solid form of Compound A tris(hydroxymethyl)aminomethane salt has an X-ray diffraction pattern with eleven characteristic peaks expressed in values of degrees 2θ at 3.7±0.2, 7.5±0.2, 18.8±0.2, 19.0±0.2, 19.7±0.2, 20.6±0.2, 21.5±0.2, 22.4±0.2, and 22.6±0.2, 25.0±0.2, 27.8±0.2. In embodiments, the solid form of Compound A tris(hydroxymethyl)aminomethane salt has an X-ray diffraction pattern with twelve characteristic peaks expressed in values of degrees 2θ at 3.7±0.2, 7.5±0.2, 18.8±0.2, 19.0±0.2, 19.7±0.2, 20.6±0.2, 21.5±0.2, 22.4±0.2, 22.6±0.2, 25.0±0.2, 26.4±0.2, and 27.8±0.2. In embodiments, the solid form of Compound A tris(hydroxymethyl)aminomethane salt has an X-ray diffraction pattern with thirteen characteristic peaks expressed in values of degrees 2θ at 3.7±0.2, 7.5±0.2, 18.8±0.2, 19.0±0.2, 19.7±0.2, 20.6±0.2, 21.5±0.2, 22.4±0.2, 22.6±0.2, 25.0±0.2, 25.7±0.2, 26.4±0.2, and 27.8±0.2. In embodiments, the solid form of Compound A tris(hydroxymethyl)aminomethane salt has an X-ray diffraction pattern with fourteen characteristic peaks expressed in values of degrees 2θ at 3.7±0.2, 7.5±0.2, 18.8±0.2, 19.0±0.2, 19.7±0.2, 20.6±0.2, 21.5±0.2, 22.4±0.2, 22.6±0.2, 25.0±0.2, 25.7±0.2, 26.4±0.2, 27.8±0.2, and 28.7±0.2. In embodiments, the solid form of Compound A tris(hydroxymethyl)aminomethane salt has an X-ray diffraction pattern with fifteen characteristic peaks expressed in values of degrees 2θ at 3.7±0.2, 7.5±0.2, 18.8±0.2, 19.0±0.2, 19.7±0.2, 20.6±0.2, 21.5±0.2, 22.4±0.2, 22.6±0.2, 25.0±0.2, 25.7±0.2, 26.4±0.2, 27.8±0.2, 28.7±0.2, and 29.3±0.2. In embodiments, the solid form of Compound A tris(hydroxymethyl)aminomethane salt has an X-ray diffraction pattern with sixteen characteristic peaks expressed in values of degrees 2θ at 3.7±0.2, 7.5±0.2, 17.8±0.2, 18.8±0.2, 19.0±0.2, 19.7±0.2, 20.6±0.2, 21.5±0.2, 22.4±0.2, 22.6±0.2, 25.0±0.2, 25.7±0.2, 26.4±0.2, 27.8±0.2, 28.7±0.2, and 29.3±0.2. In embodiments, the solid form of Compound A tris(hydroxymethyl)aminomethane salt has an X-ray diffraction pattern with seventeen characteristic peaks expressed in values of degrees 2θ at 3.7±0.2, 7.5±0.2, 14.9±0.2, 17.8±0.2, 18.8±0.2, 19.0±0.2, 19.7±0.2, 20.6±0.2, 21.5±0.2, 22.4±0.2, 22.6±0.2, 25.0±0.2, 25.7±0.2, 26.4±0.2, 27.8±0.2, 28.7±0.2, and 29.3±0.2. In embodiments, the solid form of Compound A tris(hydroxymethyl)aminomethane salt has an X-ray diffraction pattern with eighteen characteristic peaks expressed in values of degrees 2θ at 3.7±0.2, 7.5±0.2, 11.2±0.2, 14.9±0.2, 17.8±0.2, 18.8±0.2, 19.0±0.2, 19.7±0.2, 20.6±0.2, 21.5±0.2, 22.4±0.2, 22.6±0.2, 25.0±0.2, 25.7±0.2, 26.4±0.2, 27.8±0.2, 28.7±0.2, and 29.3±0.2. In embodiments, the solid form of Compound A tris(hydroxymethyl)aminomethane salt has an X-ray diffraction pattern with nineteen characteristic peaks expressed in values of degrees 2θ at 3.7±0.2, 7.5±0.2, 11.2±0.2, 14.9±0.2, 17.8±0.2, 18.8±0.2, 19.0±0.2, 19.7±0.2, 20.6±0.2, 21.5±0.2, 22.4±0.2, 22.6±0.2, 25.0±0.2, 25.7±0.2, 26.4±0.2, 27.8±0.2, 28.7±0.2, 2 and 9.2±0.2, 29.3±0.2. In embodiments, the solid form of Compound A tris(hydroxymethyl)aminomethane salt has an X-ray diffraction pattern with twenty characteristic peaks expressed in values of degrees 28 at 3.7±0.2, 7.5±0.2, 11.2±0.2, 15.0±0.2, 17.8±0.2, 18.4±0.2, 18.8±0.2, 19.0±0.2, 19.7±0.2, 20.6±0.2, 21.5±0.2, 22.4±0.2, 22.6±0.2, 25.0±0.2, 25.7±0.2, 26.4±0.2, 27.8±0.2, 28.7±0.2, 29.2±0.2, and 29.3±0.2.

In embodiments, the solid form of Compound A tris(hydroxymethyl)aminomethane salt has an X-ray diffraction pattern with at least three (such as at least four, five, six, seven, eight, nine, ten, eleven, twelve, thirteen, fourteen, fifteen, sixteen, seventeen, eighteen, nineteen, or twenty) characteristic peaks expressed in values of degrees 2θ selected from those at 3.7±0.2, 7.5±0.2, 11.2±0.2, 15.0±0.2, 17.8±0.2, 18.4±0.2, 18.8±0.2, 19.0±0.2, 19.7±0.2, 20.6±0.2, 21.5±0.2, 22.4±0.2, 22.6±0.2, 25.0±0.2, 25.7±0.2, 26.4±0.2, 27.8±0.2, 28.7±0.2, 29.2±0.2, and 29.3±0.2.

In embodiments, the solid form of Compound A tris(hydroxymethyl)aminomethane salt exhibits a differential scanning calorimetry thermogram having an endotherm onset value at 162.9±2° C.

In embodiments, the solid form of Compound A tris(hydroxymethyl)aminomethane salt exhibits a differential scanning calorimetry thermogram having an endotherm onset value at 159.8±2° C.

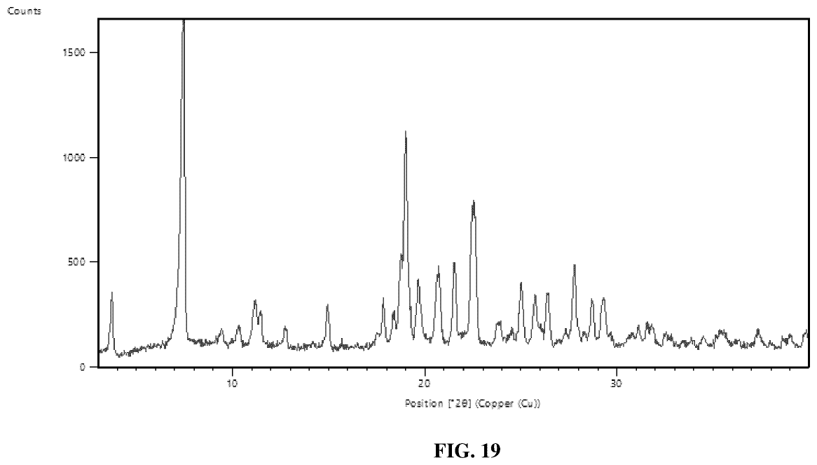

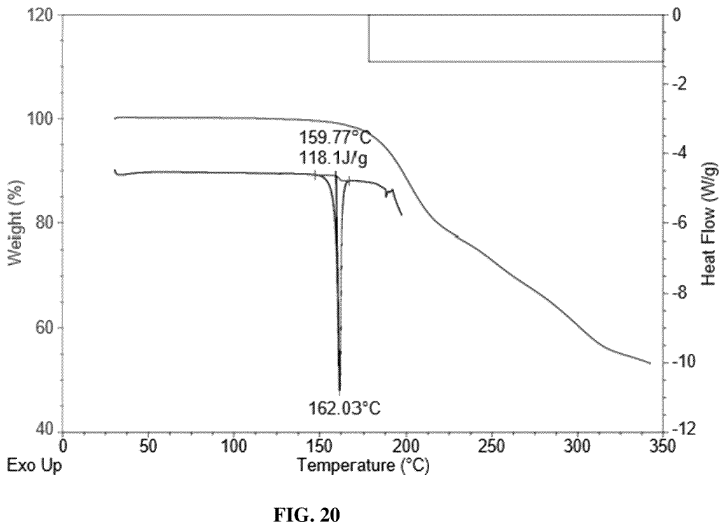

In embodiments, the solid form of Compound A tris(hydroxymethyl)aminomethane salt exhibits a thermogravimetric analysis thermogram/differential scanning calorimetry thermogram pattern substantially similar to that of FIG. 20.

In embodiments, the pharmaceutically acceptable salt of Compound A is hydrochloride salt. In further embodiments, Compound A hydrochloride salt is in a solid form.

In embodiments, the solid form of Compound A hydrochloride salt is Compound A hydrochloride salt Type A. In embodiments, Compound A hydrochloride salt Type A has an X-ray diffraction pattern with at least three (such as at least four, five, six, seven, eight, nine, ten, eleven, twelve, or thirteen) characteristic peaks expressed in values of degrees 2θ selected from those at 7.1±0.2; 7.9±0.2, 10.0±0.2, 12.9±0.2, 17.9±0.2, 19.3±0.2, 20.9±0.2, 21.5±0.2, 22.7±0.2, 25.1±0.2, 26.0±0.2, 27.0±0.2, and 30.7±0.2.

In embodiments, the solid form of Compound A hydrochloride salt is Compound A hydrochloride salt Type B. In embodiments, Compound A hydrochloride salt Type B has an X-ray diffraction pattern with at least three (such as at least four, five, or six,) characteristic peaks expressed in values of degrees 2θ selected from those at 6.9±0.2, 8.2±0.2, 15.0±0.2, 19.8±0.2, 23.8±0.2, and 24.7±0.2.

In embodiments, the solid form of Compound A hydrochloride salt is Compound A hydrochloride salt Type C. In embodiments, Compound A hydrochloride salt Type C has an X-ray diffraction pattern with at least three (such as at least four, five, six, seven, eight, or nine) characteristic peaks expressed in values of degrees 2θ selected from those at 6.1±0.2, 9.2±0.2, 18.4±0.2, 19.5±0.2, 21.8±0.2, 22.5±0.2, 23.8±0.2, and 25.0±0.2, 28.4±0.2.

In embodiments, the solid form of Compound A hydrochloride salt is Compound A hydrochloride salt Type D. In embodiments, Compound A hydrochloride salt Type D has an X-ray diffraction pattern with at least three (such as at least four, five, six, or seven) characteristic peaks expressed in values of degrees 2θ selected from those at 4.9±0.2, 6.9±0.2, 9.5±0.2, 19.8±0.2, 22.0±0.2, 25.1±0.2, and 25.9±0.2.

In embodiments, the solid form of Compound A hydrochloride salt comprises two or more solid forms selected from Compound A hydrochloride salt Type A, Compound A hydrochloride salt Type B, Compound A hydrochloride salt Type C, and Compound A hydrochloride salt Type D.

In embodiments, the pharmaceutically acceptable salt of Compound A is sulfate. In further embodiments, Compound A sulfate is in a solid form.

In embodiments, the solid form of Compound A sulfate has an X-ray diffraction pattern with at least three (such as at least four, five, six, seven, eight, or nine) characteristic peaks expressed in values of degrees 2θ selected from those at 3.4±0.2, 5.7±0.2, 6.9±0.2, 9.0±0.2, 16.8±0.2, 18.2±0.2, 20.1±0.2, 21.0±0.2, and 24.6±0.2.

In embodiments, the pharmaceutically acceptable salt of Compound A is napadisylate. In further embodiments, Compound A napadisylate is in a solid form.

In embodiments, the solid form of Compound A napadisylate has an X-ray diffraction pattern with at least three (such as at least four, five, six, seven, eight, nine, or ten) characteristic peaks expressed in values of degrees 2θ selected from those at 6.7±0.2, 8.0±0.2, 11.6±0.2, 14.7±0.2, 15.3±0.2, 15.5±0.2, 17.5±0.2, 18.6±0.2, 20.4±0.2, 20.8±0.2, 23.0±0.2, 23.4±0.2, 23.8±0.2, 24.3±0.2, 24.8±0.2, 26.7±0.2, 28.6±0.2, and 29.9±0.2.

In embodiments, the pharmaceutically acceptable salt of Compound A is mesylate. In further embodiments, Compound A mesylate is in a solid form.

In embodiments, the solid form of Compound A mesylate has an X-ray diffraction pattern with characteristic peaks expressed in values of degrees 2θ with at least three (such as at least four, five, six, seven, eight, nine, or ten) characteristic peaks expressed in values of degrees 2θ selected from those at 5.8±0.2, 11.3±0.2, 16.4±0.2, 19.8±0.2, 20.4±0.2, 21.8±0.2, 22.8±0.2, 23.3±0.2, 25.1±0.2, 25.9±0.2, and 29.7±0.2.

In embodiments, the pharmaceutically acceptable salt of Compound A is napsylate. In further embodiments, Compound A napsylate is in a solid form.

In embodiments, the solid form of Compound A napsylate has an X-ray diffraction pattern with at least three (such as at least four, five, six, seven, eight, nine, or ten) characteristic peaks expressed in values of degrees 2θ selected from those at 9.0±0.2, 12.0±0.2, 13.0±0.2, 13.3±0.2, 15.2±0.2, 16.3±0.2, 16.8±0.2, 19.1±0.2, 19.8±0.2, 21.1±0.2, 22.0±0.2, 22.8±0.2, 23.7±0.2, 24.0±0.2, 24.2±0.2, 24.9±0.2, 25.7±0.2, 26.9±0.2, 27.4±0.2, 29.4±0.2, and 30.4±0.2.

In embodiments, the pharmaceutically acceptable salt of Compound A is besylate. In further embodiments, Compound A besylate is in a solid form.

In embodiments, the solid form of Compound A besylate has an X-ray diffraction pattern with at least three (such as at least four, five, six, seven, eight, nine, or ten) characteristic peaks expressed in values of degrees 2θ selected from those at 3.9±0.2, 13.3±0.2, 13.9±0.2, 14.8±0.2, 15.5±0.2, 16.3±0.2, 19.1±0.2, 19.7±0.2, 21.9±0.2, 24.9±0.2, 26.0±0.2, 26.9±0.2, and 27.6±0.2.

In embodiments, the pharmaceutically acceptable salt of Compound A is sodium salt. In further embodiments, Compound A sodium salt is in a solid form.

In embodiments, the solid form of Compound A sodium salt has an X-ray diffraction pattern with at least three (such as at least four, five, six, or seven) characteristic peaks expressed in values of degrees 2θ selected from those at 3.2±0.2, 9.7±0.2, 13.2±0.2, 14.6±0.2, 16.2±0.2, 18.1±0.2, and 23.6±0.2.

In embodiments, the pharmaceutically acceptable salt of Compound A is potassium salt.

In further embodiments, Compound A potassium salt is in a solid form.

In embodiments, the solid form of Compound A potassium salt has an X-ray diffraction pattern with at least three (such as at least four, five, six, or seven) characteristic peaks expressed in values of degrees 2θ selected from those at 6.2±0.2, 9.4±0.2, 12.6±0.2, 15.9±0.2, 19.0±0.2, 24.0±0.2, and 37.7±0.2.

In embodiments, the pharmaceutically acceptable salt of Compound A is L-arginine salt.

In further embodiments, Compound A L-arginine salt is in a solid form.

In embodiments, the solid form of Compound A L-arginine salt has an X-ray diffraction pattern with at least three (such as at least four, five, six, seven, eight, nine, or ten) characteristic peaks expressed in values of degrees 2θ selected from those at 6.3±0.2, 9.3±0.2, 12.7±0.2, 18.6±0.2, 19.1±0.2, 19.6±0.2, 21.0±0.2, 21.5±0.2, 22.0±0.2, 22.8±0.2, 23.9±0.2, 24.1±0.2, 24.9±0.2, 25.7±0.2, 27.3±0.2, 27.6±0.2, 28.1±0.2, 29.8±0.2, and 30.3±0.2.

In embodiments, the pharmaceutically acceptable salt of Compound A is choline salt. In further embodiments, Compound A choline salt is in a solid form.

In embodiments, the solid form of Compound A choline salt has an X-ray diffraction pattern with at least three (such as at least four, five, six, seven, eight, nine, or ten) characteristic peaks expressed in values of degrees 2θ selected from those at 5.6±0.2, 8.4±0.2, 11.4±0.2, 14.4±0.2, 15.3±0.2, 17.1±0.2, 20.3±0.2, 24.0±0.2, 25.9±0.2, and 28.8±0.2.

In embodiments, the pharmaceutically acceptable salt of Compound A is L-lysine salt. In further embodiments, Compound A L-lysine salt is in a solid form.

In embodiments, the solid form of Compound A L-lysine has an X-ray diffraction pattern with at least three (such as at least four, five, six, seven, eight, nine, or ten) characteristic peaks expressed in values of degrees 2θ selected from those at 5.2±0.2, 7.9±0.2, 8.7±0.2, 12.2±0.2, 13.6±0.2, 15.4±0.2, 17.3±0.2, 20.1±0.2, 21.8±0.2, 22.6±0.2, 24.0±0.2, 25.1±0.2, and 27.5±0.2.

In embodiments, the pharmaceutically acceptable salt of Compound A is meglumine salt. In further embodiments, Compound A meglumine salt is in a solid form.

In embodiments, the solid form of Compound A meglumine salt has an X-ray diffraction pattern with at least three (such as at least four, five, six, seven, eight, nine, ten, or eleven) characteristic peaks expressed in values of degrees 2θ selected from those at 3.4±0.2, 5.6±0.2, 8.8±0.2, 11.3±0.2, 14.0±0.2, 17.1±0.2, 20.5±0.2, 22.0±0.2, 24.0±0.2, 26.5±0.2, and 28.8±0.2.

In an embodiment, the solid form of any one of the previous embodiments is a single solid form substantially free of other solid forms.

In certain aspects, embodiments herein relate to a pharmaceutical composition comprising an effective amount of a compound as contemplated herein and a pharmaceutically acceptable excipient.

In further aspects, embodiments herein relate to a method of antagonizing cyclic GMP-AMP synthase (cGAS) in a patient in need thereof, comprising administering an effective amount of a compound as contemplated herein or a pharmaceutical composition thereof.

In still further aspects, embodiments herein relate to a method of treating an inflammatory, allergic, autoimmune, or neurodegenerative disease in a patient in need thereof, comprising administering an effective amount of a compound as contemplated herein or a pharmaceutical composition thereof.

In another aspect, embodiments herein relate to use of a compound as contemplated herein or a pharmaceutical composition thereof in the manufacture of a medicament for antagonizing cyclic GMP-AMP synthase (cGAS) in a patient in need thereof.

In still another aspect, embodiments herein relate to use of a compound as contemplated herein or a pharmaceutical composition thereof in the manufacture of a medicament for treating an inflammatory, allergic, autoimmune, or neurodegenerative disease in a patient in need thereof.

In an aspect, embodiments herein relate to a compound as contemplated herein or a pharmaceutical composition thereof for use in therapy, such as for treating an inflammatory, allergic, autoimmune, cardiovascular, or neurodegenerative disease in a patient, and/or for antagonizing cyclic GMP-AMP synthase (cGAS) in a patient.

In particular, the present disclosure provides quinoline cGAS antagonist compounds, which find utility as inhibitors of cGAS. An advantage of the compounds provided herein is that a broad range of pharmacological activities is possible, consistent with the inhibition of cGAS. In addition, the disclosure provides methods of using the solid forms and compositions thereof described herein for the treatment of inflammatory, allergic, autoimmune, and infectious diseases. The solid forms and compositions thereof can also be used for the treatment of senescence- or age-related diseases, such as neurodegenerative diseases, cardiovascular diseases, liver and renal diseases, cancer, and premature aging.

### 5. EXAMPLES

The compounds, the solid forms, the salts of the compounds, and the solid forms of the salts of the compounds of the present disclosure can be prepared in a number of ways known to one skilled in the art of organic synthesis. The compounds of the present disclosure can be synthesized using the methods described below, together with synthetic methods known in the art of synthetic organic chemistry, or by variations thereon as appreciated by those skilled in the art. Preferred methods include, but are not limited to, those described below. The reactions are performed in a solvent or solvent mixture appropriate to the reagents and materials employed and suitable for the transformations being effected. It will be understood by those skilled in the art of organic synthesis that the functionality present on the molecule should be consistent with the transformations proposed. This will sometimes require a judgment to modify the order of the synthetic steps or to select one particular process scheme over another in order to obtain a desired compound of the disclosure.

### 5.1. Characterization of Starting Material

### 5.1.1.1 Baseline Characterization

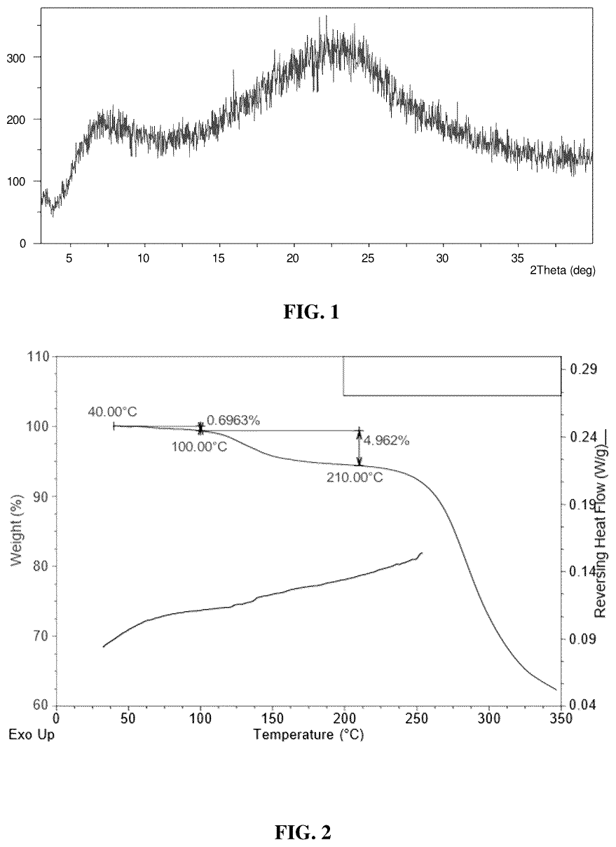

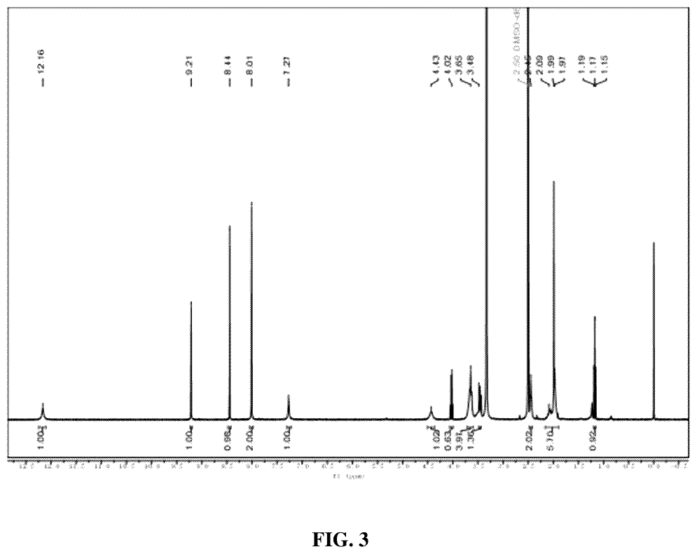

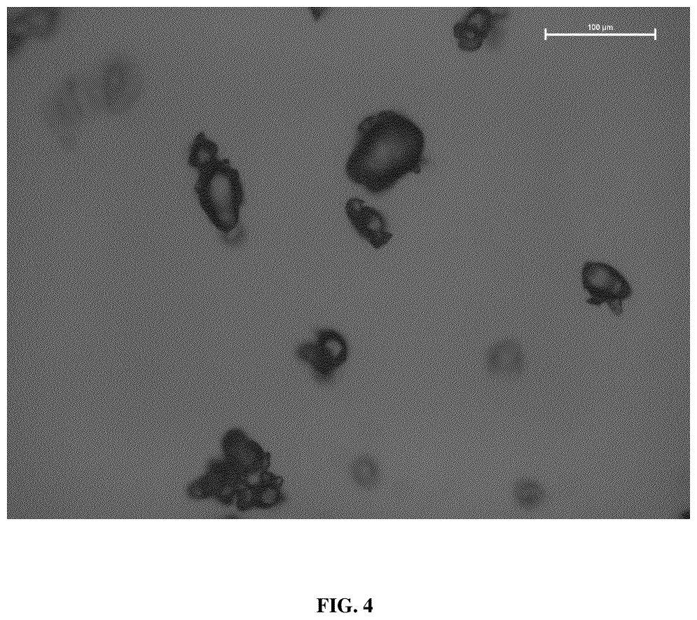

The starting freeform material, Compound A, was characterized by XRPD/TGA/mDSC/¹H NMR/PLM. The XRPD diffractogram showed that the sample was amorphous as shown in FIG. 1. The TGA result (FIG. 2) showed a weight loss of 0.7 wt % before 100° C. and 5.0 wt % from 100° C. to 210° C. The ¹H NMR spectrum (DMSO-d₆) (FIG. 3) was consistent with the structure and approximately 5.2 wt % of residual EtOAc was observed. The PLM image showed bulky particles with no birefringence (FIG. 4). ¹HNMR (400 MHz, DMSO-d₆): 12.16 (s, 1H), 9.21 (s, 1H), 8.44 (s, 1H), 8.01 (s, 2H), 7.21 (s, 1H), 4.43 (bs, 1H), 3.65 (m, 4H), 3.48 (m, 2H), 2.50-2.45 (m, 2H), 2.09-1.97 (m, 4H) ppm.

### 5.1.1.2 Instruments and Methods

### 5.1.1.2.1 X-ray Powder Diffraction (XRPD)

X-ray powder diffraction data was collected under ambient conditions on a BRUKER D2 PHASER diffractometer. Approximately 2 mg of sample was placed on a silicon sample holder and flattened by a glass slide with protection of a piece of weighting paper. An X-ray generator of 300 W was employed at 30 kV and 10 mA and was equipped with an X-ray tube of Cu(Kα), with the Kα2/Kα1 intensity ratio of 0.50 (1.54439 Å/1.5406 Å). The divergence slit was 0.6 mm and soller slit was 4°. Data were collected at a speed of 0.15 s/step with step size of 0.02° (20) and a total step of 1837.

X-ray powder diffraction data was collected under ambient conditions on a Malvern Panalytical Aeris diffractometer. A few milligrams of sample were placed on a silicon sample holder and flattened by a glass slide with protection of a piece of weighting paper. An X-ray generator of 300 W was employed at 40 kV and 7.5 mA and was equipped with an X-ray tube of Cu(Kα), with the Kα2/Kα1 intensity ratio of 0.50 (1.54439 Å/1.5406 Å). Data were collected from 3° to 400 (20) at a scan speed of 0.14°/s with step size of 0.02° (20).

### 5.1.1.2.2 Thermogravimetric Analysis

Thermogravimetric analysis data were collected with a TA® Discovery 550/5500 series TGA. A few milligrams of sample were added to a Tzero aluminum pan and heated from room temperature to the target temperature with a heating rate of 10° C. per minute under nitrogen (flow rate of TA Discovery 5500: 25 mL/min, flow rate of TA Discovery 550: 60 mL/min).

### 5.1.1.2.3 Differential Scanning Calorimetry

Differential scanning calorimetry was performed with a TA® Discovery 2500 series DSC using a few milligrams of material in a Tzero aluminum pan sealed with a Tzero hermetic lid. Samples were analyzed using a heating rate of 10° C. per minute under 50 mL per minute of nitrogen flow.

### 5.1.1.2.4 Polarized Light Microscopy

Photomicrographs were taken using a Nikon® ECLIPSE Ci-POL polarized light microscope at room temperature equipped with eyepieces of 10× and objective of 5/20/50/100× magnification. Sample (<1 mg) was dispersed with a drop of oil on a glass slide and photomicrograph was taken.

### 5.1.1.2.5 Proton Nuclear Magnetic Resonance

¹H NMR data was taken using BRUKER AVANCE NEO 400 MHz in DMSO-d₆ or CD₃OD.

### 5.1.1.2.6 Ion Chromatography (IC)

IC of DIONEX™ ICS-6000⁺ DP was employed to detect the SO₄²⁻ and Cl⁻ content.

### 5.1.1.2.7 Inductive Coupled Plasma (ICP)

PerkinElmer Avio® 200 ICP-OES was employed to detect the assay of potassium ions. Multiwave 7000 ICP-MS was employed to detect the assay of sodium ions.

### 5.1.1.2.8 High Performance Liquid Chromatography

SHIMADZU LC-20AD was employed to detect the chemical purity and solubility, with the methods shown in Table 1.

TABLE 1
HPLC method for purity and solubility test

| Instrument | SHIMADZU LC-20AD | | |
|---|---|---|---|
| Column | Agilent Poroshell 120 EC-C₁₈ | | |
| | (4.6 mm × 100 mm, 2.7 μm) | | |
| Column temp. | 40° C. | | |
| Mobile phase | A: 2.0% formic acid | B: ACN | C: 0.1% IPA |
| | in H₂O | | in H2O |

| Detection wavelength | 250 nm | | | |
|---|---|---|---|---|
| Flow Rate | 0.8 mL/min | | | |
| Injection | 5 μL | | | |
| Diluent | 50% ACN in H₂O | | | |
| Conc. | 0.3 mg/mL | | | |
| Gradient | Time (min) | A % | B % | C % |
| | 0 | 5 | 10 | 85 |
| | 0.50 | 5 | 10 | 85 |
| | 12.00 | 5 | 95 | 0 |
| | 15.00 | 5 | 95 | 0 |

### 5.1.1.2.9 Dynamic Vapor Sorption

Dynamic vapor sorption was performed with ADVENTURE series DVS and Intrinsic-1 DVS at 25° C. under nitrogen. Approximately 30 milligrams of material were used. The anhydrate (as applicable) was analyzed using methods below: 0% relative humidity (RH) to 95% RH to 0% RH at 10% RH (5% RH from 90% RH to 95% RH to 90% RH).

### 5.1.1.3 Approximate Solubility

The solubility of the starting freeform material was estimated in 20 different solvents at room temperature (˜26° C.). Specifically, approximately 2 mg of solids were added into separate HPLC vials. Solvents in Table 2 were then added stepwise (50/50/200/700 L) into the vials until the solids were dissolved or a total volume of 1.0 mL was reached.

TABLE 2
Approximate solubility of the starting material at room temperature

| | Solubility | | Solubility |
|---|---|---|---|
| Solvent | (mg/mL) | Solvent | (mg/mL) |
| MeOH | S > 44.4 | THF | S > 36.2 |
| EtOH | S > 40.4 | 2-MeTHF | S > 38.6 |
| IPA | S > 39.6 | 1,4-Dioxane | S > 37.4 |
| Acetone | S > 38.4 | ACN | S < 2.1 |
| MEK | S > 40.2 | DCM | S > 46.2 |
| MIBK | S > 36.4 | CHCl₃ | S > 40.2 |
| EtOAc | S > 43.2 | DMSO | S > 39.0 |
| IPAc | S > 41.0 | Toluene | 21.3 < S < 42.6 |
| MTBE | S > 36.8 | n-Heptane | S < 1.8 |
| CPME | S > 45.2 | H₂O | S < 2.0 |

### 5.1.2. Preparation of Salts

Based on the pKa values (0.6, 3.0, basic; 4.3, acidic) and approximate solubility of Compound A as the free acid, a total of 120 salt experiments were conducted with a matrix of 27 acids or bases, also referred to as “formers,” and four solvent systems with charge molar ratio 1:1 or 2:1 (former/freeform), using the amorphous freeform as the starting material. Specifically, (1) for column A/B/C, ˜40 mg/mL stock solution of freeform was prepared, and then 0.5 mL was distributed to each HPLC vial containing the corresponding former; (2) for column D, ˜20 mg of freeform and corresponding solid former was weighed in an HPLC vial, and 0.5 mL of corresponding solvent was added (liquid formers were added after solvents). All experiments were magnetically stirred at r.t. for ˜2 days, the obtained suspensions/solutions were post-treated following: (1) any solids that were isolated by centrifugation, (2) clear solution was transferred to 5° C. If still no solid was obtained, the clear solution was subjected to an anti-solvent (n-heptane for column A/B/C or methyl tert-butyl ether (MTBE) for column D) addition or evaporation at r.t., (3) gel/oil samples were subjected to stirring along with temperature cycling (50° C.-5° C.-50° C., 0.1° C./min, at least 3 cycles) to induce crystallization. The isolated solids were dried in vacuo at 40° C. overnight before XRPD analysis. Samples with low crystallinity were further treated by temperature cycling slurry in mother liquor to improve crystallinity.

As summarized in Table 3, a total of 2 crystal forms of the freeform and 17 salts were identified, which were named as freeform Type A/B, HCl salt Type A/B/C, sulfate Type A, napadisylate Type A, esylate Type A, mesylate Type A, napsylate Type A, besylate Type A, sodium salt Type A, potassium salt Type A, L-arginine salt Type A, choline Type A, L-lysine salt Type A, t-butylamine salt Type A, meglumine salt Type A and tris salt Type A, respectively in the below table. A new form of HCl salt, namely HCl salt Type D, was observed in re-preparation of HCl salt. The representative batches were further characterized by TGA/DSC/¹H NMR/PLM/HPLC, and the results were summarized in Table 4.

TABLE 3
Summary of Salt Experiments

| Salt Formers | | | | | | | D |
|---|---|---|---|---|---|---|---|
| (molar charge ratio, | | Safety | | A | B | C | ACN/H₂O |
| former/freeform) | | Class | pKa | MEK | EtOAc | 2-MeTHF | (9:1, v/v) |
| 0 | Blank | N/A | N/A | Freeform Type | Freeform Type | Gel⁴ | Gel⁴ |
| | | | | A⁴ | B⁴ | | |
| 1 | HCl (2:1) | 1 | −6.0 | Gel¹ | Gel¹ | HCl salt Type A¹ | HCl salt Type A⁵ |
| 29 | HCl (1:1) | 1 | −6.0 | HCl salt Type B | Gel¹ | HCl salt Type C | HCl salt Type A⁴ |
| 2 | Sulfuric acid (2:1) | 1 | −3.0 | Amorphous | Amorphous | Gel¹ | Gel⁴ |
| 30 | Sulfuric acid (1:1) | 1 | −3.0 | Sulfate Type A | Gel¹ | Gel¹ | Gel⁴ |
| 3 | Maleic acid | 1 | 1.9 | Gel⁴ | Gel² | Gel⁴ | Gel⁴ |
| 4 | Phosphoric acid | 1 | 2.0 | Gel¹ | Gel¹ | Gel⁴ | Gel⁴ |
| 5 | Fumaric acid | 1 | 3.0 | Freeform Type | Freeform Type | Gel⁴ | Gel⁴ |
| | | | | A + Fumaric acid¹ | B + Fumaric acid⁶ | | |
| 6 | Citric acid | 1 | 3.1 | Gel⁴ | Gel² | Gel⁴ | Gel⁴ |
| 7 | Naphthalene-1,5- | 2 | −3.4, −2.6 | Amorphous¹ | Naphthalene-1,5- | Amorphous | Napadisylate |
| | disulfonic acid | | | | disulfonic | | Type A |
| | | | | | acid + peaks³ | | |
| 8 | Ethane-1,2- | 2 | −2.1, −1.5 | Gel¹ | Gel¹ | Gel¹ | Gel² |
| | disulfonic acid | | | | | | |
| | (1:1) | | | | | | |
| 9 | Ethane-1,2- | 2 | −2.1, −1.5 | Gel¹ | Gel¹ | Gel¹ | Gel² |
| | disulfonic acid | | | | | | |
| | (2:1) | | | | | | |
| 10 | Ethanesulfonic | 2 | −2.0 | Esylate Type A | Esylate Type A | Esylate Type A | Gel⁴ |
| | acid | | | | | | |
| 11 | p-Toluenesulfonic | 2 | −1.3 | Gel^{2,8} | Gel^{1,8} | Gel^{1,8} | Gel⁴ |
| | acid (1:1) | | | | | | |
| 12 | Methanesulfonic | 2 | −1.2 | Mesylate Type A | Gel^{1,8} | Gel^{1,8} | Gel⁴ |
| | acid | | | | | | |
| 13 | Naphthalene-2- | 2 | 0.2 | Napsylate Type A | Napsylate Type A | Gel¹ | Gel⁴ |
| | sulfonic acid | | | | | | |
| 14 | Benzenesulfonic | 2 | 0.7 | Besylate Type A | Gel¹ | Gel¹ | Gel⁴ |
| | acid | | | | | | |
| 15 | Oxalic acid | 2 | 1.3 | Gel⁴ | Gel¹ | Gel⁴ | Gel⁴ |
| 16 | Sodium hydroxide | 1 | ~14 | Amorphous¹ | Amorphous¹ | Amorphous¹ | Sodium salt Type |
| | | | | | | | A⁷ |
| 17 | Potassium | 1 | ~14 | Amorphous^{1,3} | Potassium salt | Potassium salt | Gel¹ |
| | hydroxide | | | | Type A^{1,3} | Type A | |
| 18 | Magnesium | 1 | ~14 | Freeform Type | Freeform Type | Gel⁴ | Magnesium |
| | hydroxide | | | A + Magnesium | B + Magnesium | | hydroxide + peaks³ |
| | | | | hydroxide⁴ | hydroxide⁶ | | |
| 19 | L-Arginine | 1 | 13.2 | Gel¹ | L-Arginine | Gel¹ | L-Arginine salt |
| | | | | | | | Type A |
| 20 | Calcium | 1 | 12.6 | Calcium | Calcium | Calcium | Calcium |
| | hydroxide | | | hydroxide³ | hydroxide³ | hydroxide³ | hydroxide + peaks³ |
| 21 | Choline | 1 | >11 | Choline salt | Gel¹ | Gel¹ | Gel⁴ |
| | | | | Type A | | | |
| 22 | L-Lysine | 1 | 10.8 | Amorphous | L-Lysine salt | Low crystallinity³ | Gel¹ |
| | | | | | Type A³ | | |
| 23 | t-Butylamine | 1 | 10.7 | t-Butylamine salt | t-Butylamine salt | t-Butylamine salt | t-Butylamine salt |
| | | | | Type A | Type A | Type A | Type A |
| 24 | Ammonia | 1 | 9.3 | Gel⁴ | Gel¹ | Gel⁴ | Gel⁵ |
| 25 | N-Methyl | 1 | 8.0 | Meglumine salt | Gel¹ | Gel¹ | Gel¹ |
| | glucamine | | | Type A | | | |
| 26 | Deanol | 2 | 8.8 | Gel⁴ | Gel⁴ | Gel⁴ | Gel² |
| 27 | Tromethamine | 2 | 8.0 | Tris salt Type A | Tris salt Type | Tris salt Type A | Gel¹ |
| | (Tris) | | | | A + tromethamine | | |
| 28 | L-Proline | — | — | Gel⁴ | Gel⁴ | L-Proline⁴ | Gel² |

¹Gel was obtained after slurry at r.t. for 2 days. Transfer to temperature cycling slurry.
²Clear solution or limited solids were obtained after slurry at r.t. for 2 days. Transfer to slurry at 5° C. and gel or oil was obtained. Transfer to temperature cycling slurry.
³Add the mother liquor back followed by temperature cycling slurry.
⁴Clear solution or limited solids were obtained after slurry at r.t./5° C. Add anti-solvent (n-heptane for column A/B/C or MTBE for column D) and slurry at 5° C. Clear solution or limited solids were obtained. Transfer to evaporate at RT and gel was obtained.
⁵Clear solution or limited solids were obtained after slurry at r.t./5° C. Add anti-solvent (n-heptane for column A/B/C or MTBE for column D) and gel/oil was obtained. Transfer to temperature cycling slurry.
⁶Clear solution or limited solids were obtained after slurry at r.t./5° C. Add n-heptane as anti-solvent, and solids were obtained after slurry at 5° C.
⁷Add the mother liquor back and transfer to temperature cycling slurry, and limited solids were obtained. Transfer to evaporate at r.t..
⁸Gel was obtained after slurry at RT for 2 days. One more equivalent acid was added, and stir for another 3 days at r.t.. The samples were still gel.

TABLE 4
Summary of Characterization of Solid forms

| | | | Weight Loss | | Endotherms | | |
|---|---|---|---|---|---|---|---|
| | | | in TGA | | in DSC | Stoichiometry | |
| Crystal | | | (%, end | | (° C., peak | (former/ | Purity |
| forms/salts | | Crystallinity | temperature) | | temperature) | freeform) | (%) |
| Freeform | Type A | High | 6.4 | (100° C.) | 82.9 | N/A | 97.71 |
| | | | 5.2 | (200° C.) | | | |
| | Type B | High | Negligible | | 144.3 | N/A | 97.53 |
| | | | before | | | | |
| | | | decomposition | | | | |
| HCl salt | Type A | High | 6.3 | (150° C.) | 122.4 | 1.0 | 98.89 |
| | Type B | High | Negligible | | 170.2, 179.6 | 1.0 | 97.67 |
| | Type C | Medium | 0.8 | (90° C.) | 129.2, 152.1, 185.1 | 0.9 | 96.41 |
| | | | 6.6 | (140° C.) | | | |
| | Type D | Medium | 6.9 | (130° C.) | 99.2, 115.7 | 0.9 | — |

| Sulfate Type A | Low | 1.5 | (90° C.) | 79.2, 118.3, | 1.0 | 96.95 |
|---|---|---|---|---|---|---|
| | | 2.8 | (170° C.) | 134.4, 147.6 | | |
| Napadisylate | High | 3.4 | (150° C.) | 164.7, 223.1 | 1.0 | 98.38 |
| Type A | | | | | | |
| Esylate Type A | High | Negligible | | 211.8 | 1.0 | 98.15 |
| | | before | | | | |
| | | decomposition | | | | |
| Mesylate Type A | High | 1.3 | (100° C.) | 74.9, 127.7 | 1.0 | 98.58 |
| Napsylate Type A | High | 0.3 | (150° C.) | 118.3, 196.3 | 1.0 | 98.22 |
| Besylate Type A | Medium | 0.5 | (150° C.) | 170.4, 176.5 | 1.0 | 97.74 |
| Sodium salt Type A | Low | 15.3 | (160° C.) | 76.9 | 0.9 | — |
| Potassium salt | Low | 5.2 | (150° C.) | 63.6, 121.2 | 1.8 | 97.16 |
| Type A | | | | | | |
| L-Arginine salt | High | 3.1 | (180° C.) | 116.7, 151.1 | 1.0 | 98.32 |
| Type A | | | | | | |
| Choline salt Type A | Medium | 7.0 | (100° C.) | 69.0 | 1.0 | 98.62 |
| L-Lysine salt | Low | 1.7 | (150° C.) | 194.4 | 0.6 | 98.22 |
| Type A | | | | | | |
| t-Butylamine salt | High | 0.2 | (90° C.) | 168.6 | 1.0 | 97.76 |
| Type A | | 10.0 | (150° C.) | | | |
| Meglumine salt | Medium | 3.2 | (150° C.) | 95.7, 162.3 | 1.3 | 98.48 |

| | | | | | |
|---|---|---|---|---|---|
| Type A | | | | | |
| Tris salt Type A | High | Negligible | 162.9 | 1.0 | 99.01 |

before
decomposition

TABLE 5
XRPD Peak List of Compound A freeform Type A

| | | FWHM | | Rel. |
|---|---|---|---|---|
| Pos. [°2θ] | Height [cts] | Left [°2θ] | d-spacing [Å] | Int. [%] |
| 7.3774 | 6008.81 | 0.1082 | 11.97314 | 100.00 |
| 8.8452 | 1471.48 | 0.1082 | 9.98928 | 24.49 |
| 10.6488 | 772.81 | 0.1299 | 8.30115 | 12.86 |
| 12.3826 | 218.06 | 0.1299 | 7.14241 | 3.63 |
| 14.8498 | 147.50 | 0.1299 | 5.96085 | 2.45 |
| 16.1701 | 720.89 | 0.1082 | 5.47697 | 12.00 |
| 17.2492 | 577.92 | 0.1082 | 5.13671 | 9.62 |
| 17.8569 | 326.37 | 0.1082 | 4.96324 | 5.43 |
| 18.4871 | 670.24 | 0.1299 | 4.79544 | 11.15 |
| 19.6153 | 316.04 | 0.1082 | 4.52209 | 5.26 |
| 20.3696 | 495.18 | 0.1299 | 4.35630 | 8.24 |
| 20.9534 | 392.07 | 0.1299 | 4.23623 | 6.52 |
| 21.6658 | 370.41 | 0.2598 | 4.09852 | 6.16 |
| 21.9637 | 938.63 | 0.1299 | 4.04361 | 15.62 |
| 22.4152 | 134.28 | 0.1299 | 3.96317 | 2.23 |
| 22.9441 | 1410.51 | 0.1299 | 3.87299 | 23.47 |
| 23.6017 | 356.50 | 0.1082 | 3.76655 | 5.93 |
| 24.4213 | 196.11 | 0.1299 | 3.64197 | 3.26 |
| 25.0401 | 1032.81 | 0.1299 | 3.55335 | 17.19 |
| 26.4123 | 731.81 | 0.1082 | 3.37177 | 12.18 |
| 26.9628 | 198.57 | 0.1732 | 3.30416 | 3.30 |
| 27.3547 | 182.92 | 0.1082 | 3.25771 | 3.04 |
| 27.7044 | 147.18 | 0.1082 | 3.21739 | 2.45 |
| 28.1376 | 442.82 | 0.1082 | 3.16882 | 7.37 |
| 28.8790 | 111.17 | 0.1299 | 3.08913 | 1.85 |
| 29.8044 | 109.45 | 0.1299 | 2.99529 | 1.82 |
| 30.1045 | 122.29 | 0.1082 | 2.96611 | 2.04 |
| 31.4658 | 124.87 | 0.1732 | 2.84082 | 2.08 |
| 32.2809 | 91.29 | 0.1515 | 2.77093 | 1.52 |
| 32.9351 | 172.12 | 0.1082 | 2.71737 | 2.86 |
| 33.3787 | 138.13 | 0.1732 | 2.68226 | 2.30 |
| 34.0508 | 60.09 | 0.1299 | 2.63084 | 1.00 |
| 34.5472 | 33.18 | 0.1732 | 2.59417 | 0.55 |
| 35.6763 | 66.55 | 0.1732 | 2.51461 | 1.11 |
| 36.1797 | 70.23 | 0.1299 | 2.48077 | 1.17 |
| 37.1855 | 96.32 | 0.1299 | 2.41595 | 1.60 |
| 37.6094 | 109.80 | 0.1299 | 2.38969 | 1.83 |
| 37.9075 | 148.99 | 0.1082 | 2.37158 | 2.48 |

TABLE 6
XRPD Peak List of Compound A freeform Type B

| | | FWHM | | Rel. |
|---|---|---|---|---|
| Pos. [°2θ] | Height [cts] | Left [°2θ] | d-spacing [Å] | Int. [%] |
| 6.2673 | 1504.43 | 0.1299 | 14.09130 | 68.45 |
| 8.7830 | 369.77 | 0.1082 | 10.05988 | 16.83 |
| 9.8746 | 2058.36 | 0.1299 | 8.95019 | 93.66 |
| 12.6741 | 721.54 | 0.1082 | 6.97878 | 32.83 |
| 16.0814 | 1004.75 | 0.1299 | 5.50700 | 45.72 |
| 17.6954 | 641.60 | 0.1299 | 5.00818 | 29.19 |
| 18.2755 | 969.47 | 0.1082 | 4.85049 | 44.11 |
| 19.0880 | 452.91 | 0.1299 | 4.64582 | 20.61 |
| 19.9396 | 465.25 | 0.1082 | 4.44928 | 21.17 |
| 20.3502 | 532.79 | 0.1299 | 4.36042 | 24.24 |
| 20.8200 | 117.53 | 0.1082 | 4.26309 | 5.35 |
| 21.6628 | 78.76 | 0.1299 | 4.09909 | 3.58 |
| 22.6628 | 2197.70 | 0.1299 | 3.92043 | 100.00 |
| 23.1099 | 231.91 | 0.1299 | 3.84558 | 10.55 |
| 24.0361 | 526.57 | 0.1299 | 3.69945 | 23.96 |
| 24.7025 | 150.67 | 0.1082 | 3.60114 | 6.86 |
| 25.5812 | 817.51 | 0.1299 | 3.47940 | 37.20 |
| 26.1729 | 537.33 | 0.1082 | 3.40206 | 24.45 |
| 26.5846 | 340.22 | 0.1515 | 3.35030 | 15.48 |
| 27.1032 | 146.89 | 0.1299 | 3.28737 | 6.68 |
| 27.6371 | 163.24 | 0.1299 | 3.22506 | 7.43 |
| 28.3115 | 383.24 | 0.1515 | 3.14975 | 17.44 |
| 29.1416 | 150.74 | 0.1299 | 3.06189 | 6.86 |
| 30.1617 | 133.71 | 0.1299 | 2.96061 | 6.08 |
| 30.7447 | 111.96 | 0.1082 | 2.90579 | 5.09 |
| 31.0998 | 97.97 | 0.1299 | 2.87342 | 4.46 |
| 31.6409 | 416.49 | 0.1299 | 2.82550 | 18.95 |
| 32.6113 | 288.39 | 0.1515 | 2.74361 | 13.12 |
| 33.6757 | 112.90 | 0.1299 | 2.65928 | 5.14 |
| 34.3648 | 30.43 | 0.3464 | 2.60752 | 1.38 |
| 35.7353 | 82.93 | 0.2165 | 2.51059 | 3.77 |
| 37.1620 | 156.19 | 0.1299 | 2.41742 | 7.11 |
| 38.3250 | 23.44 | 0.1299 | 2.34670 | 1.07 |
| 39.6564 | 45.05 | 0.1299 | 2.27092 | 2.05 |

TABLE 7
XRPD Peak List of HCl salt Type A

| | | FWHM | | Rel. |
|---|---|---|---|---|
| Pos. [°2θ] | Height [cts] | Left [°2θ] | d-spacing [Å] | Int. [%] |
| 3.4063 | 26.57 | 0.5196 | 25.91763 | 3.17 |
| 4.9536 | 139.78 | 0.1299 | 17.82486 | 16.67 |
| 7.0977 | 838.78 | 0.1299 | 12.44435 | 100.00 |
| 7.9273 | 616.21 | 0.1082 | 11.14379 | 73.47 |
| 10.0484 | 346.12 | 0.1082 | 8.79575 | 41.26 |
| 11.2567 | 83.29 | 0.1299 | 7.85413 | 9.93 |
| 12.8710 | 589.13 | 0.1299 | 6.87250 | 70.24 |
| 14.2865 | 132.15 | 0.2165 | 6.19459 | 15.75 |
| 14.7263 | 60.42 | 0.1732 | 6.01055 | 7.20 |
| 15.8756 | 97.39 | 0.1299 | 5.57791 | 11.61 |
| 17.9357 | 190.12 | 0.1082 | 4.94161 | 22.67 |
| 18.3302 | 116.55 | 0.1299 | 4.83613 | 13.90 |
| 18.6039 | 93.50 | 0.1299 | 4.76561 | 11.15 |
| 19.2605 | 261.47 | 0.1082 | 4.60460 | 31.17 |
| 19.9465 | 91.47 | 0.1299 | 4.44775 | 10.91 |
| 20.3121 | 81.05 | 0.1299 | 4.36851 | 9.66 |
| 20.9124 | 239.43 | 0.1082 | 4.24445 | 28.54 |
| 21.4917 | 201.66 | 0.2165 | 4.13133 | 24.04 |
| 22.7124 | 273.74 | 0.1299 | 3.91197 | 32.64 |
| 23.5401 | 78.22 | 0.1299 | 3.77626 | 9.33 |
| 24.0698 | 85.37 | 0.2165 | 3.69435 | 10.18 |
| 25.0557 | 166.85 | 0.1515 | 3.55117 | 19.89 |
| 25.6502 | 115.50 | 0.1299 | 3.47020 | 13.77 |
| 26.0476 | 161.62 | 0.1082 | 3.41815 | 19.27 |
| 26.3278 | 106.96 | 0.1299 | 3.38240 | 12.75 |
| 27.0298 | 154.36 | 0.1299 | 3.29613 | 18.40 |
| 28.1595 | 51.90 | 0.4330 | 3.16640 | 6.19 |
| 28.7424 | 65.92 | 0.1732 | 3.10351 | 7.86 |
| 29.0719 | 108.07 | 0.1299 | 3.06908 | 12.88 |
| 29.2726 | 98.39 | 0.1299 | 3.04848 | 11.73 |
| 29.7485 | 127.50 | 0.1299 | 3.00080 | 15.20 |
| 29.9903 | 137.48 | 0.1299 | 2.97714 | 16.39 |
| 30.7467 | 241.56 | 0.1082 | 2.90561 | 28.80 |
| 33.1470 | 74.74 | 0.2598 | 2.70048 | 8.91 |
| 33.5603 | 49.83 | 0.2165 | 2.66817 | 5.94 |
| 35.7847 | 38.35 | 0.3031 | 2.50724 | 4.57 |
| 36.3516 | 45.37 | 0.1732 | 2.46943 | 5.41 |
| 39.5002 | 25.22 | 0.1299 | 2.27954 | 3.01 |

TABLE 8
XRPD Peak List of HCl salt Type B

| | | FWHM | | Rel. |
|---|---|---|---|---|
| Pos. [°2θ] | Height [cts] | Left [°2θ] | d-spacing [Å] | Int. [%] |
| 6.9067 | 122.12 | 0.1732 | 12.78813 | 29.13 |
| 8.2193 | 244.86 | 0.1299 | 10.74849 | 58.41 |
| 9.5873 | 27.47 | 0.4330 | 9.21771 | 6.55 |
| 11.4879 | 52.51 | 0.1732 | 7.69658 | 12.53 |
| 13.9495 | 28.18 | 0.2598 | 6.34345 | 6.72 |
| 15.0411 | 84.42 | 0.1732 | 5.88547 | 20.14 |
| 16.4009 | 61.94 | 0.1299 | 5.40042 | 14.77 |
| 16.8287 | 53.00 | 0.1732 | 5.26409 | 12.64 |
| 17.8569 | 71.16 | 0.1732 | 4.96324 | 16.97 |
| 18.5964 | 41.15 | 0.1299 | 4.76750 | 9.82 |
| 19.0098 | 35.03 | 0.1299 | 4.66475 | 8.36 |
| 19.7621 | 168.13 | 0.1299 | 4.48884 | 40.11 |
| 20.6456 | 57.00 | 0.1515 | 4.29870 | 13.60 |
| 21.1416 | 42.52 | 0.1299 | 4.19895 | 10.14 |
| 21.6213 | 74.46 | 0.1299 | 4.10685 | 17.76 |
| 22.6298 | 27.18 | 0.2598 | 3.92607 | 6.48 |
| 23.1469 | 54.26 | 0.2165 | 3.83952 | 12.94 |
| 23.8206 | 155.02 | 0.1515 | 3.73242 | 36.98 |
| 24.7318 | 419.23 | 0.1299 | 3.59694 | 100.00 |
| 25.5622 | 66.73 | 0.1299 | 3.48194 | 15.92 |
| 27.0904 | 30.99 | 0.6927 | 3.28889 | 7.39 |
| 30.5723 | 26.93 | 0.2165 | 2.92179 | 6.42 |
| 33.7130 | 30.52 | 0.1299 | 2.65643 | 7.28 |

TABLE 9
XRPD Peak List of HCl salt Type C

| | | FWHM | | Rel. |
|---|---|---|---|---|
| Pos. [°2θ] | Height [cts] | Left [°2θ] | d-spacing [Å] | Int. [%] |
| 3.2309 | 26.23 | 0.6061 | 27.32452 | 1.43 |
| 6.1390 | 1836.28 | 0.1515 | 14.38543 | 100.00 |
| 9.2367 | 143.18 | 0.1299 | 9.56681 | 7.80 |
| 16.4935 | 22.23 | 0.3031 | 5.37031 | 1.21 |
| 17.0625 | 26.92 | 0.3464 | 5.19250 | 1.47 |
| 18.3856 | 81.03 | 0.2165 | 4.82170 | 4.41 |
| 19.4792 | 90.58 | 0.1299 | 4.55339 | 4.93 |
| 20.8071 | 60.78 | 0.2165 | 4.26570 | 3.31 |
| 21.7595 | 142.65 | 0.1299 | 4.08109 | 7.77 |
| 22.5108 | 162.61 | 0.1732 | 3.94655 | 8.86 |
| 23.7596 | 62.68 | 0.3464 | 3.74188 | 3.41 |
| 24.9664 | 73.15 | 0.2598 | 3.56367 | 3.98 |
| 27.0009 | 41.57 | 0.5196 | 3.29959 | 2.26 |
| 28.4320 | 96.61 | 0.2165 | 3.13668 | 5.26 |
| 31.3800 | 36.69 | 0.2598 | 2.84840 | 2.00 |
| 32.8030 | 37.98 | 0.2598 | 2.72801 | 2.07 |
| 35.8226 | 14.71 | 0.8659 | 2.50468 | 0.80 |

TABLE 10
XRPD Peak List of HCl salt Type D

| | | FWHM | | Rel. |
|---|---|---|---|---|
| Pos. [°2θ] | Height [cts] | Left [°2θ] | d-spacing [Å] | Int. [%] |
| 4.9355 | 618.24 | 0.1732 | 17.89003 | 72.13 |
| 6.8985 | 857.14 | 0.1948 | 12.80320 | 100.00 |
| 9.5387 | 195.60 | 0.1732 | 9.26456 | 22.82 |
| 10.9699 | 135.58 | 0.3031 | 8.05884 | 15.82 |
| 13.6789 | 68.28 | 0.2165 | 6.46835 | 7.97 |
| 15.3221 | 132.84 | 0.1299 | 5.77813 | 15.50 |
| 17.6832 | 134.21 | 0.2598 | 5.01161 | 15.66 |
| 18.4570 | 125.68 | 0.1299 | 4.80319 | 14.66 |
| 18.7804 | 81.01 | 0.1299 | 4.72120 | 9.45 |
| 19.3818 | 118.47 | 0.1299 | 4.57606 | 13.82 |
| 19.8124 | 228.35 | 0.1082 | 4.47756 | 26.64 |
| 20.9036 | 101.60 | 0.2165 | 4.24622 | 11.85 |
| 22.0215 | 261.36 | 0.1948 | 4.03312 | 30.49 |
| 22.6442 | 96.19 | 0.1299 | 3.92360 | 11.22 |
| 24.4737 | 101.32 | 0.2598 | 3.63429 | 11.82 |
| 25.0636 | 165.44 | 0.2165 | 3.55006 | 19.30 |
| 25.8848 | 170.33 | 0.1082 | 3.43927 | 19.87 |
| 28.0505 | 106.22 | 0.1299 | 3.17846 | 12.39 |
| 29.2018 | 109.13 | 0.1299 | 3.05572 | 12.73 |
| 29.8073 | 77.31 | 0.2598 | 2.99501 | 9.02 |
| 30.4360 | 62.16 | 0.2598 | 2.93455 | 7.25 |
| 31.5243 | 45.59 | 0.3464 | 2.83569 | 5.32 |
| 32.6439 | 63.65 | 0.2165 | 2.74094 | 7.43 |
| 35.1808 | 57.97 | 0.2598 | 2.54889 | 6.76 |
| 35.8001 | 58.17 | 0.2598 | 2.50620 | 6.79 |
| 36.6378 | 51.05 | 0.2598 | 2.45079 | 5.96 |
| 37.2661 | 42.92 | 0.2598 | 2.41091 | 5.01 |
| 38.9947 | 51.30 | 0.2598 | 2.30792 | 5.98 |

TABLE 11
XRPD Peak List of Compound A Sulfate

| | | FWHM | | Rel. |
|---|---|---|---|---|
| Pos. [°2θ] | Height [cts] | Left [°2θ] | d-spacing [Å] | Int. [%] |
| 3.4423 | 22.57 | 0.5196 | 25.64644 | 16.76 |
| 5.7071 | 134.66 | 0.1732 | 15.47314 | 100.00 |
| 6.9038 | 64.90 | 0.2598 | 12.79347 | 48.20 |
| 9.0376 | 53.32 | 0.3464 | 9.77709 | 39.59 |
| 16.8053 | 39.49 | 0.4330 | 5.27137 | 29.33 |
| 18.1556 | 129.13 | 0.1732 | 4.88226 | 95.89 |
| 20.0703 | 35.62 | 0.2598 | 4.42060 | 26.45 |
| 21.0051 | 48.34 | 0.2598 | 4.22592 | 35.90 |
| 24.6156 | 18.32 | 0.6061 | 3.61366 | 13.60 |

TABLE 12
XRPD Peak List of Compound A Napadisylate

| | | FWHM | | Rel. |
|---|---|---|---|---|
| Pos. [°2θ] | Height [cts] | Left [°2θ] | d-spacing [Å] | Int. [%] |
| 6.7421 | 489.07 | 0.1299 | 13.09991 | 71.05 |
| 8.0289 | 79.68 | 0.3464 | 11.00296 | 11.58 |
| 11.5606 | 201.02 | 0.2381 | 7.64837 | 29.20 |
| 14.6562 | 241.03 | 0.1082 | 6.03915 | 35.02 |
| 15.2654 | 239.53 | 0.1732 | 5.79949 | 34.80 |
| 15.5035 | 124.27 | 0.1082 | 5.71096 | 18.05 |
| 17.4722 | 485.37 | 0.1299 | 5.07164 | 70.51 |
| 18.5568 | 134.44 | 0.1299 | 4.77759 | 19.53 |
| 18.9113 | 51.82 | 0.1299 | 4.68883 | 7.53 |
| 19.3318 | 37.33 | 0.1299 | 4.58778 | 5.42 |
| 20.4293 | 216.24 | 0.1082 | 4.34372 | 31.41 |
| 20.7871 | 235.54 | 0.1299 | 4.26975 | 34.22 |
| 21.6993 | 41.48 | 0.2598 | 4.09227 | 6.03 |
| 22.1147 | 31.93 | 0.1515 | 4.01634 | 4.64 |
| 23.0184 | 170.28 | 0.1732 | 3.86066 | 24.74 |
| 23.4363 | 91.62 | 0.1299 | 3.79276 | 13.31 |
| 23.7627 | 148.09 | 0.2165 | 3.74139 | 21.51 |
| 24.3261 | 153.29 | 0.1299 | 3.65600 | 22.27 |
| 24.7879 | 688.35 | 0.1299 | 3.58893 | 100.00 |
| 26.6984 | 113.90 | 0.2165 | 3.33628 | 16.55 |
| 28.0158 | 38.94 | 0.2165 | 3.18232 | 5.66 |
| 28.5960 | 80.40 | 0.1732 | 3.11906 | 11.68 |
| 29.8694 | 129.94 | 0.1732 | 2.98892 | 18.88 |
| 30.4354 | 56.33 | 0.5196 | 2.93462 | 8.18 |
| 31.4037 | 64.39 | 0.1732 | 2.84630 | 9.35 |
| 32.3366 | 41.05 | 0.2598 | 2.76629 | 5.96 |
| 33.4158 | 42.80 | 0.2598 | 2.67937 | 6.22 |
| 34.8072 | 45.88 | 0.2165 | 2.57538 | 6.67 |
| 35.8458 | 29.82 | 0.2165 | 2.50311 | 4.33 |
| 36.9925 | 28.54 | 0.2598 | 2.42810 | 4.15 |
| 39.2623 | 5.98 | 0.5196 | 2.29280 | 0.87 |

TABLE 13
XRPD Peak List of Compound A Esylate

| Pos. | Height | FWHM Left | d-spacing | Rel. Int. |
|---|---|---|---|---|
| [°2θ] | [cts] | [°2θ] | [Å] | [%] |
| 5.9661 | 1853.17 | 0.1082 | 14.80191 | 100.00 |
| 10.2753 | 51.22 | 0.2165 | 8.60197 | 2.76 |
| 11.4808 | 238.41 | 0.1299 | 7.70133 | 12.86 |
| 13.3356 | 228.97 | 0.1082 | 6.63409 | 12.36 |
| 13.8716 | 69.29 | 0.1082 | 6.37890 | 3.74 |
| 15.0223 | 27.86 | 0.1299 | 5.89280 | 1.50 |
| 15.5509 | 72.58 | 0.1299 | 5.69365 | 3.92 |
| 17.4455 | 95.65 | 0.1299 | 5.07934 | 5.16 |
| 18.0754 | 114.89 | 0.1948 | 4.90372 | 6.20 |
| 18.5583 | 570.26 | 0.1732 | 4.77721 | 30.77 |
| 19.5270 | 605.54 | 0.1299 | 4.54234 | 32.68 |
| 20.8869 | 674.99 | 0.1299 | 4.24957 | 36.42 |
| 22.0658 | 258.01 | 0.1082 | 4.02512 | 13.92 |
| 22.8162 | 230.45 | 0.1299 | 3.89441 | 12.44 |
| 23.9256 | 207.10 | 0.1299 | 3.71628 | 11.18 |
| 24.6447 | 217.51 | 0.1082 | 3.60945 | 11.74 |
| 25.3802 | 515.85 | 0.1515 | 3.50649 | 27.84 |
| 26.0680 | 53.27 | 0.3464 | 3.41551 | 2.87 |
| 26.8112 | 106.78 | 0.1082 | 3.32250 | 5.76 |
| 27.4230 | 61.06 | 0.1299 | 3.24975 | 3.30 |
| 28.5136 | 105.75 | 0.1082 | 3.12789 | 5.71 |
| 29.0708 | 159.25 | 0.1299 | 3.06919 | 8.59 |
| 29.8150 | 112.52 | 0.1299 | 2.99425 | 6.07 |
| 30.4564 | 38.91 | 0.1299 | 2.93264 | 2.10 |
| 31.2013 | 74.95 | 0.1299 | 2.86430 | 4.04 |
| 32.1513 | 51.71 | 0.2598 | 2.78180 | 2.79 |
| 32.8428 | 50.26 | 0.1299 | 2.72480 | 2.71 |
| 34.0762 | 48.23 | 0.1299 | 2.62894 | 2.60 |
| 35.2358 | 50.79 | 0.1732 | 2.54503 | 2.74 |
| 37.0265 | 27.32 | 0.2598 | 2.42595 | 1.47 |
| 37.9577 | 47.21 | 0.3464 | 2.36855 | 2.55 |
| 38.6783 | 37.87 | 0.1732 | 2.32607 | 2.04 |

TABLE 14
XRPD Peak List of Compound A Mesylate

| Pos. | Height | FWHM Left | d-spacing | Rel. Int. |
|---|---|---|---|---|
| [°2θ] | [cts] | [°2θ] | [Å] | [%] |
| 3.2825 | 33.14 | 0.3464 | 26.89503 | 2.68 |
| 5.8087 | 1235.65 | 0.1515 | 15.20270 | 100.00 |
| 9.8567 | 44.54 | 0.1299 | 8.96634 | 3.60 |
| 11.3354 | 430.07 | 0.1515 | 7.79982 | 34.81 |
| 12.2781 | 23.06 | 0.2598 | 7.20300 | 1.87 |
| 14.0062 | 94.43 | 0.1515 | 6.31789 | 7.64 |
| 15.0546 | 93.19 | 0.1515 | 5.88021 | 7.54 |
| 16.3817 | 122.60 | 0.1082 | 5.40670 | 9.92 |
| 18.7240 | 101.31 | 0.1732 | 4.73530 | 8.20 |
| 19.1869 | 15.27 | 0.3031 | 4.62209 | 1.24 |
| 19.7902 | 352.44 | 0.1515 | 4.48252 | 28.52 |
| 20.3732 | 745.86 | 0.1515 | 4.35555 | 60.36 |
| 21.2953 | 78.26 | 0.1732 | 4.16900 | 6.33 |
| 21.7809 | 149.20 | 0.1082 | 4.07713 | 12.07 |
| 22.8449 | 191.75 | 0.1299 | 3.88959 | 15.52 |
| 23.2567 | 773.88 | 0.1515 | 3.82163 | 62.63 |
| 23.9960 | 84.80 | 0.1299 | 3.70553 | 6.86 |
| 25.1299 | 401.24 | 0.1515 | 3.54086 | 32.47 |
| 25.9030 | 127.98 | 0.1082 | 3.43691 | 10.36 |
| 26.5010 | 26.13 | 0.1732 | 3.36069 | 2.12 |
| 27.6267 | 101.33 | 0.1732 | 3.22625 | 8.20 |
| 28.2321 | 43.31 | 0.2165 | 3.15843 | 3.51 |
| 28.6932 | 87.72 | 0.1299 | 3.10871 | 7.10 |
| 29.0377 | 77.06 | 0.1299 | 3.07262 | 6.24 |
| 29.6785 | 145.40 | 0.2165 | 3.00771 | 11.77 |
| 30.0179 | 47.38 | 0.1515 | 2.97447 | 3.83 |
| 31.2648 | 43.60 | 0.3464 | 2.85863 | 3.53 |
| 33.2351 | 74.76 | 0.2165 | 2.69352 | 6.05 |

TABLE 15
XRPD Peak List of Compound A Napsylate

| Pos. | Height | FWHM Left | d-spacing | Rel. Int. |
|---|---|---|---|---|
| [°2θ] | [cts] | [°2θ] | [Å] | [%] |
| 6.5867 | 105.53 | 0.1299 | 13.40854 | 14.41 |
| 6.9597 | 139.87 | 0.1299 | 12.69071 | 19.10 |
| 8.9672 | 341.18 | 0.1732 | 9.85372 | 46.60 |
| 11.2783 | 85.85 | 0.1299 | 7.83918 | 11.73 |
| 11.9507 | 443.09 | 0.2598 | 7.39955 | 60.52 |
| 12.9574 | 266.02 | 0.1082 | 6.82687 | 36.34 |
| 13.3074 | 359.11 | 0.1299 | 6.64805 | 49.05 |
| 13.6039 | 119.05 | 0.1299 | 6.50384 | 16.26 |
| 14.0616 | 119.31 | 0.1732 | 6.29315 | 16.30 |
| 15.1976 | 594.54 | 0.1082 | 5.82521 | 81.21 |
| 16.3151 | 165.93 | 0.1732 | 5.42862 | 22.66 |
| 16.7982 | 156.59 | 0.1299 | 5.27357 | 21.39 |
| 17.1036 | 130.00 | 0.1082 | 5.18010 | 17.76 |
| 19.1097 | 257.16 | 0.1299 | 4.64058 | 35.12 |
| 19.7831 | 218.02 | 0.2165 | 4.48413 | 29.78 |
| 21.1287 | 257.28 | 0.1299 | 4.20148 | 35.14 |
| 22.0177 | 732.14 | 0.1299 | 4.03380 | 100.00 |
| 22.7960 | 151.05 | 0.1299 | 3.89782 | 20.63 |
| 23.6748 | 205.50 | 0.1082 | 3.75509 | 28.07 |
| 23.9529 | 164.92 | 0.3464 | 3.71211 | 22.53 |
| 24.2145 | 353.32 | 0.1082 | 3.67260 | 48.26 |
| 24.9292 | 437.43 | 0.1515 | 3.56890 | 59.75 |
| 25.6858 | 237.43 | 0.1515 | 3.46547 | 32.43 |
| 26.5422 | 110.35 | 0.1299 | 3.35556 | 15.07 |
| 26.8890 | 209.22 | 0.1515 | 3.31306 | 28.58 |
| 27.4205 | 305.33 | 0.1299 | 3.25004 | 41.70 |
| 28.7938 | 77.94 | 0.1732 | 3.09808 | 10.65 |
| 29.3745 | 146.44 | 0.1732 | 3.03814 | 20.00 |
| 30.3769 | 184.12 | 0.1515 | 2.94013 | 25.15 |
| 32.5353 | 51.43 | 0.2598 | 2.74984 | 7.03 |
| 33.4011 | 44.50 | 0.6927 | 2.68051 | 6.08 |
| 35.0398 | 52.39 | 0.1732 | 2.55882 | 7.16 |
| 36.4623 | 47.19 | 0.5196 | 2.46219 | 6.45 |
| 37.5593 | 56.75 | 0.2598 | 2.39276 | 7.75 |
| 38.3374 | 107.94 | 0.1299 | 2.34596 | 14.74 |
| 39.1127 | 54.87 | 0.2165 | 2.30123 | 7.49 |

TABLE 16
XRPD Peak List of Compound A Besylate

| Pos. | Height | FWHM Left | d-spacing | Rel. Int. |
|---|---|---|---|---|
| [°2θ] | [cts] | [°2θ] | [Å] | [%] |
| 3.9126 | 241.77 | 0.1082 | 22.56505 | 30.27 |
| 7.6967 | 33.66 | 0.5196 | 11.47717 | 4.21 |
| 11.7133 | 85.09 | 0.1732 | 7.54898 | 10.65 |
| 13.3459 | 144.94 | 0.1732 | 6.62896 | 18.15 |
| 13.8875 | 131.01 | 0.2598 | 6.37165 | 16.40 |
| 14.7828 | 128.23 | 0.1732 | 5.98772 | 16.05 |
| 15.4841 | 214.34 | 0.1732 | 5.71805 | 26.84 |
| 16.3202 | 130.38 | 0.2598 | 5.42694 | 16.32 |
| 19.1489 | 798.72 | 0.1082 | 4.63118 | 100.00 |
| 19.6852 | 414.44 | 0.3031 | 4.50621 | 51.89 |
| 21.9112 | 280.12 | 0.2598 | 4.05318 | 35.07 |
| 24.9182 | 771.18 | 0.1082 | 3.57045 | 96.55 |
| 26.0393 | 262.64 | 0.3031 | 3.41922 | 32.88 |
| 26.8954 | 192.18 | 0.2598 | 3.31229 | 24.06 |
| 27.6178 | 153.44 | 0.2598 | 3.22727 | 19.21 |
| 29.8350 | 96.98 | 0.5196 | 2.99229 | 12.14 |
| 32.4503 | 80.95 | 0.2598 | 2.75685 | 10.14 |
| 32.9975 | 74.59 | 0.2598 | 2.71238 | 9.34 |
| 35.5952 | 52.20 | 0.2598 | 2.52016 | 6.54 |
| 37.3617 | 29.01 | 0.5196 | 2.40496 | 3.63 |

TABLE 17
XRPD Peak List of Compound A Sodium salt

| Pos. | Height | FWHM Left | d-spacing | Rel. Int. |
|---|---|---|---|---|
| [°2θ] | [cts] | [°2θ] | [Å] | [%] |
| 3.2419 | 106.53 | 0.1732 | 27.23116 | 50.24 |
| 9.7412 | 141.11 | 0.3464 | 9.07246 | 66.56 |
| 13.1852 | 12.74 | 0.5196 | 6.70939 | 6.01 |
| 14.6212 | 35.30 | 0.3464 | 6.05351 | 16.65 |
| 16.2219 | 51.16 | 0.2598 | 5.45961 | 24.13 |
| 18.1390 | 14.13 | 0.8659 | 4.88667 | 6.67 |
| 23.5691 | 212.02 | 0.3031 | 3.77168 | 100.00 |

TABLE 18
XRPD Peak List of Compound A Potassium salt

| Pos. | Height | FWHM Left | d-spacing | Rel. Int. |
|---|---|---|---|---|
| [°2θ] | [cts] | [°2θ] | [Å] | [%] |
| 6.2264 | 70.30 | 0.1299 | 14.18374 | 38.80 |
| 9.4071 | 181.21 | 0.1299 | 9.39390 | 100.00 |
| 12.5888 | 29.90 | 0.2598 | 7.02591 | 16.50 |
| 15.8704 | 58.41 | 0.1732 | 5.57972 | 32.23 |
| 19.0223 | 38.58 | 0.2598 | 4.66172 | 21.29 |
| 23.9796 | 143.84 | 0.1732 | 3.70804 | 79.37 |
| 37.7334 | 14.11 | 0.2598 | 2.38212 | 7.78 |

TABLE 19
XRPD Peak List of Compound A L-Arginine salt

| Pos. | Height | FWHM Left | d-spacing | Rel. Int. |
|---|---|---|---|---|
| [°2θ] | [cts] | [°2θ] | [Å] | [%] |
| 3.2187 | 33.28 | 0.3464 | 27.42729 | 4.66 |
| 6.3355 | 714.20 | 0.1515 | 13.93966 | 100.00 |
| 9.2720 | 198.90 | 0.1515 | 9.53043 | 27.85 |
| 10.6093 | 51.67 | 0.1299 | 8.33191 | 7.23 |
| 12.6548 | 260.65 | 0.1732 | 6.98940 | 36.50 |
| 15.1522 | 90.68 | 0.1082 | 5.84254 | 12.70 |
| 16.1408 | 56.93 | 0.1299 | 5.48687 | 7.97 |
| 18.6404 | 207.11 | 0.2598 | 4.75636 | 29.00 |
| 19.0901 | 610.04 | 0.1948 | 4.64531 | 85.42 |
| 19.5670 | 684.30 | 0.1732 | 4.53314 | 95.81 |
| 20.4160 | 88.28 | 0.2165 | 4.34652 | 12.36 |
| 20.9892 | 243.88 | 0.1515 | 4.22909 | 34.15 |
| 21.4733 | 139.89 | 0.1732 | 4.13482 | 19.59 |
| 22.0475 | 269.25 | 0.1732 | 4.02843 | 37.70 |
| 22.7612 | 489.25 | 0.1732 | 3.90370 | 68.50 |
| 23.8751 | 346.57 | 0.1732 | 3.72404 | 48.53 |
| 24.0774 | 239.81 | 0.1299 | 3.69319 | 33.58 |
| 24.8633 | 135.16 | 0.1082 | 3.57821 | 18.92 |
| 25.7119 | 121.42 | 0.2165 | 3.46201 | 17.00 |
| 26.3948 | 83.28 | 0.3464 | 3.37396 | 11.66 |
| 27.2669 | 147.54 | 0.1732 | 3.26800 | 20.66 |
| 27.5789 | 140.34 | 0.1299 | 3.23174 | 19.65 |
| 28.0796 | 109.40 | 0.1299 | 3.17524 | 15.32 |
| 29.8400 | 109.56 | 0.1732 | 2.99180 | 15.34 |
| 30.3077 | 122.18 | 0.2598 | 2.94669 | 17.11 |
| 30.7501 | 88.78 | 0.1732 | 2.90529 | 12.43 |
| 31.4016 | 70.97 | 0.2165 | 2.84649 | 9.94 |
| 32.5990 | 53.40 | 0.1732 | 2.74461 | 7.48 |
| 33.4990 | 65.36 | 0.1732 | 2.67290 | 9.15 |
| 34.8636 | 73.76 | 0.2598 | 2.57134 | 10.33 |
| 36.5905 | 61.42 | 0.4330 | 2.45386 | 8.60 |

TABLE 20
XRPD Peak List of Compound A Choline salt

| Pos. | Height | FWHM Left | d-spacing | Rel. Int. |
|---|---|---|---|---|
| [°2θ] | [cts] | [°2θ] | [Å] | [%] |
| 5.6014 | 59.28 | 0.1732 | 15.76482 | 14.08 |
| 8.4336 | 261.74 | 0.2165 | 10.47593 | 62.16 |
| 11.3521 | 421.05 | 0.1948 | 7.78835 | 100.00 |
| 14.3597 | 45.23 | 0.5196 | 6.16319 | 10.74 |
| 15.3296 | 34.53 | 0.3464 | 5.77535 | 8.20 |
| 17.0869 | 348.31 | 0.2381 | 5.18513 | 82.72 |
| 20.2730 | 23.49 | 0.8659 | 4.37686 | 5.58 |
| 23.9518 | 238.86 | 0.2598 | 3.71228 | 56.73 |
| 25.9191 | 106.88 | 0.1515 | 3.43480 | 25.39 |
| 28.7655 | 62.02 | 0.2598 | 3.10107 | 14.73 |

TABLE 21
XRPD Peak List of Compound A L-Lysine salt

| Pos. | Height | FWHM Left | d-spacing | Rel. Int. |
|---|---|---|---|---|
| [°2θ] | [cts] | [°2θ] | [Å] | [%] |
| 5.2335 | 73.38 | 0.2165 | 16.87226 | 23.17 |
| 7.9040 | 316.66 | 0.1732 | 11.17662 | 100.00 |
| 8.6800 | 93.16 | 0.4330 | 10.17905 | 29.42 |
| 12.2312 | 43.39 | 0.2598 | 7.23050 | 13.70 |
| 13.6072 | 34.02 | 0.3464 | 6.50226 | 10.74 |
| 15.3535 | 37.70 | 0.8659 | 5.76642 | 11.91 |
| 17.3105 | 221.07 | 0.2165 | 5.11865 | 69.81 |
| 20.0993 | 100.51 | 0.2598 | 4.41427 | 31.74 |
| 21.7705 | 206.43 | 0.3464 | 4.07905 | 65.19 |
| 22.5832 | 115.50 | 0.2598 | 3.93406 | 36.47 |
| 24.0319 | 154.76 | 0.2165 | 3.70009 | 48.87 |
| 25.1203 | 68.50 | 0.2598 | 3.54218 | 21.63 |
| 27.4979 | 48.10 | 0.2598 | 3.24108 | 15.19 |
| 30.9095 | 22.28 | 0.6061 | 2.89068 | 7.04 |
| 31.7091 | 20.93 | 0.2598 | 2.81958 | 6.61 |
| 32.9338 | 18.48 | 0.4330 | 2.71747 | 5.84 |
| 35.3235 | 28.64 | 0.5196 | 2.53892 | 9.04 |

TABLE 22
XRPD Peak List of Compound A t-Butylamine salt

| Pos. | Height | FWHM Left | d-spacing | Rel. Int. |
|---|---|---|---|---|
| [°2θ] | [cts] | [°2θ] | [Å] | [%] |
| 6.3785 | 878.13 | 0.1082 | 13.84573 | 31.95 |
| 6.6377 | 549.77 | 0.1082 | 13.30565 | 20.00 |
| 8.6274 | 158.15 | 0.1082 | 10.24095 | 5.75 |
| 10.9907 | 197.31 | 0.1082 | 8.04364 | 7.18 |
| 11.4472 | 2748.18 | 0.1299 | 7.72390 | 100.00 |
| 13.3938 | 676.07 | 0.1299 | 6.60536 | 24.60 |
| 14.4889 | 61.47 | 0.2165 | 6.10850 | 2.24 |
| 14.9504 | 405.52 | 0.1082 | 5.92094 | 14.76 |
| 15.3885 | 71.56 | 0.1299 | 5.75337 | 2.60 |
| 16.4631 | 50.58 | 0.1299 | 5.38017 | 1.84 |
| 16.9462 | 121.86 | 0.1299 | 5.22786 | 4.43 |
| 17.6747 | 309.39 | 0.1082 | 5.01399 | 11.26 |
| 17.9371 | 437.94 | 0.1082 | 4.94123 | 15.94 |
| 19.4661 | 266.07 | 0.1299 | 4.55642 | 9.68 |
| 20.1917 | 191.86 | 0.1082 | 4.39429 | 6.98 |
| 20.9739 | 557.62 | 0.1299 | 4.23213 | 20.29 |
| 21.4411 | 537.24 | 0.1299 | 4.14097 | 19.55 |
| 22.0596 | 402.35 | 0.1082 | 4.02624 | 14.64 |
| 22.8266 | 118.08 | 0.1082 | 3.89266 | 4.30 |
| 23.2928 | 146.39 | 0.1299 | 3.81580 | 5.33 |
| 23.7463 | 103.42 | 0.1082 | 3.74393 | 3.76 |
| 24.0794 | 145.82 | 0.1082 | 3.69290 | 5.31 |
| 24.6680 | 177.16 | 0.1082 | 3.60610 | 6.45 |
| 26.0985 | 441.06 | 0.1082 | 3.41160 | 16.05 |
| 27.0961 | 175.34 | 0.1732 | 3.28822 | 6.38 |
| 27.7333 | 104.09 | 0.1732 | 3.21410 | 3.79 |
| 28.1778 | 80.10 | 0.2165 | 3.16439 | 2.91 |
| 29.0755 | 117.67 | 0.1299 | 3.06870 | 4.28 |
| 29.7597 | 38.49 | 0.2165 | 2.99969 | 1.40 |
| 30.6363 | 63.29 | 0.1299 | 2.91582 | 2.30 |
| 31.1206 | 114.19 | 0.1299 | 2.87155 | 4.16 |
| 31.4896 | 49.38 | 0.3031 | 2.83873 | 1.80 |
| 32.1595 | 45.84 | 0.1732 | 2.78111 | 1.67 |
| 32.6294 | 109.24 | 0.1082 | 2.74212 | 3.98 |
| 34.5749 | 80.00 | 0.2598 | 2.59215 | 2.91 |
| 35.0565 | 108.78 | 0.1082 | 2.55763 | 3.96 |
| 35.8441 | 32.91 | 0.3464 | 2.50323 | 1.20 |
| 37.0836 | 18.50 | 0.5196 | 2.42235 | 0.67 |

TABLE 23
XRPD Peak List of Compound A Meglumine salt

| Pos. | Height | FWHM Left | d-spacing | Rel. Int. |
|---|---|---|---|---|
| [°2θ] | [cts] | [°2θ] | [Å] | [%] |
| 3.4397 | 18.93 | 0.5196 | 25.66597 | 3.47 |
| 5.5733 | 64.59 | 0.2165 | 15.84418 | 11.83 |
| 8.7694 | 140.79 | 0.1299 | 10.07547 | 25.79 |
| 11.3408 | 79.28 | 0.2165 | 7.79607 | 14.52 |
| 14.0687 | 114.38 | 0.1299 | 6.28997 | 20.95 |
| 17.1061 | 73.82 | 0.1732 | 5.17936 | 13.52 |
| 20.5440 | 29.74 | 0.8659 | 4.31972 | 5.45 |
| 22.0407 | 28.01 | 0.5196 | 4.02965 | 5.13 |
| 23.9326 | 545.91 | 0.1732 | 3.71522 | 100.00 |
| 26.5102 | 63.01 | 0.5196 | 3.35954 | 11.54 |
| 28.7996 | 20.97 | 0.6927 | 3.09747 | 3.84 |

TABLE 24
XRPD Peak List of Compound A Tris salt

| Pos. | Height | FWHM Left | d-spacing | Rel. Int. |
|---|---|---|---|---|
| [°2θ] | [cts] | [°2θ] | [Å] | [%] |
| 3.7388 | 299.49 | 0.1082 | 23.61331 | 20.62 |
| 7.4812 | 1452.72 | 0.1732 | 11.80726 | 100.00 |
| 9.4801 | 80.11 | 0.1299 | 9.32172 | 5.51 |
| 10.3096 | 93.30 | 0.2165 | 8.57344 | 6.42 |
| 11.2295 | 201.01 | 0.1299 | 7.87315 | 13.84 |
| 12.7589 | 91.84 | 0.1732 | 6.93262 | 6.32 |
| 14.9451 | 210.09 | 0.1082 | 5.92304 | 14.46 |
| 17.8358 | 217.23 | 0.1082 | 4.96906 | 14.95 |
| 18.4138 | 160.93 | 0.1515 | 4.81437 | 11.08 |
| 18.7503 | 448.79 | 0.1515 | 4.72871 | 30.89 |
| 19.0330 | 1002.52 | 0.1299 | 4.65912 | 69.01 |
| 19.6588 | 330.18 | 0.1299 | 4.51219 | 22.73 |
| 20.6145 | 332.27 | 0.1082 | 4.30512 | 22.87 |
| 21.5454 | 404.73 | 0.1515 | 4.12116 | 27.86 |
| 22.4384 | 623.83 | 0.1299 | 3.95912 | 42.94 |
| 22.6192 | 573.38 | 0.1299 | 3.92788 | 39.47 |
| 23.7369 | 108.79 | 0.1515 | 3.74541 | 7.49 |
| 25.0243 | 311.41 | 0.1732 | 3.55555 | 21.44 |
| 25.7484 | 248.04 | 0.1515 | 3.45718 | 17.07 |
| 26.3942 | 254.03 | 0.2165 | 3.37404 | 17.49 |
| 27.3205 | 83.04 | 0.1299 | 3.26171 | 5.72 |
| 27.7745 | 373.07 | 0.1948 | 3.20942 | 25.68 |
| 28.7102 | 227.16 | 0.1299 | 3.10692 | 15.64 |
| 29.1715 | 182.72 | 0.1082 | 3.05882 | 12.58 |
| 29.3498 | 217.66 | 0.1299 | 3.04064 | 14.98 |
| 30.8184 | 54.13 | 0.3464 | 2.89902 | 3.73 |
| 31.1283 | 101.86 | 0.1299 | 2.87085 | 7.01 |
| 31.5537 | 113.41 | 0.1299 | 2.83311 | 7.81 |
| 31.8537 | 99.93 | 0.1732 | 2.80712 | 6.88 |
| 32.4757 | 62.46 | 0.1732 | 2.75476 | 4.30 |
| 33.9376 | 31.41 | 0.2598 | 2.63936 | 2.16 |
| 34.4828 | 52.46 | 0.2165 | 2.59887 | 3.61 |
| 35.3983 | 76.09 | 0.5196 | 2.53372 | 5.24 |
| 37.2963 | 79.16 | 0.2598 | 2.40903 | 5.45 |
| 38.6118 | 42.03 | 0.1299 | 2.32992 | 2.89 |
| 38.9886 | 59.41 | 0.2165 | 2.30827 | 4.09 |

### 5.1.3. HCl Studies

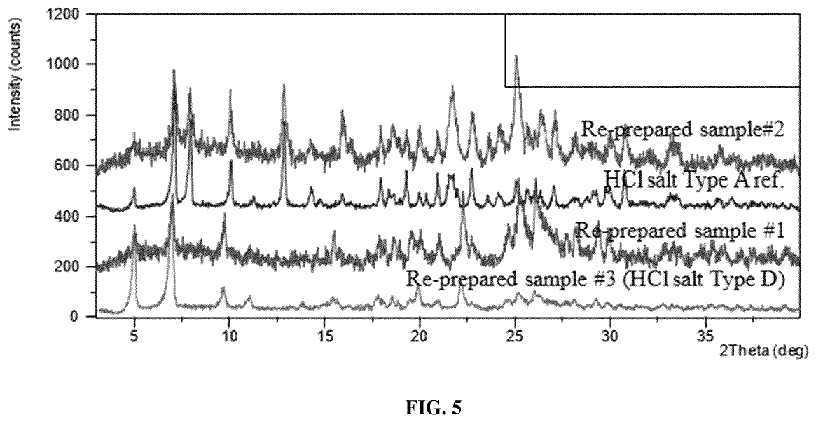

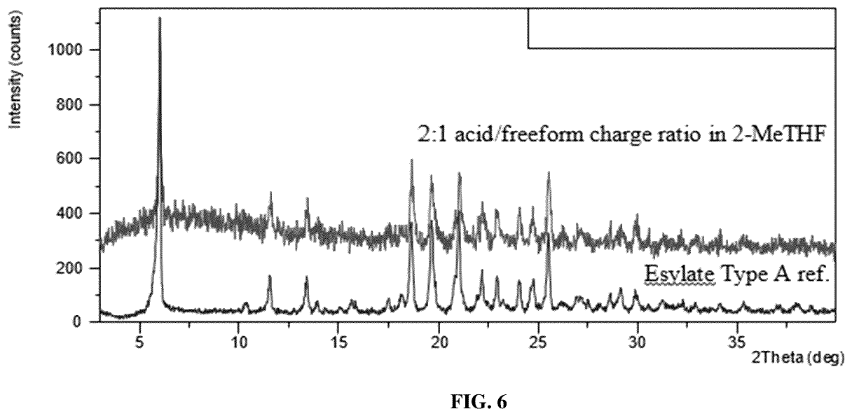

As illustrated in Table 4, HCl salt Type B demonstrated good solid-state properties; therefore, the isolation of HCl salt Type B was attempted in methyl ethyl ketone (MEK) in triplicate using HCl salt Type B as seeds. Previously, only the mono-HCl salt was obtained in the salt experiments with a charge ratio of 1:1 and 2:1 (HCl/freeform), therefore the isolation of HCl salt Type B was attempted with a charge molar ratio of 1:1. The details and results were summarized in Table 25 and the XRPD diffractogram was shown in FIG. 5. A gel was obtained after adding acids, and therefore failed to obtain HCl salt Type B after being transferred to a temperature cycling slurry. A new form was obtained, identified as HCl salt Type D. Due to the poor reproducibility of HCl salt Type B and relatively complicated thermal events of the other three HCl salts, the HCl salt of Compound A was not chosen for further study.

TABLE 25
Summary of HCl salt Type B study

| | Charge | | | | |
|---|---|---|---|---|---|
| | molar | | | | |
| Mass of | ratio | | | | |
| freeform | (acid: | | Volume | | |
| (mg) | freeform) | Solvents | (mL) | Method* | Results |
| 100 | 1:1 | MEK | 1 | Gel after | HCl salt |
| | | | | adding acid, | Type D |
| | | | | transfer to | |
| | | | | temperature | |
| | | | | cycling | |
| | | | | slurry | |
| 100 | 1:1 | | 1 | Gel after | HCl salt |
| | | | | adding acid, | Type A |
| | | | | transfer to | |
| | | | | temperature | |
| | | | | cycling | |
| | | | | slurry | |
| 50 | 1:1 | | 0.5 | Gel after | HCl salt |
| | | | | adding acid, | Type D |
| | | | | transfer to | |
| | | | | temperature | |
| | | | | cycling | |
| | | | | slurry | |

*Temperature cycling slurry: 50° C.-5° C.-50° C. 0.1° C./min

### 5.1.4. Stress Test of Compound A Esylate Type A

Compound A Esylate Type A was found to exhibit good solid-state properties therefore was selected for further evaluation. A stress test of the esylate salt was performed with different charge ratios to investigate the possibility of di-salt formation since the freeform was dibasic and ethanesulfonic acid was a strong acid.

TABLE 26
Stress Test of Compound A esylate salt

| Charge ratio | | | |
|---|---|---|---|
| (acid:freeform) | Solvents | Method* | Results |
| 2:1 | MEK | Temperature cycling slurry | Gel |
| 0.5:1 | | Slurry at 5° C. | Clear solution |
| 2:1 | EtOAc | Temperature cycling slurry | Gel |
| 2:1 | 2-MeTHF | Temperature cycling slurry | Esylate Type A |

*Temperature cycling slurry: 50° C.-5° C.-50° C. 0.1° C./min

Based on the solid-state properties and reproducibility of the esylate salt, the t-butylamine salt, and the tris salt of Compound A, these salts were further evaluated and compared to Compound A freeform Type B. The preparation of each is provided in Table 27.

TABLE 27
Preparation of Solid forms of Compound
Freeform Type B and Salts of Compound A

| Salt | Procedure | |
|---|---|---|
| Freeform | 1. | A 20-mL vial was charged with 397.85 mg of amorphous |
| Type B | | freeform and seeded with freeform Type B. |
| | 2. | 5 mL of EtOAc was gradually added into the solids to |
| | | form a suspension. |
| | 3. | The suspension was magnetically stirred at 400 rpm at |
| | | r.t. for 1 day. |
| | 4. | The solids were isolated by vacuum filtration and |
| | | vacuum dried at 40° C. overnight. |
| | 5. | 240.82 mg of solids was collected in 60.5% yield. |
| Esylate | 1. | 400.84 mg of amorphous freeform was added into 3 mL |
| Type A | | of 2-MeTHF to obtain a clear freeform solution. |
| | 2. | 183.32 mg of ethanesulfonic acid was dissolved in |
| | | 6 mL of 2-MeTHF to obtain a clear acid solution. |
| | 3. | 3 mL of the acid solution from step 2 was slowly |
| | | added into the freeform solution from step 1 (charge |
| | | ratio of 1:1) with Compound A esylate Type A crystal |
| | | seeds. |
| | 4. | The suspension was magnetically stirred at 400 rpm |
| | | along with temperature cycling (50° C.-5° C.-50° C., |
| | | 0.1° C./min) for 1 day. |
| | 5. | The solids were isolated by vacuum filtration and |
| | | vacuum dry at 40° C. overnight. |
| | 6. | 434.60 mg of solids was collected in 88.3% yield. |
| t-Butylamine | 1. | 400.91 mg of amorphous freeform was added into 3 mL |
| salt | | of EtOAc to obtain a clear freeform solution. |
| Type A | 2. | 175 μL of t-butylamine was dissolved in 6 mL of |
| | | EtOAc to obtain a clear base solution. |
| | 3. | 3 mL of the base solution from step 2 was slowly |
| | | added into the freeform solution of step 1 (charge ratio |
| | | of 1:1) with Compound A t-butylamine salt Type A crystal |
| | | seeds. |
| | 4. | The solution was magnetically stirred at 400 rpm at |
| | | r.t for 1 day. |
| | 5. | The formed solids were isolated by vacuum filtration |
| | | and vacuum dried at 40° C. for 7 h. |
| | 6. | 309.63 mg of solids was collected in 67.0% yield. |
| Tris salt | 1. | A 20-mL vial was charged with 400.72 mg of amorphous |
| Type A | | freeform and 100.81 mg of tromethamine (charge ratio |
| | | of 1:1). |
| | 2. | 5 mL of MEK was gradually added into the solids to |
| | | form a suspension with tris salt Type A crystal seeds. |

### 5.1.5. Compound a Freeform Type B

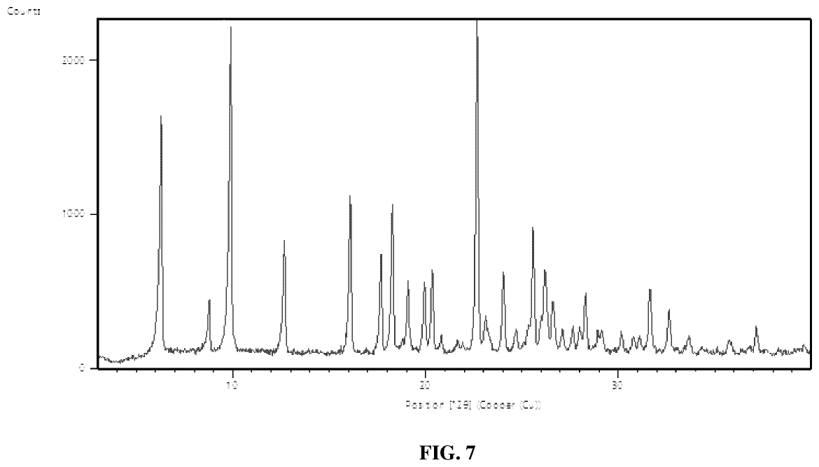

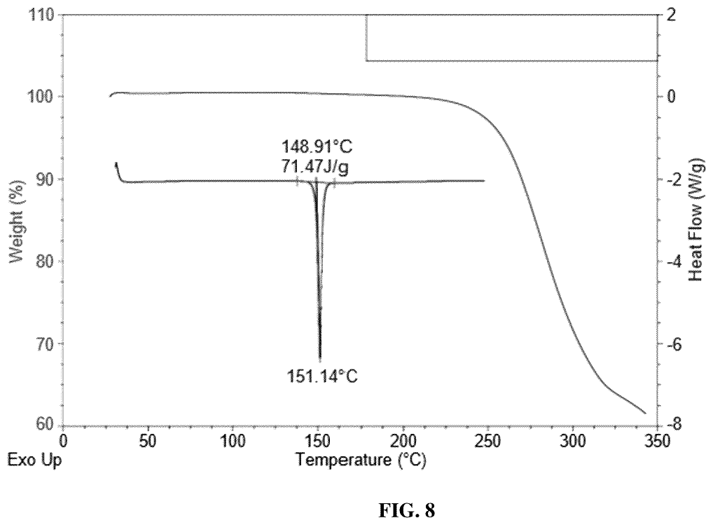

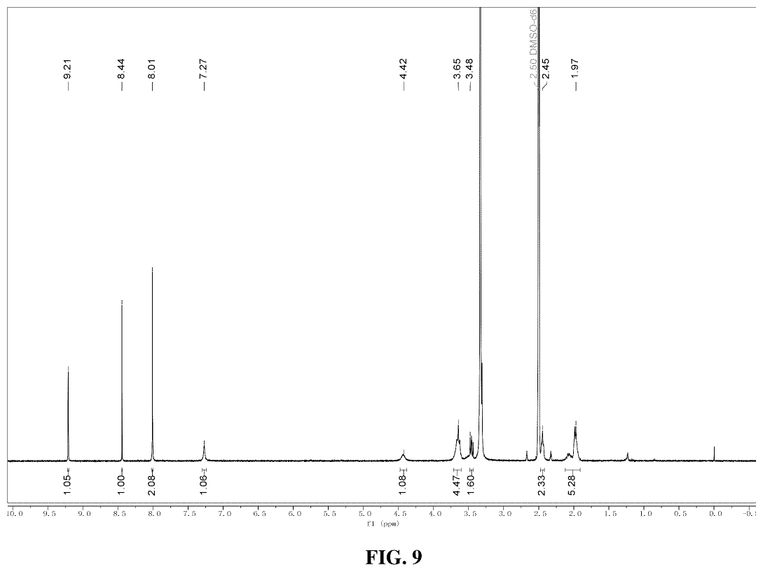

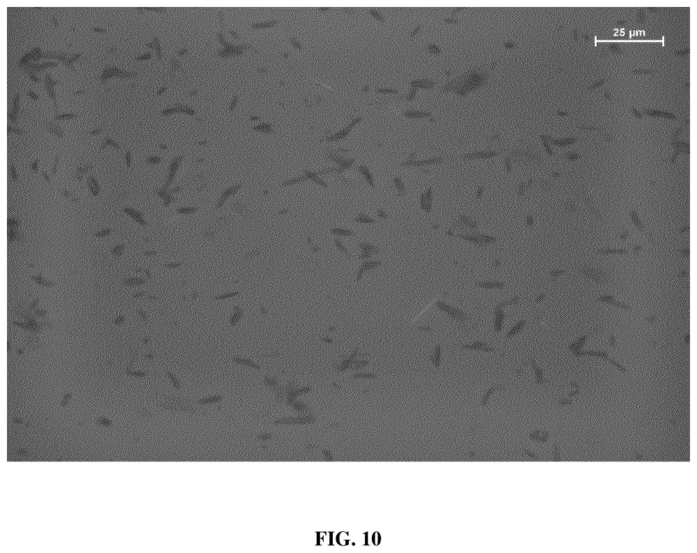

Compound A freeform Type B was isolated using EtOAc. The XRPD diffractogram is shown in FIG. 7, indicating the obtained solids were freeform Type B. The TGA result (FIG. 8) showed negligible weight loss before decomposition, and the DSC result showed one endothermic peak at 148.9° C. (onset). ¹H NMR spectrum (FIG. 9) showed no residual EtOAc. The PLM image (FIG. 10) showed the sample was consisted of rod-like crystals. The HPLC purity was 99.06 area %. ¹HNMR (400 MHz, DMSO-d₆): 9.21 (s, 1H), 8.44 (s, 1H), 8.01 (s, 2H), 7.21 (s, 1H), 4.42 (bs, 1H), 3.65 (m, 4H), 3.48 (m, 2H), 2.50-2.45 (m, 2H), 1.97 (m, 4H) ppm.

5.1.6. Compound A esylate Type A

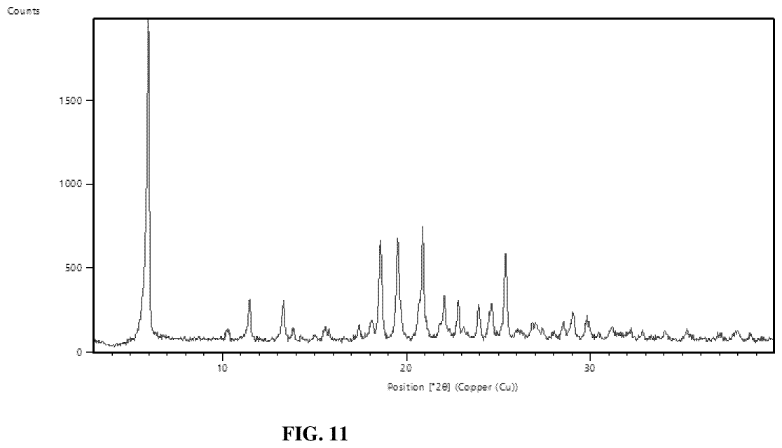

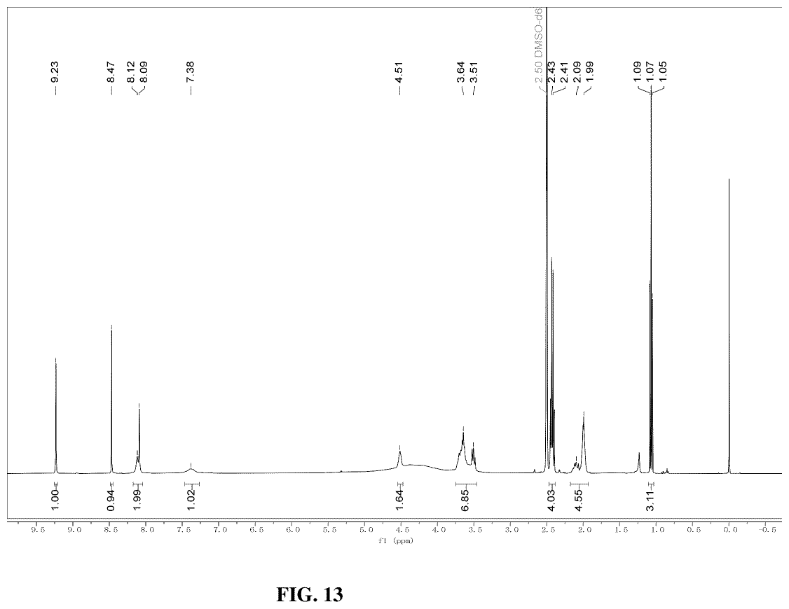

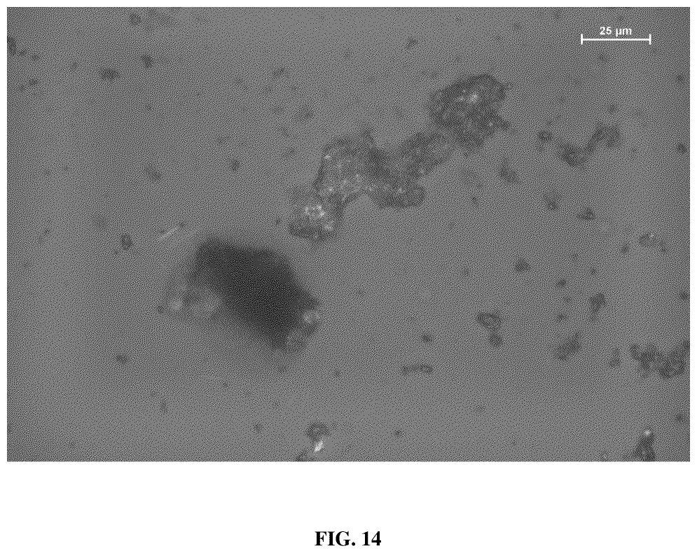

Compound A esylate Type A was isolated using 2-MeTHF. The XRPD diffractogram is shown in FIG. 11, indicating the obtained solids were esylate Type A. The TGA result (FIG. 12) showed negligible weight loss before decomposition, and the DSC result showed one endothermic peak at 208.3° C. (onset). ¹H NMR spectrum of the isolated esylate Type A (FIG. 13) showed the stoichiometry of ethanesulfonic acid/freeform was 1.0 with no residual MEK. The PLM image (FIG. 14) showed the sample consisted of agglomerated crystals. The HPLC purity was 97.28 area %. ¹HNMR (400 MHz, DMSO-d₆): 9.23 (s, 1H), 8.47 (s, 1H), 8.12-8.09 (m, 2H), 7.38 (bs, 1H), 4.51 (bs, 1H), 3.64-3.51 (m, 6H), 2.43-2.41 (m, 4H), 2.09-1.99 (m, 4H), 1.09-1.05 (t, J=8 Hz, 3H) ppm.

5.1.7. Compound A t-butylamine Salt Type A

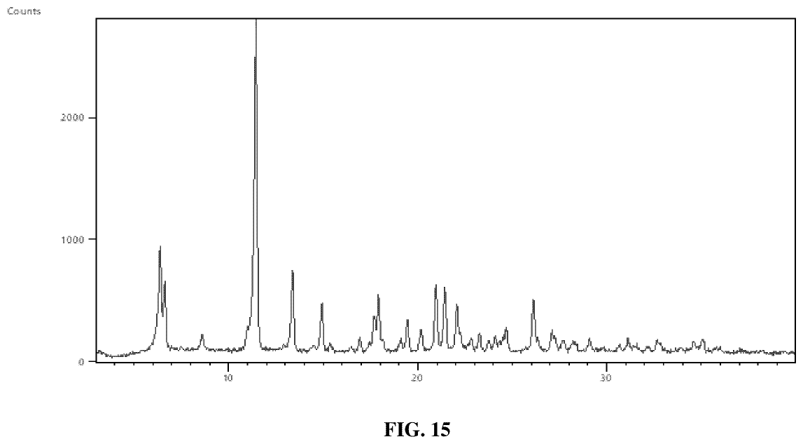

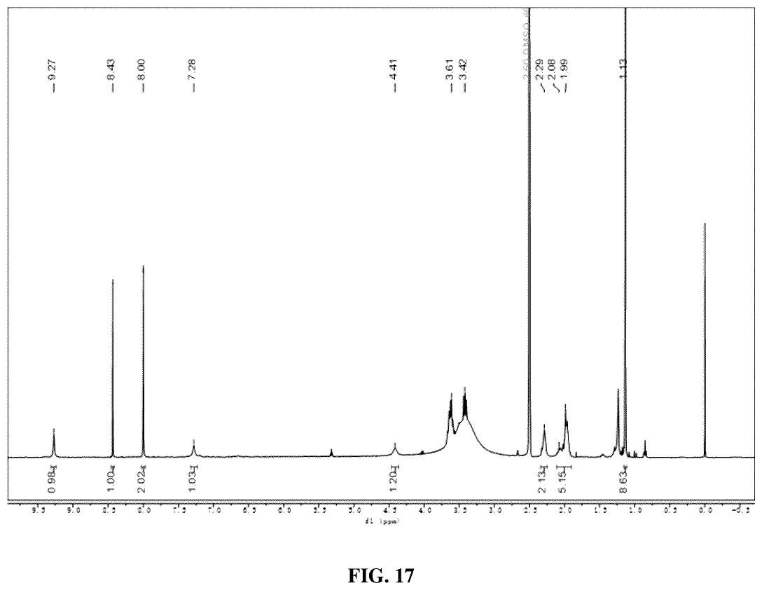

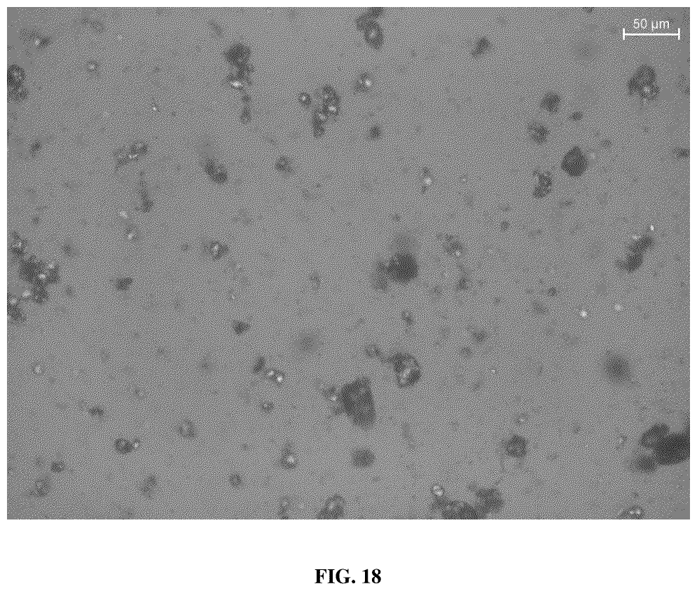

Compound A t-butylamine salt Type A was isolated using EtOAc. The XRPD diffractograms were shown in FIG. 15, indicating the obtained solids were t-butylamine salt Type A. The TGA result (FIG. 16) showed negligible weight loss before 100° C. followed by a weight loss of 13.1% from 100 to 210° C. (13.2% for 1 eq. of t-butylamine), and the DSC result showed one endothermic peak at 166.7° C. (onset). ¹H NMR spectrum of the isolated t-butylamine salt Type A (FIG. 17) showed the stoichiometry of t-butylamine/freeform was 1.0 with trace EtOAc (˜0.03%). The PLM image (FIG. 18) showed the sample was consisted of small crystals. The HPLC purity was 98.04 area %. ¹HNMR (400 MHz, DMSO-d₆): 9.27 (s, 1H), 8.43 (s, 1H), 8.00 (s, 2H), 7.28 (bs, 1H), 4.41 (bs, 1H), 3.61 (m, 4H), 3.42 (m, 2H), 2.29 (m, 2H), 2.08-1.99 (m, 4H), 1.13 (s, 9H) ppm.

### 5.1.8. Compound a Tris Salt Type A

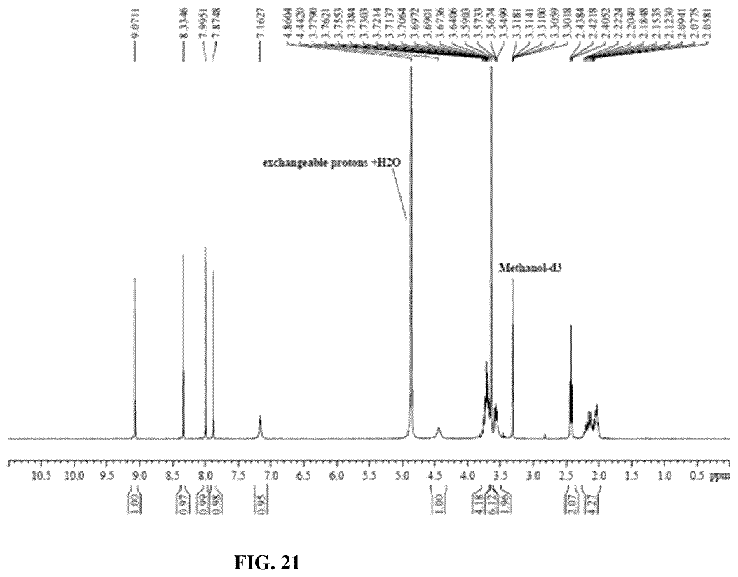

Compound A tris salt type A was isolated using MEK. The XRPD diffractograms, shown in FIG. 19, indicating the obtained solids were tris salt Type A. The TGA result (FIG. 20) showed negligible weight loss before decomposition, and the DSC result showed one endothermic peak at 159.8° C. (onset). ¹H NMR spectrum of the isolated tris salt Type A (FIG. 21) showed the stoichiometry of tromethamine/freeform was 1.0 with no residual MEK. PLM image (FIG. 22) showed the sample was consisted of small crystals. The HPLC was 97.43% area. ¹HNMR (400 MHz, CD₃OD): 9.07 (s, 1H), 8.33 (s, 1H), 8.00 (s, 1H), 7.87 (s, 1H), 7.16 (s, 1H), 4.44 (bs, 1H), 3.78-3.67 (m, 4H), 3.64 (s, 6H), 3.59-3.55 (m, 2H), 2.42 (t, J=6.6 Hz, 2H), 2.22-1.99 (m, 4H).

### 5.2. Evaluation of Salts

The isolated solid forms of Compound A esylate, Compound A t-butylamine salt, and Compound A tris salt were further evaluated on hygroscopicity, kinetic solubility, and physicochemical stability, using freeform Type B as a control.

### 5.2.1. Hygroscopicity

To evaluate the hygroscopicity of the esylate, t-butylamine salt, tris salt, and freeform Type B, DVS was employed to measure the mass change as a function of relative humidity at 25° C. All samples were pre-dried at 0% RH to remove adsorbed moisture or solvent.

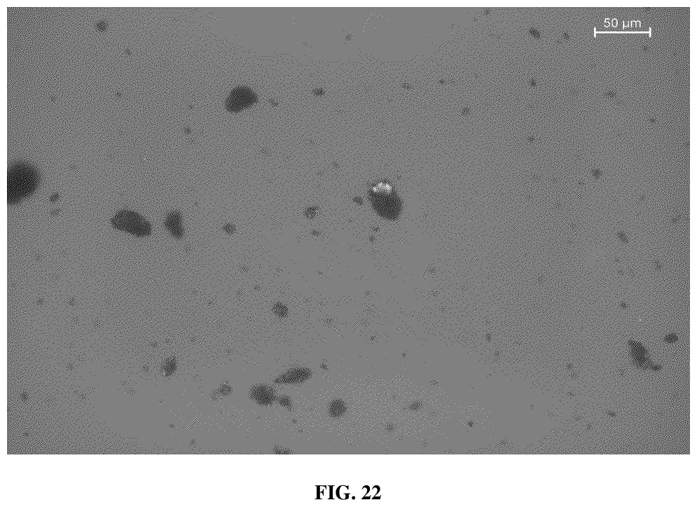

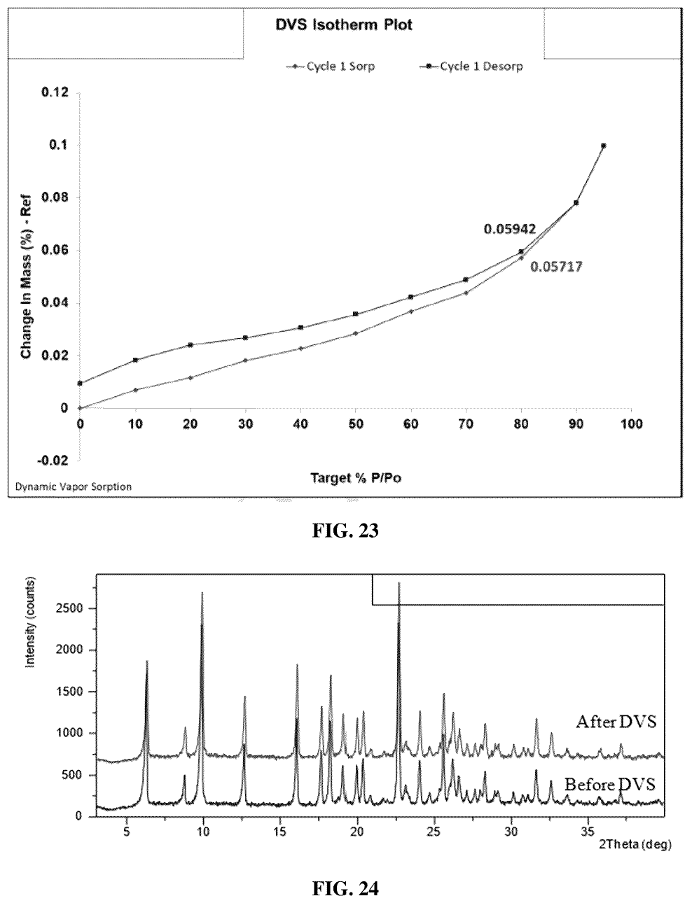

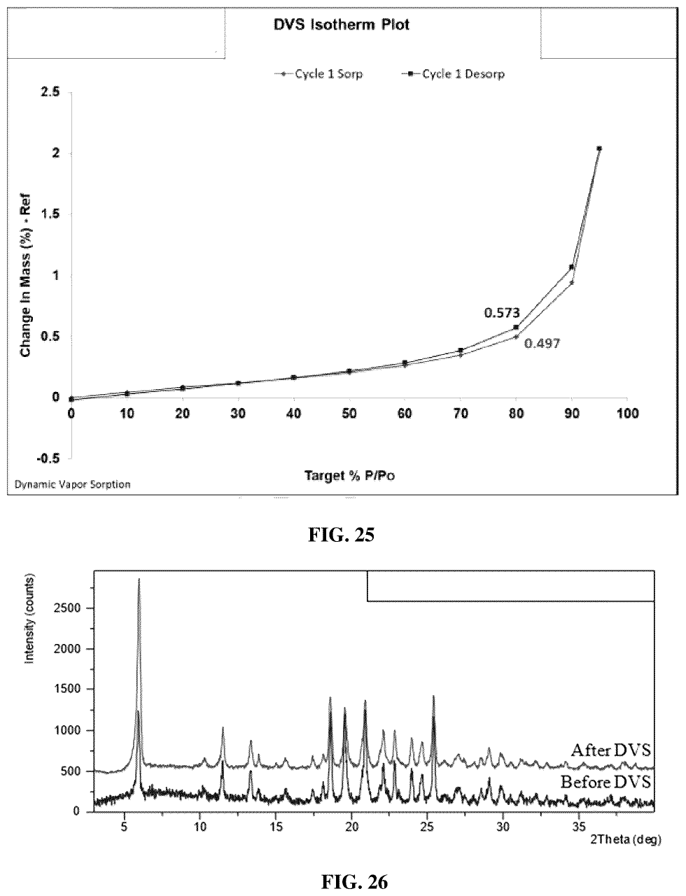

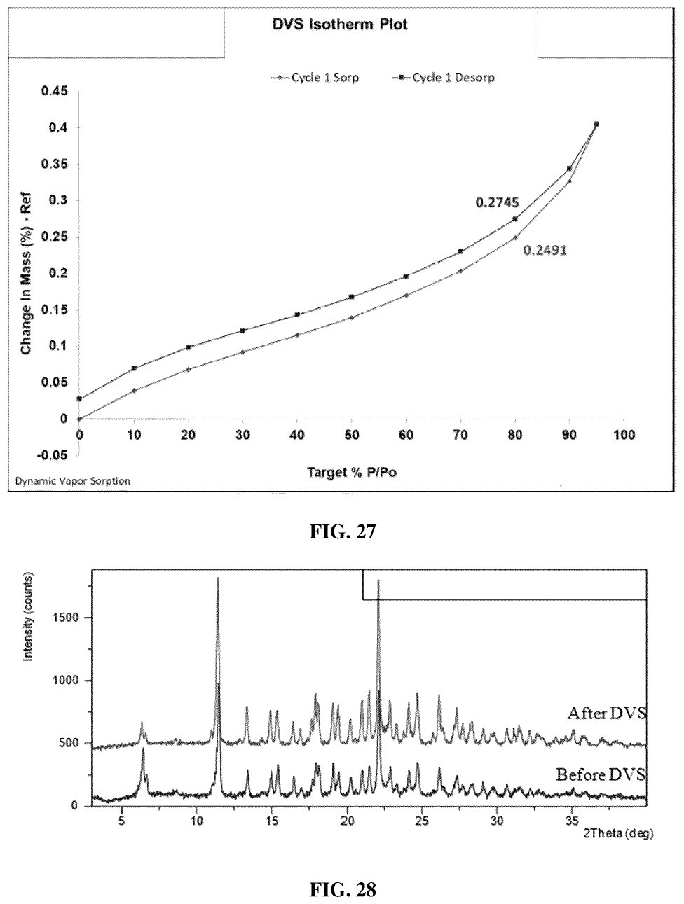

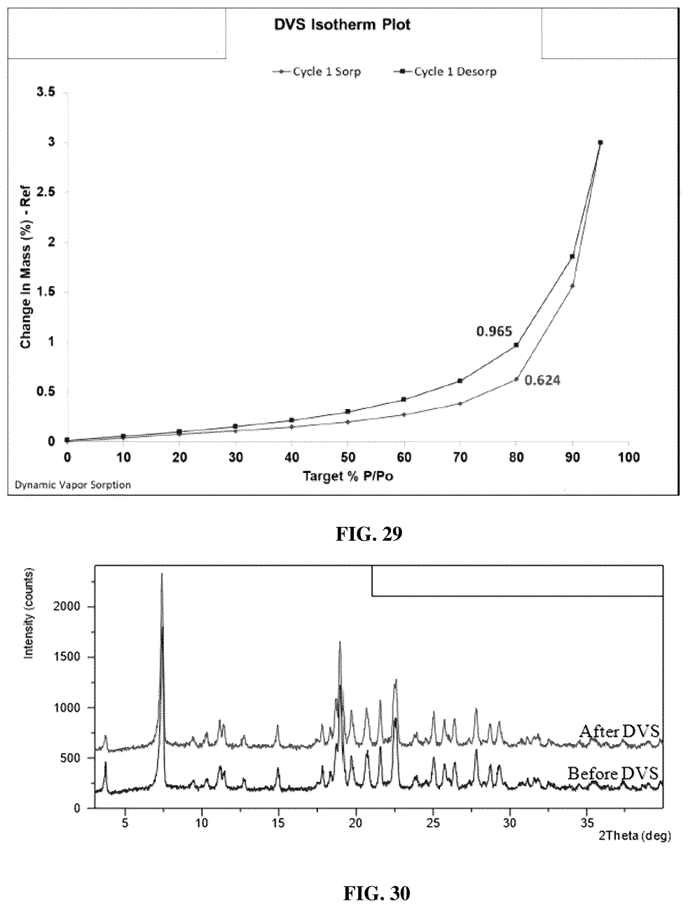

Compound A freeform Type B showed a water uptake of 0.06% at 25° C./80% RH, indicating Compound A freeform Type B was non-hygroscopic (FIG. 23). The XRPD results (FIG. 24) showed no form change after DVS. Compound A esylate Type A showed a water uptake of 0.50% at 25° C./80% RH, indicating Compound A esylate Type A was slightly hygroscopic (FIG. 25). The XRPD results (FIG. 26) showed no form change after DVS. Compound A t-butylamine salt Type A showed a water uptake of 0.25% at 25° C./80% RH, indicating Compound A t-butylamine Type A was slightly hygroscopic (FIG. 27). The XRPD results (FIG. 28) showed no form change after DVS. Compound A tris salt Type A showed a water uptake of 0.62% at 25° C./80% RH, indicating tris salt Type A was slightly hygroscopic (FIG. 29). XRPD results (FIG. 30) showed no form change after DVS.

### 5.2.2. Kinetic Solubility

Kinetic solubility evaluation was conducted in water and three bio-relevant media (SGF, FaSSIF, and FeSSIF at 37° C. to understand the solubility and disproportionation risk of all three salts, using freeform Type B as a control. Specifically, about 35 mg samples (charge concentration 10 mg/mL, calculated by freeform) were added to 3.5 mL of each buffer. The vials were agitated at 100 rpm at 37° C. At 1 h, 4 h and 24 h, 1.0 mL of each suspension was centrifuged, the solids were analyzed by XRPD, and the supernatants were filtered and tested by HPLC for solubility and by pH meter for pH values.

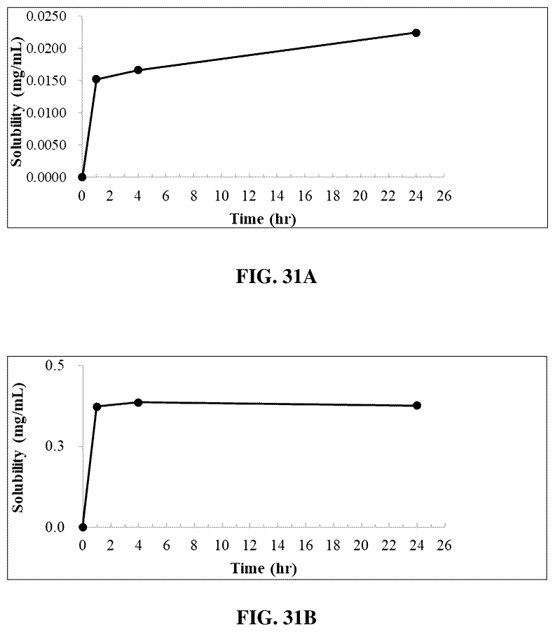

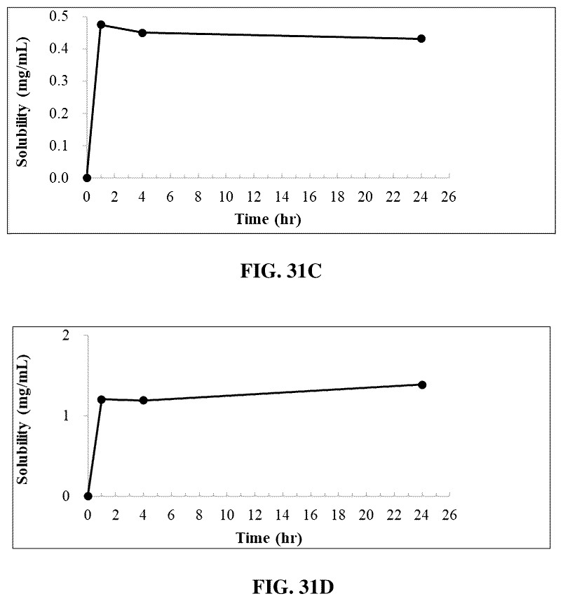

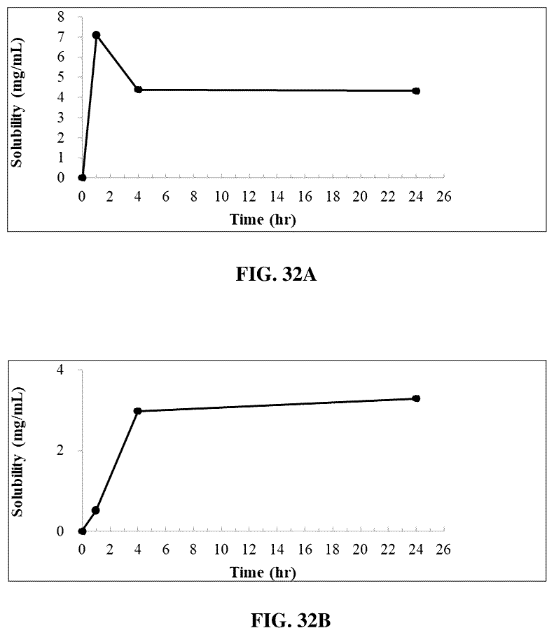

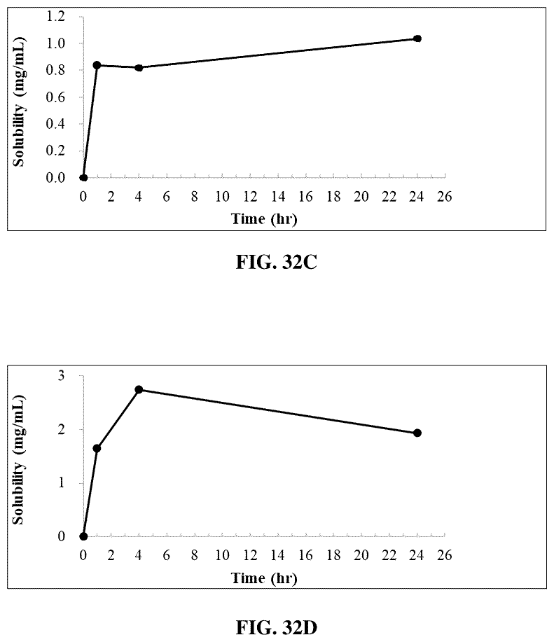

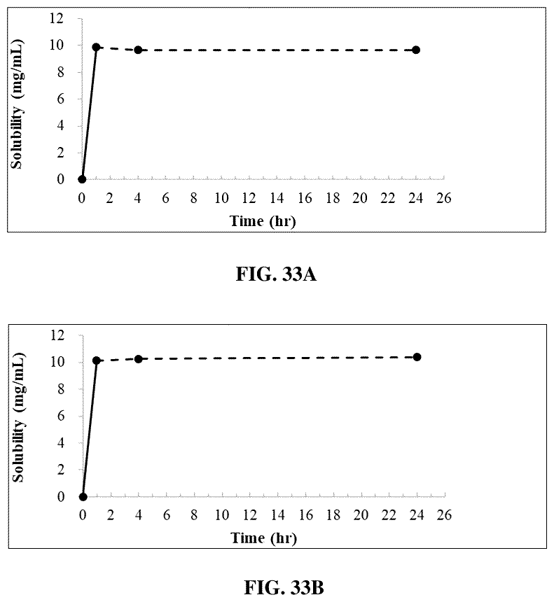

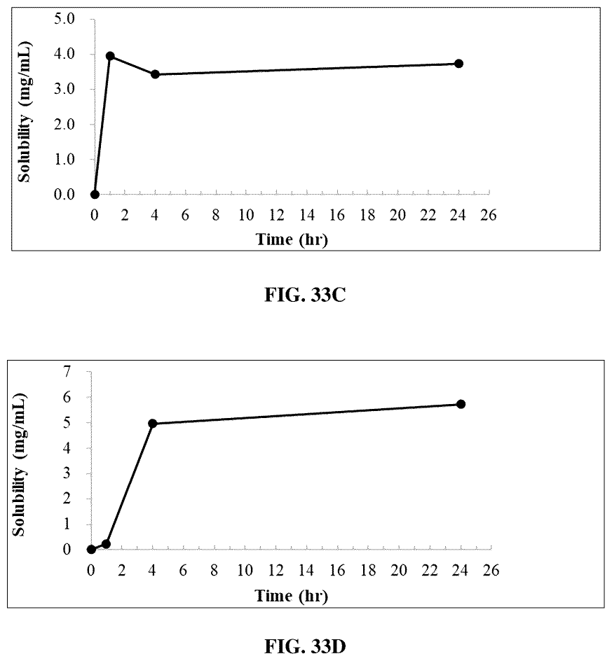

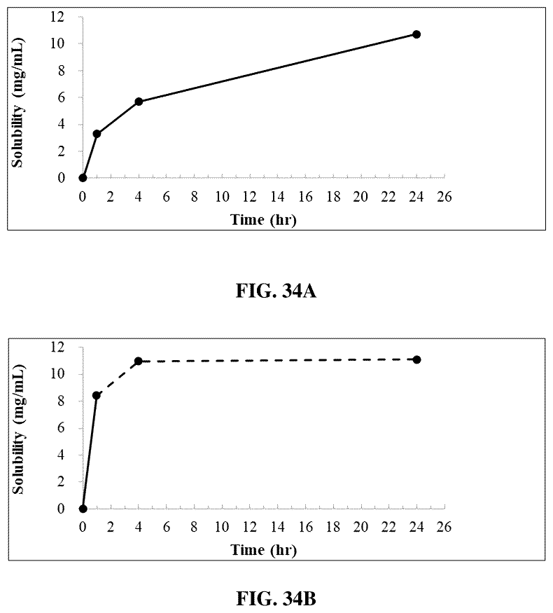

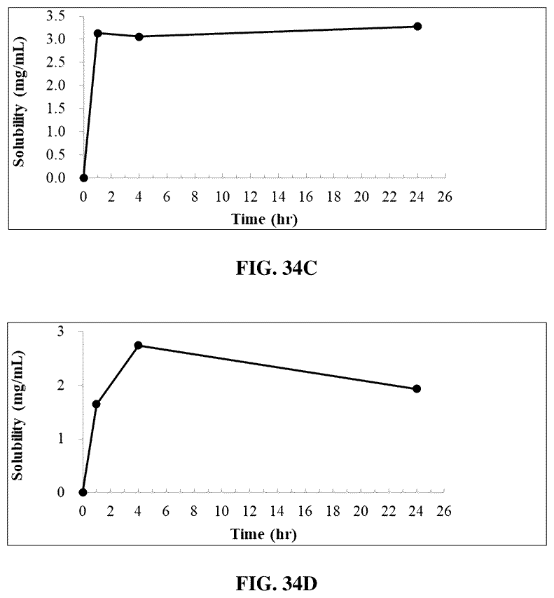

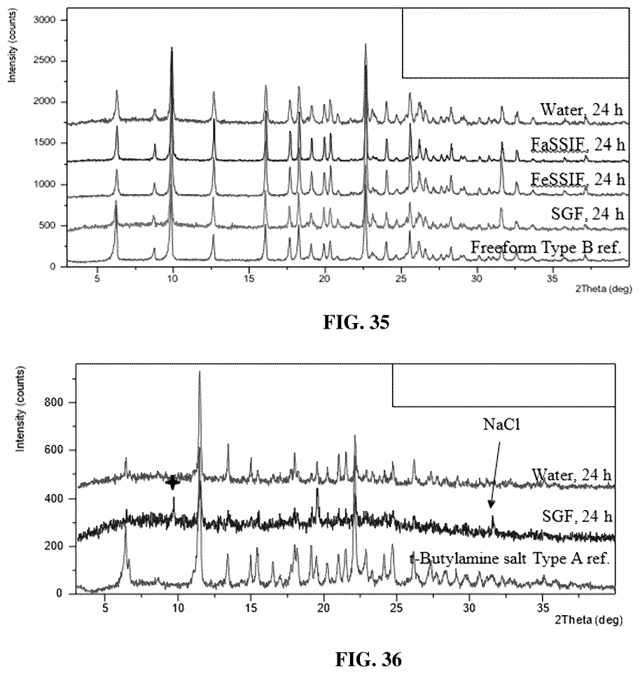

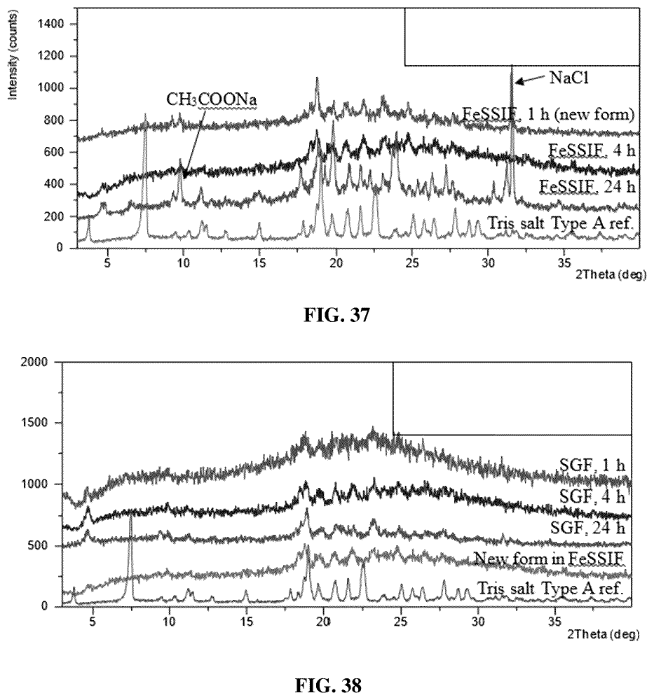

The results are summarized in Table 28 and shown in FIGS. 31A-34D. Selected XRPD results were shown in FIGS. 35-38. Compound A tris salt Type A and Compound A t-butylamine salt Type A showed higher solubility in water/FaSSIF (>10 mg/mL) and in FeSSIF (˜3 mg/mL) than Compound A esylate Type A (3-4 mg/mL in water/FaSSIF and ˜1 mg/mL in FeSSIF) and Compound A freeform Type B (0.02 mg/mL in water and ˜0.4 mg/mL in FaSSIF/FeSSIF) at 24 h. In SGF, Compound A esylate Type A showed the highest solubility (˜9 mg/mL), and Compound A tris salt Type A showed the second highest solubility (˜5 mg/mL), followed by Compound A t-butylamine salt Type A and Compound A freeform Type B (1˜2 mg/mL). No form change was observed for Compound A freeform Type B in all media up until 24 h, whereas a gel was obtained for Compound A esylate Type A in all media. Compound A tris salt Type A fully dissolved in water/FaSSIF, while a new form was observed in FeSSIF/SGF within 1 h. Compound A t-butylamine salt Type A showed no form change in water but showed an additional peak in SGF at 4 h. After 24 hr, Compound A t-butylamine salt Type A converted to gel in FeSSIF, whereas it fully dissolved in FaSSIF at 24 h.

TABLE 28
Kinetic Solubility Summary of Salts and Freeform at 37° C.

| | | 1 h | | | 4 h | | | 24 h | | |
|---|---|---|---|---|---|---|---|---|---|---|
| | | S | | Form | S | | Form | S | | Form |
| Sample | Media | (mg/mL) | pH | change | (mg/mL) | pH | change | (mg/mL) | pH | change |
| Freeform Type B | Water | 0.02 | 7.8 | No | 0.02 | 7.9 | No | 0.02 | 8.1 | No |
| Esylate Type A | | 7.10 | 1.9 | Gel | 4.39 | 1.9 | Gel | 4.33 | 1.9 | Gel |
| Tris salt Type A | | >9.85 | 6.8 | Clear | >9.64 | 6.8 | Clear | >9.63 | 6.8 | Clear |
| | | | | solution | | | solution | | | solution |
| t-Butylamine salt | | 3.29 | 6.9 | No | 5.68 | 6.8 | No | 10.73 | 6.6 | No |
| Type A | | | | | | | | | | |
| Freeform Type B | FaSSIF | 0.37 | 6.4 | No | 0.39 | 6.3 | No | 0.38 | 6.3 | No |
| Esylate Type A* | | 0.50 | 2.7 (6.3) | Gel | 2.97 | 6.2 (6.3) | Gel | 3.29 | 6.0 | Gel |
| Tris salt Type A | | >10.11 | 6.5 | Clear | >10.26 | 6.5 | Clear | >10.39 | 6.5 | Clear |
| | | | | solution | | | solution | | | solution |
| t-Butylamine salt | | 8.38 | 6.5 | Very limited | 10.96 | 6.5 | Very limited | >11.10 | 6.5 | Clear |
| Type A | | | | solids | | | solids | | | solution |
| Freeform Type B | FeSSIF | 0.47 | 4.8 | No | 0.45 | 4.8 | No | 0.43 | 4.8 | No |
| Esylate Type A | | 0.84 | 4.6 | Gel | 0.82 | 4.6 | Gel | 1.04 | 4.6 | Gel |
| Tris salt Type A | | 3.94 | 5.1 | Yes (new | 3.42 | 5.1 | Yes (new | 3.72 | 5.1 | Yes (new |
| | | | | form) | | | form) | | | form) |
| t-Butylamine salt | | 3.13 | 5.0 | Gel | 3.05 | 5.1 | Gel | 3.28 | 5.1 | Gel |
| Type A | | | | | | | | | | |
| Freeform Type B | SGF | 1.20 | 1.6 | No | 1.19 | 1.6 | No | 1.39 | 1.6 | No |
| Esylate Type A | | 9.56 | 1.5 | Gel | 9.18 | 1.5 | Gel | 9.08 | 1.5 | Gel |
| Tris salt Type A* | | 0.23 | 6.3 (1.6) | Yes (new | 4.96 | 1.7 | Yes (new | 5.72 | 1.8 | Yes (new |
| | | | | form) | | | form) | | | form) |
| t-Butylamine salt | | 1.65 | 2.2 (1.7) | No (low | 2.74 | 1.8 | Yes (low | 1.93 | 2.3 | Yes (low |
| Type A* | | | | crystallinity) | | | crystallinity + | | | crystallinity + |
| | | | | | | | peak) | | | peak) |

S: Solubility (mg/mL), calculated as freeform.
*Adjust the pH at 1 h and 4 h using 1N NaOH or 1N HCl. The pH after adjusting was shown in the parentheses. Initial pH of bio-relevant media was: SGF (1.6), FaSSIF (6.4), FeSSIF (4.9)

### 5.2.3. Physicochemical Stability

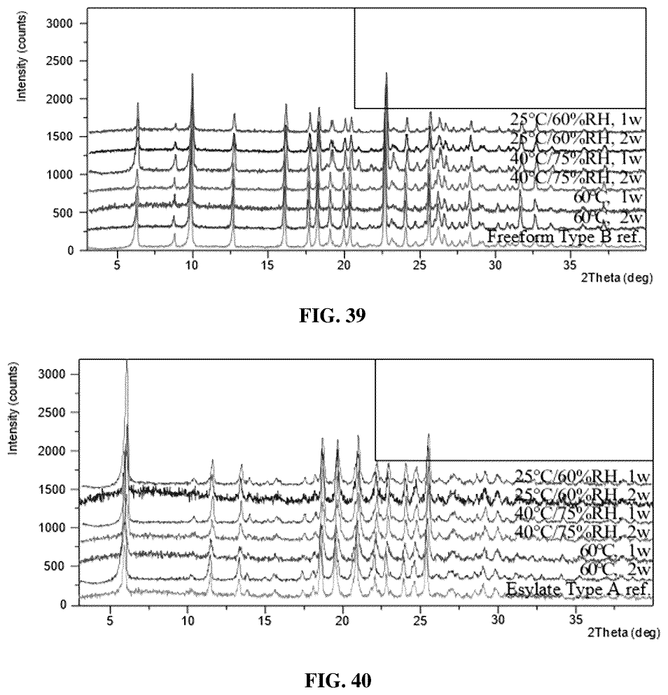

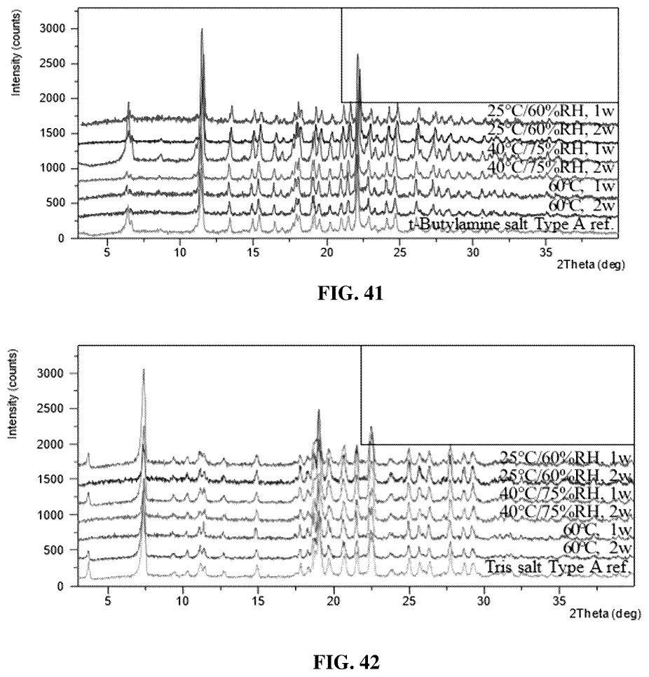

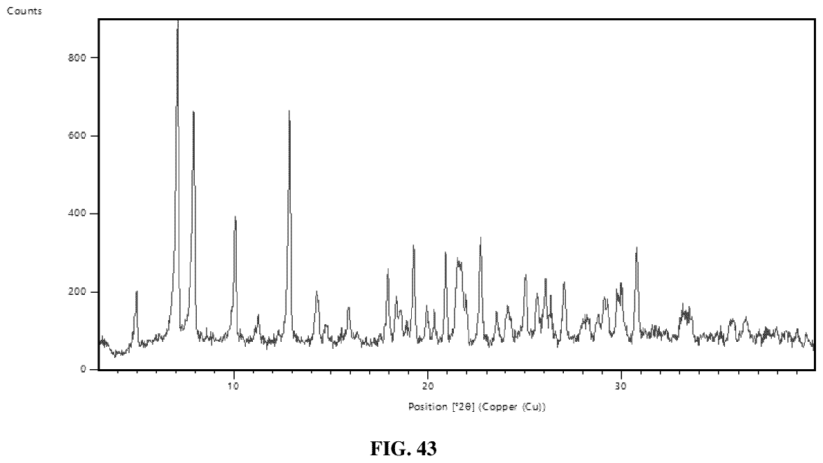

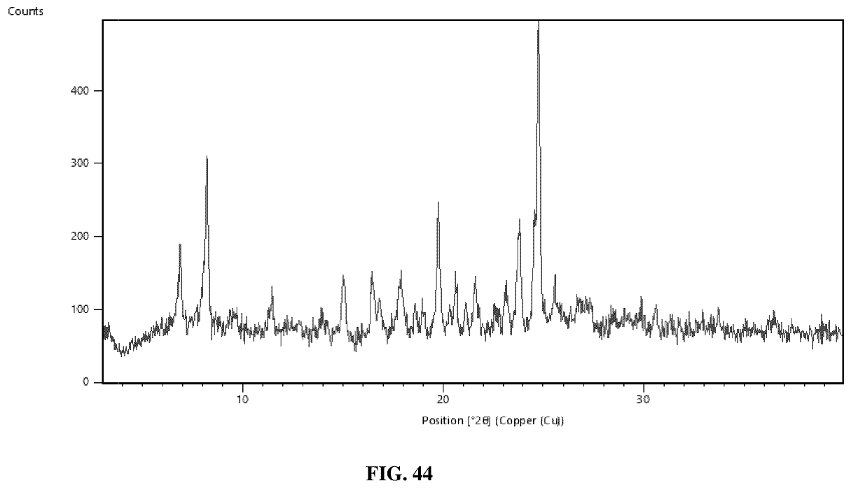

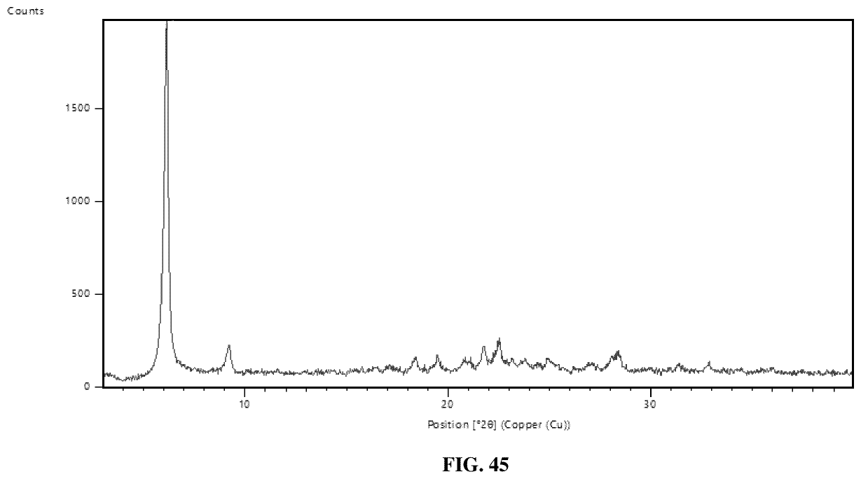

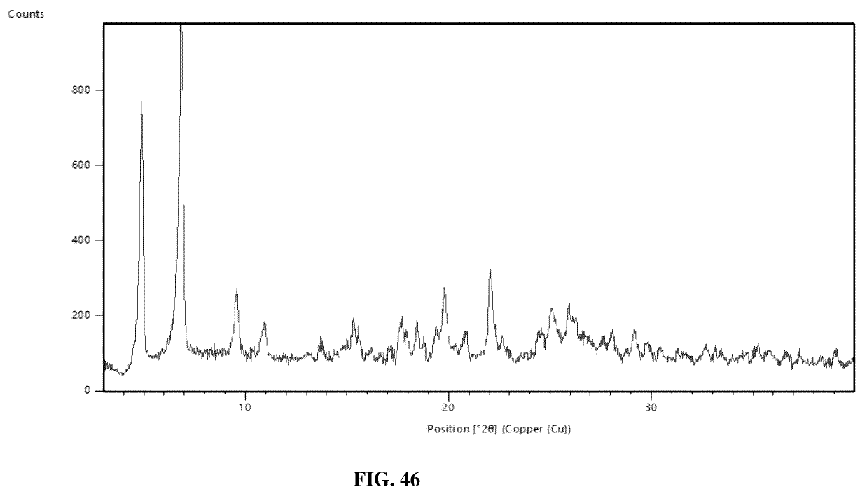

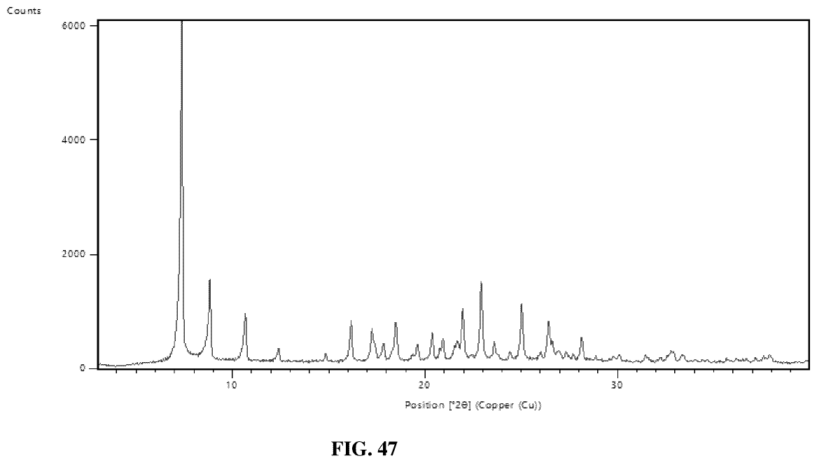

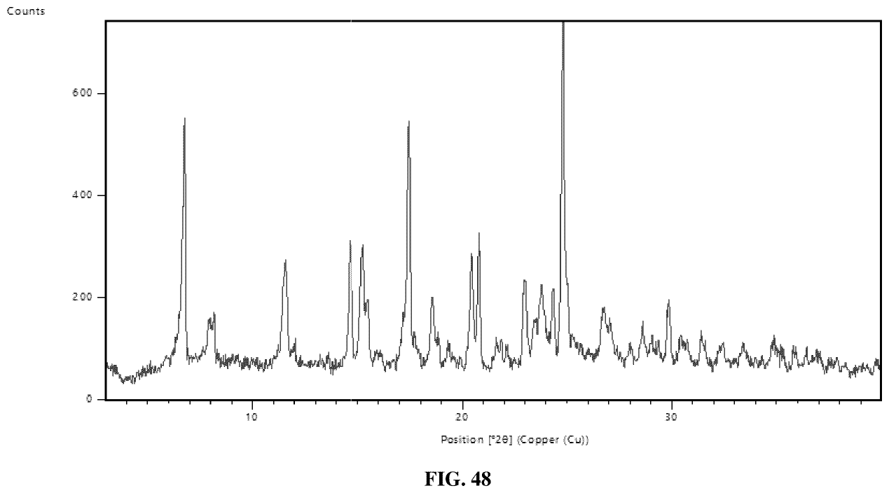

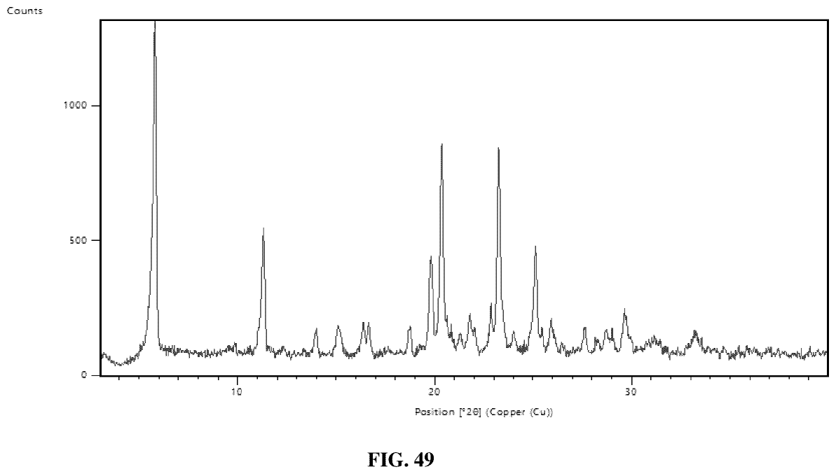

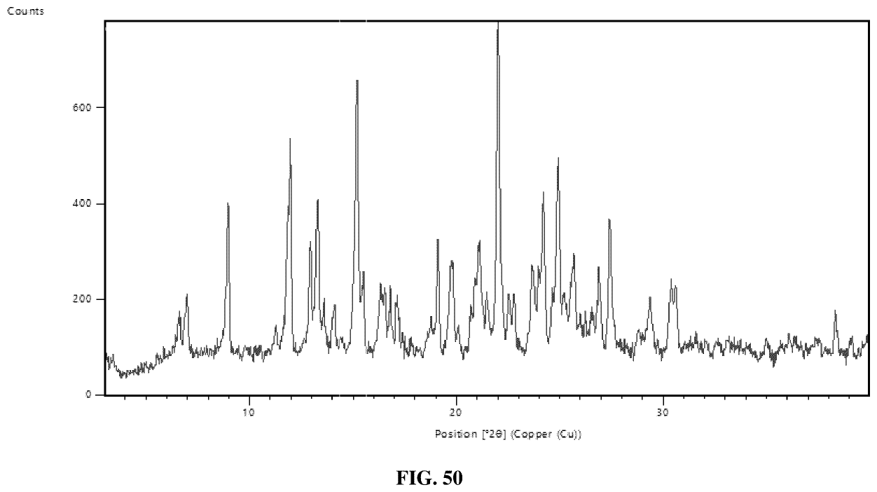

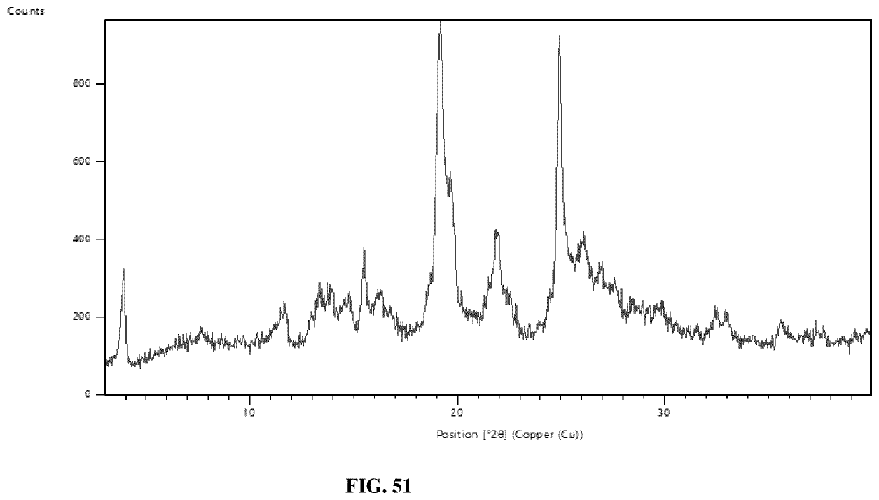

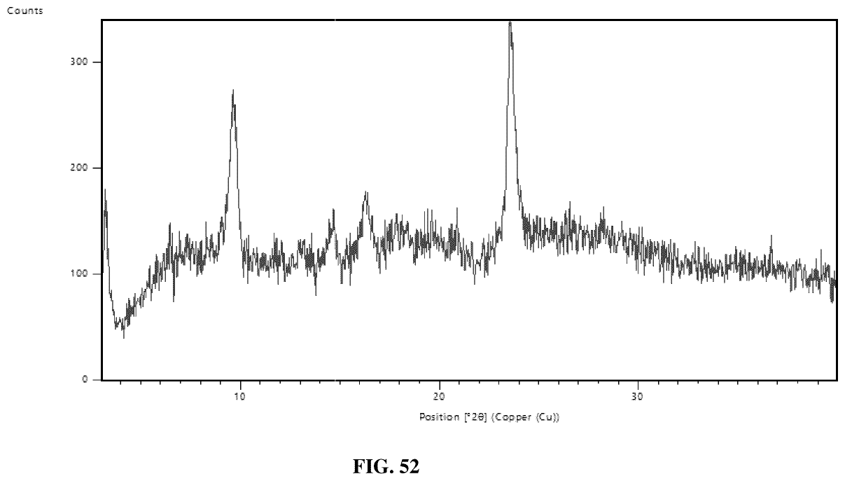

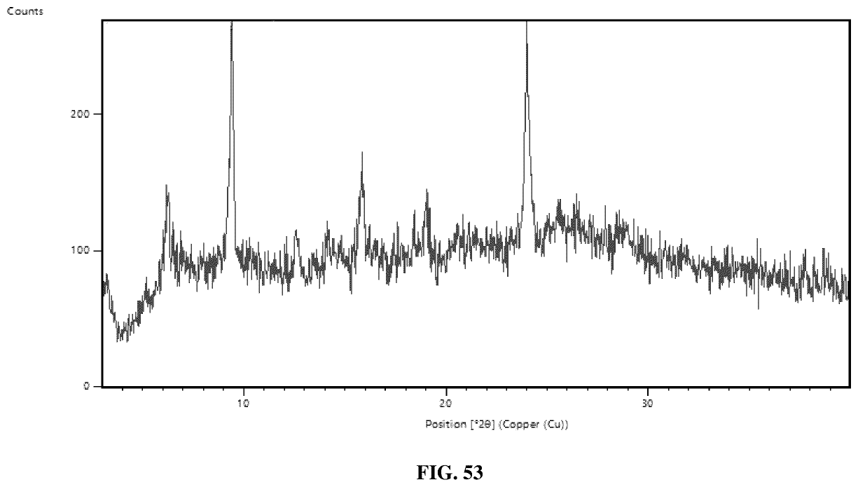

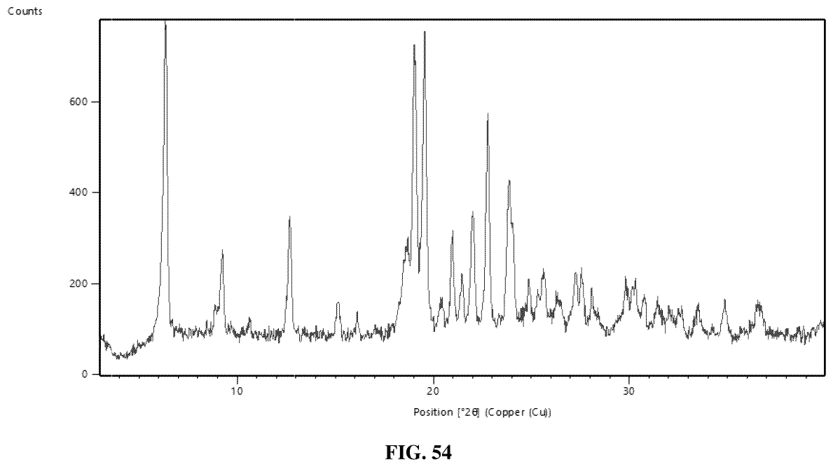

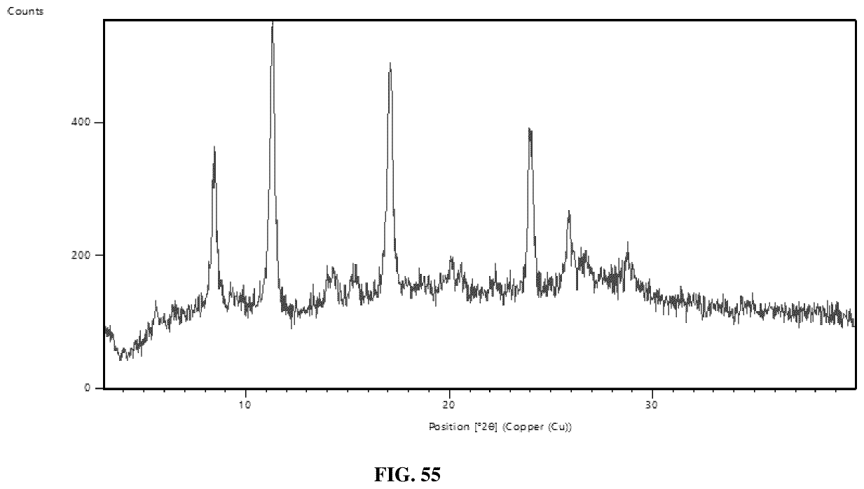

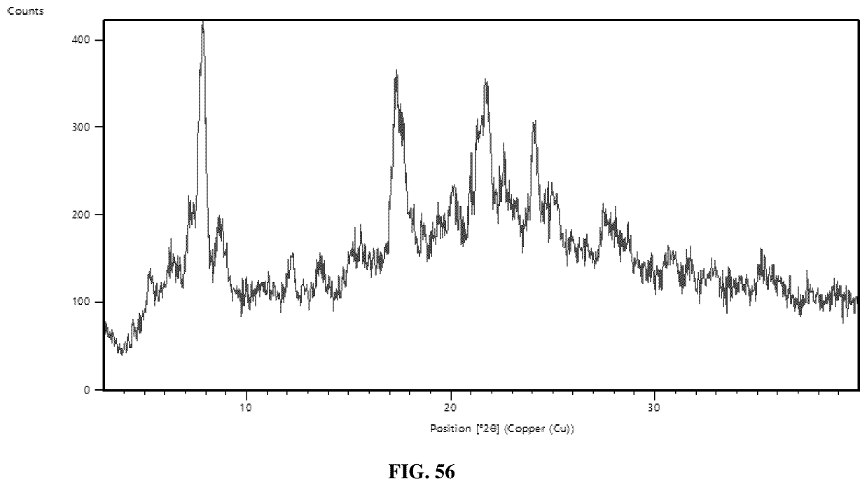

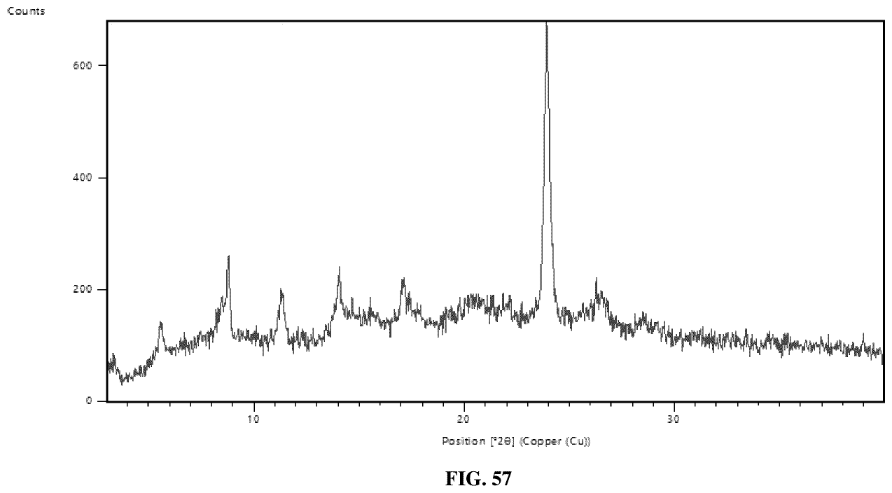

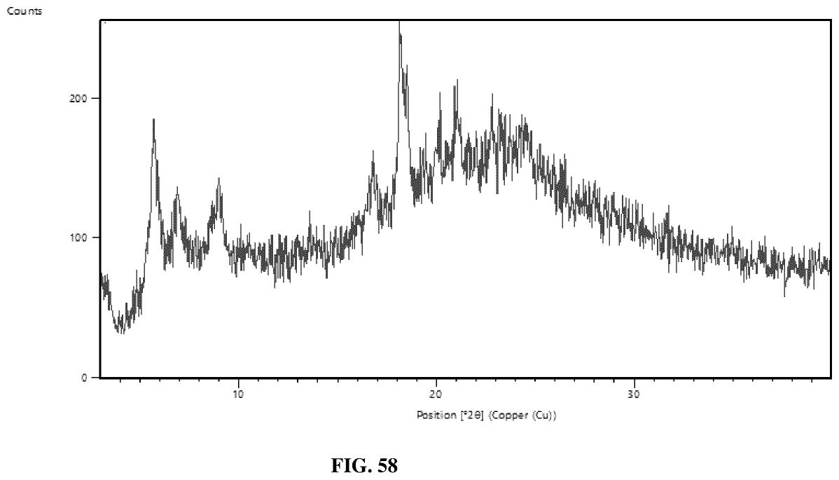

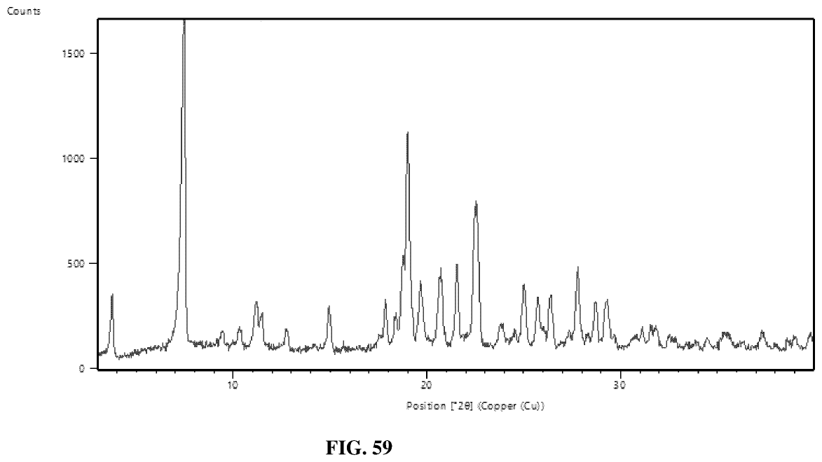

Physicochemical stability of three salt forms and freeform was evaluated under 25° C./60% RH, 40° C./75% RH and 60° C. (open condition) for two weeks. At 1-week and 2-week timepoint, samples were tested by XRPD to check the form stability and by HPLC to detect the chemical stability. The results were summarized in Table 29, and the XRPD results were displayed in FIGS. 39-42. No significant decrease in HPLC purity or form change was observed for all the salt forms and freeform Type B under all conditions for two weeks.

TABLE 29
Physicochemical Stability Results

| | Initial | | 1 week | | 2 weeks | |
|---|---|---|---|---|---|---|
| | purity | | Purity | Form | Purity | Form |
| Salt/Form | (area %) | Condition | (area %) | change | (area %) | change |
| Freeform Type | 99.06 | 25° C./60% RH | 99.06 | No | 99.04 | No |
| B | | 40° C./75% RH | 99.08 | No | 98.86 | No |
| | | 60° C. | 99.02 | No | 99.00 | No |
| Esylate Type | 97.28 | 25° C./60% RH | 97.15 | No | 97.35 | No |
| A | | 40° C./75% RH | 97.31 | No | 97.28 | No |
| | | 60° C. | 96.85 | No | 97.26 | No |
| t-Butylamine salt | 97.44 | 25° C./60% RH | 99.06 | No | 99.04 | No |
| Type A | | 40° C./75% RH | 99.08 | No | 98.86 | No |
| | | 60° C. | 99.02 | No | 99.00 | No |
| Tris salt Type A | 98.04 | 25° C./60% RH | 97.15 | No | 97.35 | No |
| | | 40° C./75% RH | 97.31 | No | 97.28 | No |
| | | 60° C. | 96.85 | No | 97.26 | No |

The results in Table 29 illustrate that no significant decrease in purity or form change was observed for all three salts and freeform Type B at all conditions (25° C./60% RH, 40° C./75% RH and 60° C. (open condition)) for up to 2 weeks.

In sum, the solid forms of Compound A as the freeform (Type B), esylate, t-butylamine salt, and tris salt demonstrated superior solid-state properties based on reproducibility, high crystallinity, negligible TGA weight loss, neat DSC signals, and high physicochemical stability.

### 6. EQUIVALENTS AND INCORPORATION BY REFERENCE

While aspects of this disclosure have been particularly shown and described with reference to a preferred embodiment and various alternate embodiments, it will be understood by persons skilled in the relevant art that various changes in form and details can be made therein without departing from the scope of the disclosure.

All references, issued patents, and patent applications cited within the body of the instant specification are hereby incorporated by reference in their entirety, for all purposes.

1. A compound, wherein the compound is a solid form of Compound A:

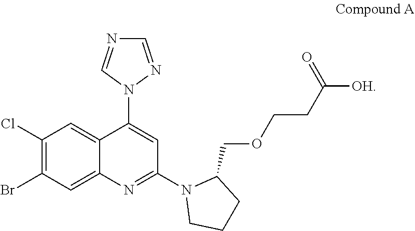 [SMILES: O=C(O)CCOC[C@@H]1CCCN1c1cc(-n2cncn2)c2cc(Cl)c(Br)cc2n1]

2-8. (canceled)

9. A compound, wherein the compound is a pharmaceutically acceptable salt of Compound A: 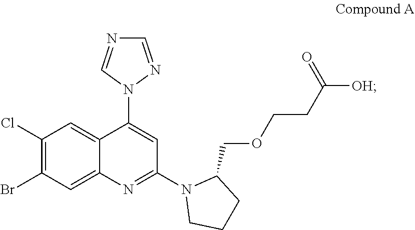 [SMILES: O=C(O)CCOC[C@@H]1CCCN1c1cc(-n2cncn2)c2cc(Cl)c(Br)cc2n1]
wherein the pharmaceutically acceptable salt is selected from a hydrochloride, sulfate, napadisylate, esylate, mesylate, napsylate, besylate, sodium, potassium, L-arginine, choline, L-lysine, t-butylamine, meglumine, and tris(hydroxymethyl)aminomethane salt.

10-14. (canceled)

15. The compound of claim 9, wherein the pharmaceutically acceptable salt is an esylate salt.

16-17. (canceled)

18. The compound of claim 15, wherein Compound A esylate is a solid form and has an X-ray diffraction pattern with at least three characteristic peaks expressed in values of degrees 2θ selected from those at 6.0±0.2, 11.5±0.2, 13.3±0.2, 18.6±0.2, 19.5±0.2, 20.9±0.2, 22.1±0.2, 22.8±0.2, 23.9±0.2, 24.6±0.2, 25.4±0.2, and 29.1±0.2.

19-21. (canceled)

22. The compound of claim 9, wherein the pharmaceutically acceptable salt is a t-butylamine salt.

23-24. (canceled)

25. The compound of claim 22, wherein Compound A t-butylamine salt is a solid form and has an X-ray diffraction pattern with at least three characteristic peaks expressed in values of degrees 2θ selected from those at 6.4±0.2, 6.6±0.2, 11.4±0.2, 13.4±0.2, 15.0±0.2, 17.7±0.2, 17.9±0.2, 21.0±0.2, 21.4±0.2, 22.1±0.2, and 26.1±0.2.

26-28. (canceled)

29. The compound of claim 9, wherein the pharmaceutically acceptable salt is tris(hydroxymethyl)aminomethane salt.

30-31. (canceled)

32. The compound of claim 29, wherein Compound A tris(hydroxymethyl)aminomethane salt is a solid form and has an X-ray diffraction pattern with at least three characteristic peaks expressed in values of degrees 2θ selected from those at 3.7±0.2, 7.5±0.2, 11.2±0.2, 15.0±0.2, 17.8±0.2, 18.4±0.2, 18.8±0.2, 19.0±0.2, 19.7±0.2, 20.6±0.2, 21.5±0.2, 22.4±0.2, 22.6±0.2, 25.0±0.2, 25.7±0.2, 26.4±0.2, 27.8±0.2, 28.7±0.2, 29.2±0.2, and 29.3±0.2.

33-35. (canceled)

36. The compound of claim 9, wherein the pharmaceutically acceptable salt is hydrochloride salt.

37. The compound of claim 36, wherein Compound A hydrochloride salt is a solid form and is Compound A hydrochloride salt Type A and has an X-ray diffraction pattern with at least three characteristic peaks expressed in values of degrees 2θ selected from those at 7.1±0.2; 7.9±0.2, 10.0±0.2, 12.9±0.2, 17.9±0.2, 19.3±0.2, 20.9±0.2, 21.5±0.2, 22.7±0.2, 25.1±0.2, 26.0±0.2, 27.0±0.2, and 30.7±0.2.

38. The compound of claim 36, wherein Compound A hydrochloride salt is a solid form and is Compound A hydrochloride salt Type B and has an X-ray diffraction pattern with at least three characteristic peaks expressed in values of degrees 2θ selected from those at 6.9±0.2, 8.2±0.2, 15.0±0.2, 19.8±0.2, 23.8±0.2, and 24.7±0.2.

39. The compound of claim 36, wherein Compound A hydrochloride salt is a solid form and is Compound A hydrochloride salt Type C and has an X-ray diffraction pattern with at least three characteristic peaks expressed in values of degrees 2θ selected from those at 6.1±0.2, 9.2±0.2, 18.4±0.2, 19.5±0.2, 21.8±0.2, 22.5±0.2, 23.8±0.2, and 25.0±0.2, 28.4±0.2.

40. The compound of claim 36, wherein Compound A hydrochloride salt is a solid form and is Compound A hydrochloride salt Type D and has an X-ray diffraction pattern with at least three characteristic peaks expressed in values of degrees 2θ selected from those at 4.9±0.2, 6.9±0.2, 9.5±0.2, 19.8±0.2, 22.0±0.2, 25.1±0.2, and 25.9±0.2.

41. (canceled)

42. The compound of claim 9, wherein the pharmaceutically acceptable salt is sulfate.

43. The compound of claim 42, wherein Compound A sulfate is a solid form and has an X-ray diffraction pattern with at least three characteristic peaks expressed in values of degrees 2θ selected from those at 3.4±0.2, 5.7±0.2, 6.9±0.2, 9.0±0.2, 16.8±0.2, 18.2±0.2, 20.1±0.2, 21.0±0.2, and 24.6±0.2.

44. The compound of claim 9, wherein the pharmaceutically acceptable salt is napadisylate.

45. The compound of claim 44, wherein Compound A napadisylate is a solid form and has an X-ray diffraction pattern with at least three characteristic peaks expressed in values of degrees 2θ selected from those at 6.7±0.2, 8.0±0.2, 11.6±0.2, 14.7±0.2, 15.3±0.2, 15.5±0.2, 17.5±0.2, 18.6±0.2, 20.4±0.2, 20.8±0.2, 23.0±0.2, 23.4±0.2, 23.8±0.2, 24.3±0.2, 24.8±0.2, 26.7±0.2, 28.6±0.2, and 29.9±0.2.

46. The compound of claim 9, wherein the pharmaceutically acceptable salt is mesylate.

47. The compound of claim 46, wherein Compound A mesylate is a solid form and has an X-ray diffraction pattern with at least three characteristic peaks expressed in values of degrees 2θ selected from those at 5.8±0.2, 11.3±0.2, 16.4±0.2, 19.8±0.2, 20.4±0.2, 21.8±0.2, 22.8±0.2, 23.3±0.2, 25.1±0.2, 25.9±0.2, and 29.7±0.2.

48. The compound of claim 9, wherein the pharmaceutically acceptable salt is napsylate.

49. The compound of claim 48, wherein Compound A napsylate is a solid form and has an X-ray diffraction pattern with at least three characteristic peaks expressed in values of degrees 2θ selected from those at 9.0±0.2, 12.0±0.2, 13.0±0.2, 13.3±0.2, 15.2±0.2, 16.3±0.2, 16.8±0.2, 19.1±0.2, 19.8±0.2, 21.1±0.2, 22.0±0.2, 22.8±0.2, 23.7±0.2, 24.0±0.2, 24.2±0.2, 24.9±0.2, 25.7±0.2, 26.9±0.2, 27.4±0.2, 29.4±0.2, and 30.4±0.2.

50. The compound of claim 9, wherein the pharmaceutically acceptable salt is besylate.

51. The compound of claim 50, wherein Compound A besylate is a solid form and has an X-ray diffraction pattern with at least three characteristic peaks expressed in values of degrees 2θ selected from those at 3.9±0.2, 13.3±0.2, 13.9±0.2, 14.8±0.2, 15.5±0.2, 16.3±0.2, 19.1±0.2, 19.7±0.2, 21.9±0.2, 24.9±0.2, 26.0±0.2, 26.9±0.2, and 27.6±0.2.

52. The compound of claim 9, wherein the pharmaceutically acceptable salt is sodium salt.

53. The compound of claim 52, wherein Compound A sodium salt is a solid form and has an X-ray diffraction pattern with at least three characteristic peaks expressed in values of degrees 2θ selected from those at 3.2±0.2, 9.7±0.2, 13.2±0.2, 14.6±0.2, 16.2±0.2, 18.1±0.2, and 23.6±0.2.

54. The compound of claim 9, wherein the pharmaceutically acceptable salt is potassium salt, form.

55. The compound of claim 54, wherein Compound A potassium salt is a solid form and has an X-ray diffraction pattern with at least three characteristic peaks expressed in values of degrees 2θ selected from those at 6.2±0.2, 9.4±0.2, 12.6±0.2, 15.9±0.2, 19.0±0.2, 24.0±0.2, and 37.7±0.2.

56. The compound of claim 9, wherein the pharmaceutically acceptable salt is L-arginine salt.

57. The compound of claim 56, wherein Compound A L-arginine salt is a solid form and has an X-ray diffraction pattern with at least three characteristic peaks expressed in values of degrees 2θ selected from those at 6.3±0.2, 9.3±0.2, 12.7±0.2, 18.6±0.2, 19.1±0.2, 19.6±0.2, 21.0±0.2, 21.5±0.2, 22.0±0.2, 22.8±0.2, 23.9±0.2, 24.1±0.2, 24.9±0.2, 25.7±0.2, 27.3±0.2, 27.6±0.2, 28.1±0.2, 29.8±0.2, and 30.3±0.2.

58. The compound of claim 9, wherein the pharmaceutically acceptable salt is choline salt.

59. The compound of claim 58, wherein Compound A choline salt is a solid form and has an X-ray diffraction pattern with at least three characteristic peaks expressed in values of degrees 2θ selected from those at 5.6±0.2, 8.4±0.2, 11.4±0.2, 14.4±0.2, 15.3±0.2, 17.1±0.2, 20.3±0.2, 24.0±0.2, 25.9±0.2, and 28.8±0.2.

60. The compound of claim 9, wherein the pharmaceutically acceptable salt is L-lysine salt.

61. The compound of claim 60, wherein Compound A L-lysine salt is a solid form and has an X-ray diffraction pattern with at least three characteristic peaks expressed in values of degrees 2θ selected from those at 5.2±0.2, 7.9±0.2, 8.7±0.2, 12.2±0.2, 13.6±0.2, 15.4±0.2, 17.3±0.2, 20.1±0.2, 21.8±0.2, 22.6±0.2, 24.0±0.2, 25.1±0.2, and 27.5±0.2.

62. The compound of claim 9, wherein the pharmaceutically acceptable salt is meglumine salt.

63. The compound of claim 62, wherein Compound A meglumine salt is a solid form and has an X-ray diffraction pattern with at least three characteristic peaks expressed in values of degrees 2θ selected from those at 3.4±0.2, 5.6±0.2, 8.8±0.2, 11.3±0.2, 14.0±0.2, 17.1±0.2, 20.5±0.2, 22.0±0.2, 24.0±0.2, 26.5±0.2, and 28.8±0.2.

64-65. (canceled)

66. A method of antagonizing cyclic GMP-AMP synthase (cGAS) in a patient in need thereof, comprising administering an effective amount of a compound of claim 9.

67. A method of treating an inflammatory, allergic, autoimmune, or neurodegenerative disease in a patient in need thereof, comprising administering an effective amount of a compound of claim 9.

68-70. (canceled)
